SEQUENCE LISTING (16 sequences)

SEQ ID NO: 1 - DNA, 23 bp, Artificial sequence
  Synthetic
ggagcgcaccatcttcttcaagg

SEQ ID NO: 2 - protein, 1368 aa, Streptococcus pyogenes [sequence omitted]

SEQ ID NO: 3 - DNA, 22 bp, Artificial sequence
  Synthetic
gtatagcatacattatacgagg

SEQ ID NO: 4 - DNA, 23 bp, Artificial sequence
  Synthetic
gaagcgtgatgacaaagaggagg

SEQ ID NO: 5 - DNA, 124 bp, Artificial sequence
  Synthetic
gtttttaatacgactcactataggagcgcaccatcttcttcagttttagagctagaaatagcaagttaaaataaggctagtccgttatcaacttgaaaaagtggcaccgagtcggtgctttttt

SEQ ID NO: 6 - DNA, 124 bp, Artificial sequence
  Synthetic
gtttttaatacgactcactataggtatagcatacattatacggttttagagctagaaatagcaagttaaaataaggctagtccgttatcaacttgaaaaagtggcaccgagtcggtgctttttt

SEQ ID NO: 7 - DNA, 124 bp, Artificial sequence
  Synthetic
gtttttaatacgactcactatagaagcgtgatgacaaagagggttttagagctagaaatagcaagttaaaataaggctagtccgttatcaacttgaaaaagtggcaccgagtcggtgctttttt

SEQ ID NO: 8 - DNA, 22 bp, Artificial sequence
  Synthetic
ctggtcgagctggacggcgacg

SEQ ID NO: 9 - DNA, 22 bp, Artificial sequence
  Synthetic
cacgaactccagcaggaccatg

SEQ ID NO: 10 - DNA, 32 bp, Artificial sequence
  Synthetic
ggacggatgacaataccaggcaggataaggcc

SEQ ID NO: 11 - DNA, 180 bp, Artificial sequence
  Synthetic
cctggtcatgggttaccagaagaaactgagaagcatgacggacaagtacaggctgcacctgtcagtggccgacctcctaagcttggatccctttgtcatcacgcttcccttctgggcagttgatgccgtggcaaactggtactttgggaacttcctatgcaaggcagtccatgtcatcta

SEQ ID NO: 12 - DNA, 29 bp, Artificial sequence
  Synthetic
ccgacctcctctttgtcatcacgcttccc

SEQ ID NO: 13 - DNA, 27 bp, Artificial sequence
  Synthetic
ccgacctcctttgtcatcacgcttccc

SEQ ID NO: 14 - DNA, 29 bp, Artificial sequence
  Synthetic
  misc_feature (11)..(11): n is a, c, g, or t
ccgacctcctntttgtcatcacgcttccc

SEQ ID NO: 15 - DNA, 28 bp, Artificial sequence
  Synthetic
ccgacctccttttgtcatcacgcttccc

SEQ ID NO: 16 - DNA, 29 bp, Artificial sequence
  Synthetic
  misc_feature (11)..(22): n is a, c, g, or t
ccgacctcctnnnnnnnnnnnnctttgtc

(19) United States
(12) Patent Application Publication
(10) Pub. No.: US 2022/0395589 A1
(43) Pub. Date: Dec. 15, 2022

(54) POLYMERIC NANOPARTICLES FOR INTRACELLULAR PROTEIN DELIVERY

(71) Applicant: The Johns Hopkins University, Baltimore, MD (US)

(72) Inventors: Jordan J. Green, Baltimore, MD (US); David Wilson, Baltimore, MD (US); Yuan Rui, Baltimore, MD (US)

(21) Appl. No.: 17/771,142

(22) PCT Filed: Oct. 26, 2020
(86) PCT No.: PCT/US2020/057354

§ 371 Date: Apr. 22, 2022

Related U.S. Application Data
(60) Provisional application No. 62/925,855, filed on Oct. 25, 2019.

(51) Int. Cl. A61K 49/00; A61K 9/16; A61K 38/47; A61K 38/16; A61K 38/46; A61K 31/7088

(52) U.S. Cl. CPC A61K 49/0093; A61K 9/1647; A61K 38/47; A61K 38/164; A61K 38/168; A61K 38/465; A61K 31/7088

(57) ABSTRACT

Cationic polymers having one or more anionic ligand end groups, including a new class of carboxylated branched poly(beta-amino ester)s that can self-assemble into nanoparticles for efficient intracellular delivery of different biomolecules, including a variety of proteins is disclosed.

### FEDERALLY SPONSORED RESEARCH OR DEVELOPMENT

This invention was made with government support under CA228133 and EB022148 awarded by the National Institutes of Health. The government has certain rights in the invention.

### BACKGROUND

Direct intracellular delivery of proteins, including therapeutic proteins, to target cells is safer than plasmid delivery as it eliminates risks of insertional mutagenesis. In the context of gene editing, there is the added benefit that delivery of CRISPR/Cas9 ribonucleoproteins (RNPs) to target cells reduces the probability of off-target editing by reducing RNP persistence time. Intracellular protein delivery faces many challenges, however, as the large size and hydrophilicity of such proteins make them generally membrane impermeable. Poly(beta-amino ester)s (PBAEs) are biodegradable, cationic polymers that self-assemble into nanoparticles with nucleic acids via electrostatic interactions and have been developed for effective nucleic acid delivery. In contrast to nucleic acids, however, different proteins carry different surface charges and nanoparticle encapsulation of such proteins cannot rely solely on charge interactions.

### SUMMARY

The presently disclosed subject matter provides cationic polymers having one or more anionic ligand end groups and their use for delivering one or more biomolecules to a cell.

In some aspects, the composition comprises a cationic polymer having one or more anionic end groups. In certain aspects, the cationic polymer comprises a naturally-derived cationic polymer. In other aspects, the cationic polymer comprises a synthetic cationic polymer.

In particular aspects, the one or more anionic end groups is selected from the group consisting of an amide-linked carboxylate, an ester-linked carboxylate, an ester-ethylene glycol-linked carboxylate, an amide-ethylene glycol-linked carboxylate, an ester-linked phosphate, an ester-ethylene glycol-linked phosphate, an amide-ethylene glycol-linked phosphate, an ester-linked sulfonate, and an ester-ethylene glycol-linked sulfonate, an amide-ethylene glycol-linked sulfonate.

In yet more particular aspects, the presently disclosed subject matter provides a branched poly(beta-amino ester) (PBAE) of formula (I) or formula (II):

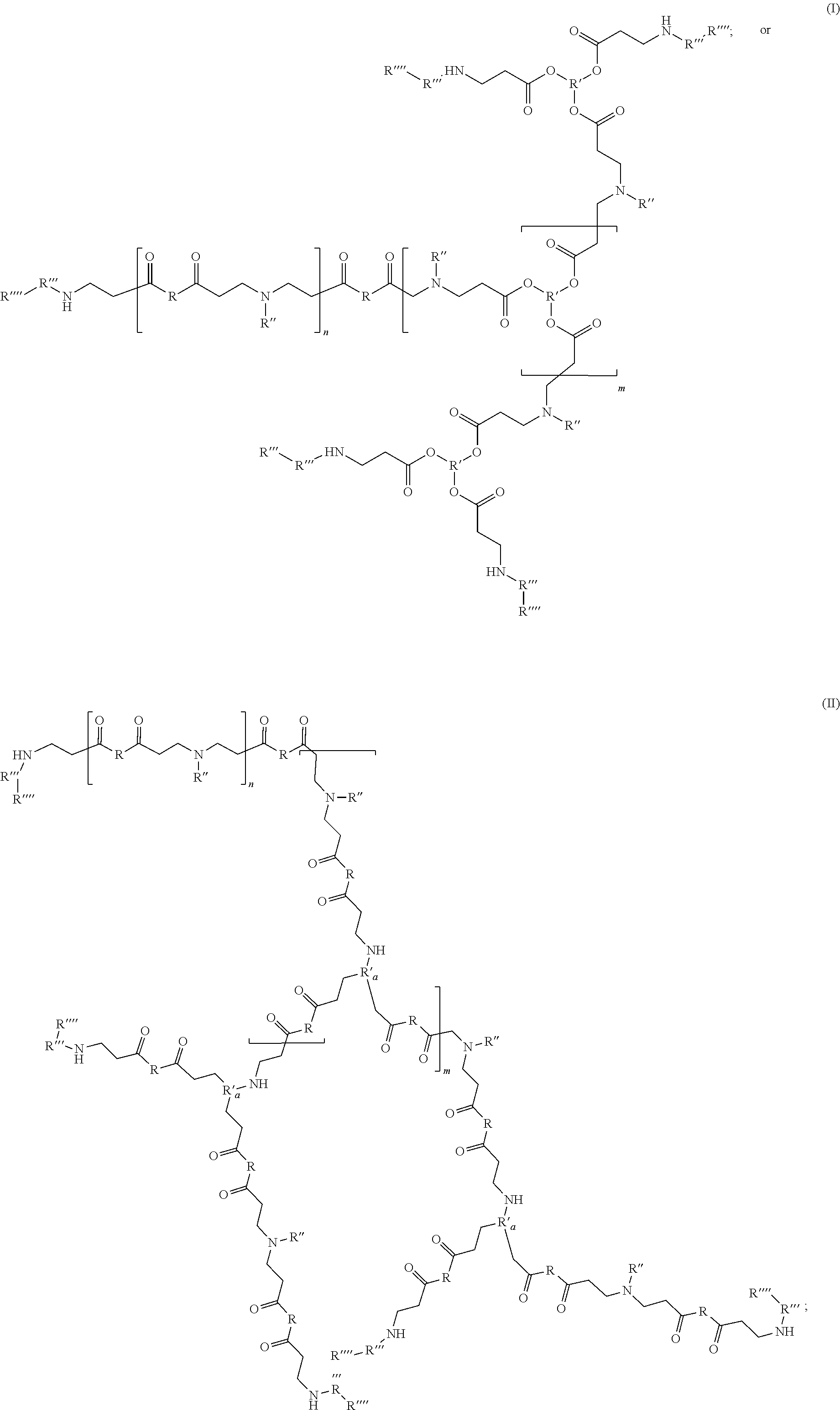

wherein: n and m are each independently an integer from 1 to 10,000; each R is independently a diacrylate monomer of the following structure:

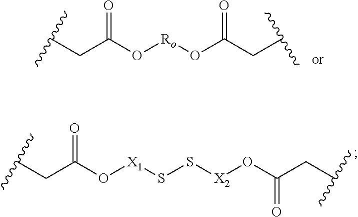

wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain; each R′ of a compound of formula (I) is a trivalent group of a triacrylate monomer having the following structure:

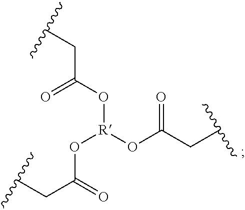

each R′ₐ of a compound of formula (II) is a tri-functional amine; each R″ is independently a side chain monomer comprising a primary, secondary, or tertiary amine; each R′″ is independently an end group monomer comprising a primary, secondary, or tertiary amine; each R″″ is independently an anionic end group; and pharmaceutically acceptable salts thereof.

In some aspects, the one or more biomolecules is selected from a peptide, a protein, a nucleic acid, a morpholino, and other charged or zwitterionic biomolecules or combinations thereof. In particular aspects, the one or more biomolecules is selected from the group consisting of a non-peptide based biological small molecule or a biomacromolecule selected from a sugar, a polysaccharide, a carbohydrate, a morpholino oligomer, and/or a nucleic acid.

In certain aspects, the protein is selected from the group consisting of a ribosome inactivating protein (RIP), a gene-editing protein, an immunoglobulin, a nanobody, and an intrabody. In yet more certain aspects, the gene editing protein comprises a Cas9 ribonucleoprotein (RNP).

In other aspects, the presently disclosed subject matter provides a method for delivering a protein to a cell, the method contacting a cell with the presently disclosed composition, wherein the composition comprises at least one protein.

In yet other aspects, the presently disclosed subject matter provides a method for editing a gene, comprising contacting a cell with the presently disclosed composition, wherein the composition comprises at least one gene-editing protein.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show the design and characterization of self-assembled carboxylated branched PBAE protein nanoparticles. (FIG. 1A) Assembly of carboxylated branched PBAEs with proteins. (FIG. 1B) Structures of carboxylate ligands C1, C3, C5, C7, and C10, arranged in order of increasing hydrophobicity. (FIG. 1C) Hydrodynamic diameter and zeta potentials of nanoparticles formulated with BSA (30 w/w) as measured by DLS. Data presented as mean+SEM (n=3). (FIG. 1D) Representative TEM images of C₅/BSA nanoparticles;

FIG. 2A, FIG. 2B, and FIG. 2C demonstrate that carboxylated PBAE nanoparticles mediate cytosolic protein delivery. (FIG. 2A) Average fluorescence intensity of cells treated with C5/FITC-BSA nanoparticles (300 ng FITC-BSA per well, 20 w/w). Data presented as mean+SEM (n=4). (FIG. 2B) Uptake by HEK cells in the presence of different endocytosis inhibitors; CPZ=chlorpromazine, MCD=methyl-β-cyclodextrin, GEN=genistein, CYD=cytochalasin-D. Data presented as mean±SEM; statistical significance determined by one-way ANOVA with Tukey post-hoc tests as compared to the control group (n=4). *P<0.05, **P<0.01, ****P<0.0001. (FIG. 2C) Confocal images of HEK cells treated with C5/FITC-BSA nanoparticles or protein alone for 4 h. Scale bar=10 μm;

FIG. 3A, FIG. 3B, FIG. 3C, and FIG. 3D show a Gal8-GFP recruitment assay to assess nanoparticle-mediated endosomal disruption. (FIG. 3A) Gal8 recruitment overview; in cells with intact endosomes, Gal8-GFP is dispersed throughout endosomes with no interactions with intra-endosomal glycans. Gal8-GFP binds glycans in disrupted endosomes, resulting in punctate fluorescent dots. (FIG. 3B) Gal8—GFP recruitment were quantified by image-based analysis. Individual cells were identified through nuclear staining (left); Gal8-GFP recruitment could be visualized in the green fluorescence channel (center); punctate GFP+ spots were identified and counted (red dots). (FIG. 3C) Representative images of Gal8-GFP+B16 cells treated with carboxylated PBAE/BSA nanoparticles (125 ng BSA per well, 25 w/w; scale bar=50 μm). (FIG. 3D) Endosomal disruption level quantified by the number of Gal8-GFP spots per cell. Data presented as mean±SEM; statistical significance determined by one-way ANOVA with Tukey post-hoc tests as compared to the C5 group (n=4). *P<0.05, **P<0.01, ****P<0.0001;

FIG. 4A, FIG. 4B, FIG. 4C, FIG. 4D, and FIG. 4E show carboxylated C5 polymeric nanoparticles for cytosolic delivery of different protein types. Confocal images of HEK cells treated with C5 nanoparticles encapsulating FITC-IgG (FIG. 4A) and GFP (FIG. 4B) for 4 h; 450 ng protein delivered per well at 30 w/w (scale bar=50 μm). (FIG. 4C) Functional delivery of ribosome-inactivating protein saporin resulted in significant levels of cell death; the final polymer concentration per well was 0.075 μg/μL. Data presented as mean±SEM (n=4). (FIG. 4D) Representative images of CT-2A cells treated with 10 nM naked saporin or C5/saporin nanoparticles. (FIG. 4E) Molecular weight and isoelectric point of proteins delivered by C5 nanoparticles;

FIG. 5A, FIG. 5B, FIG. 5C, FIG. 5D, FIG. 5E, FIG. 5F, and FIG. 5G demonstrate C5 nanoparticle delivery of Cas9 RNPs enable robust CRISPR gene editing in vitro.

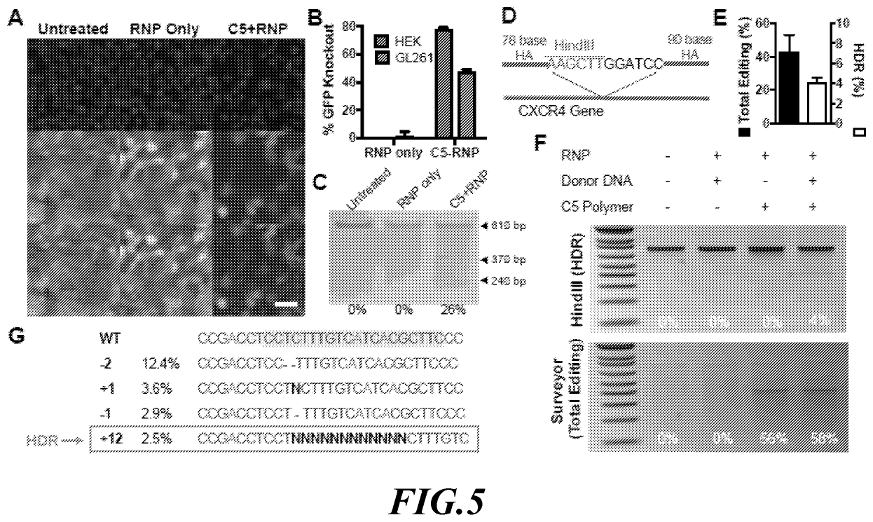

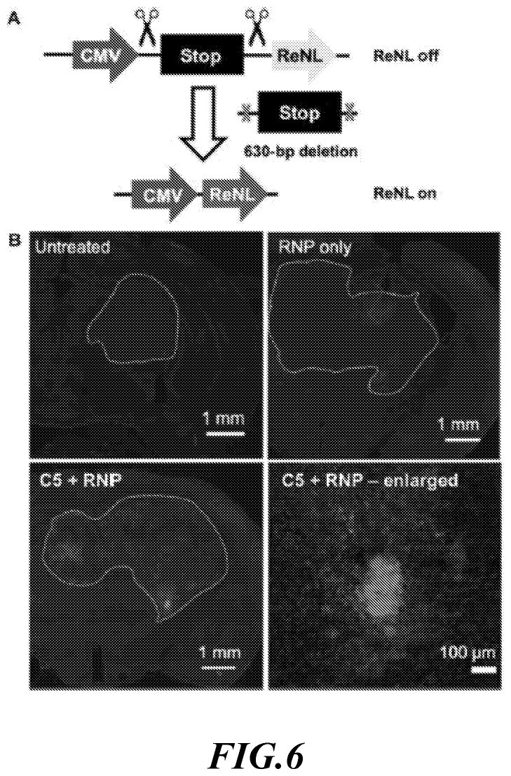

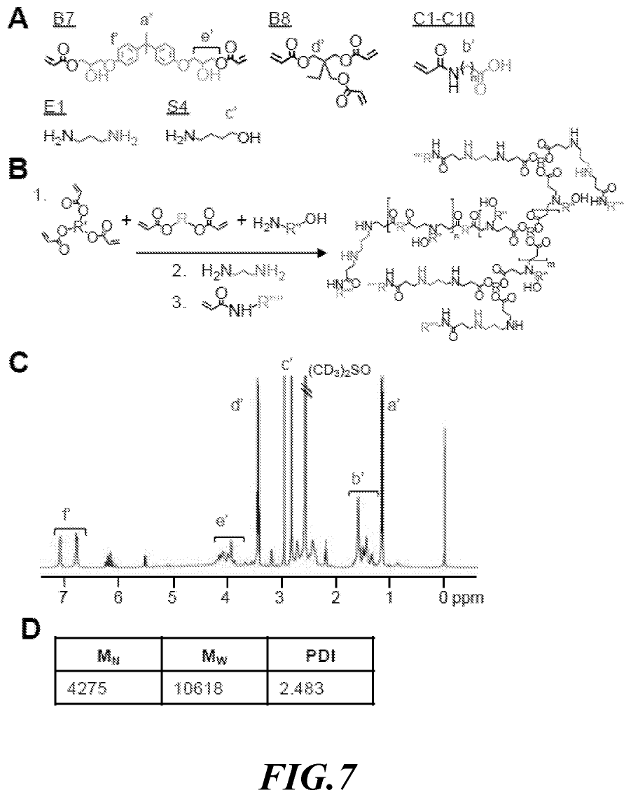

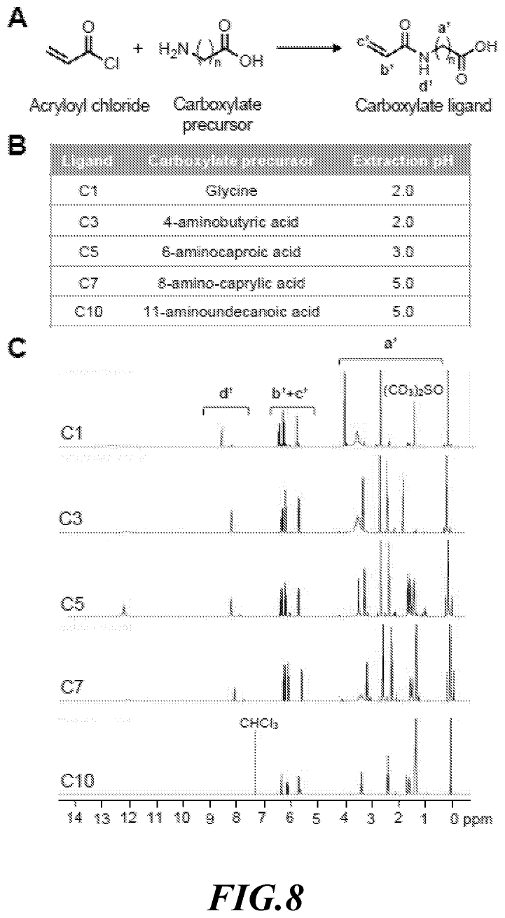

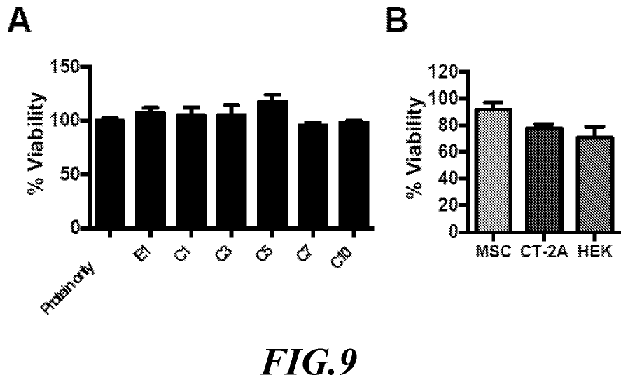

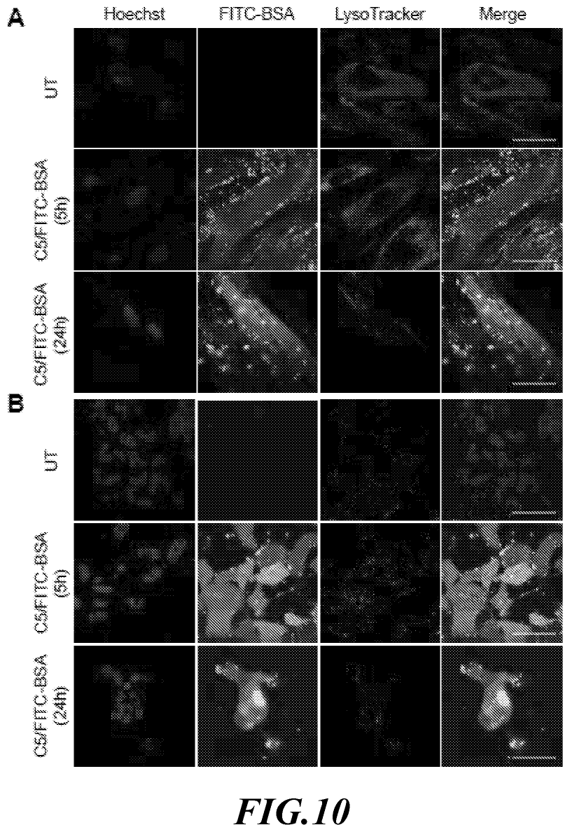

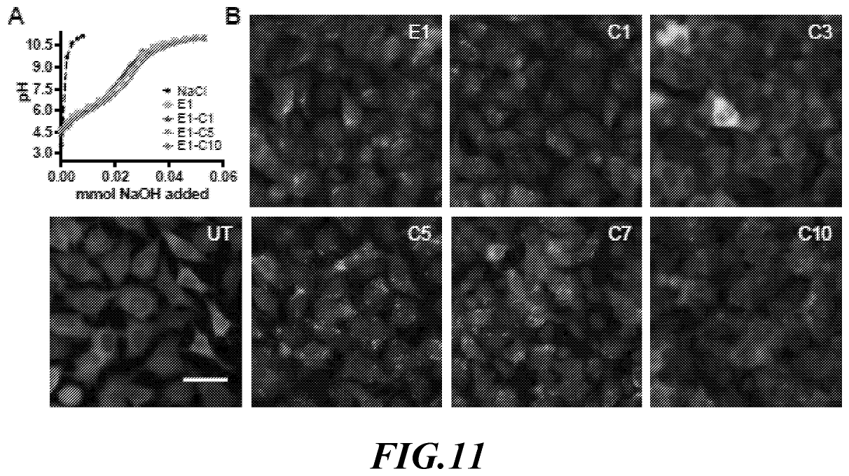

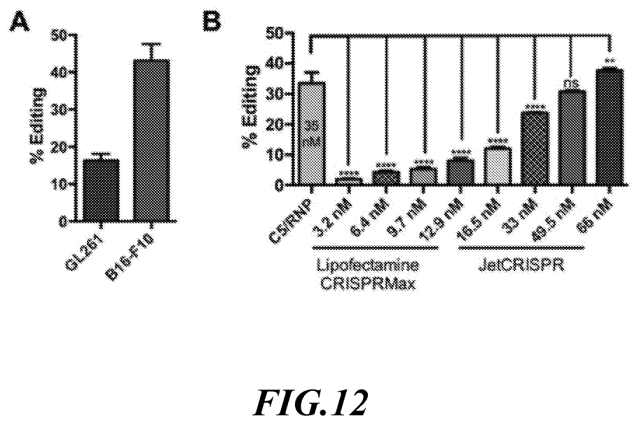

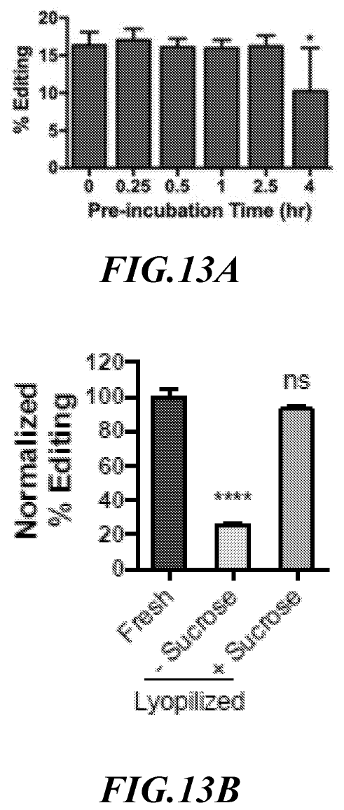

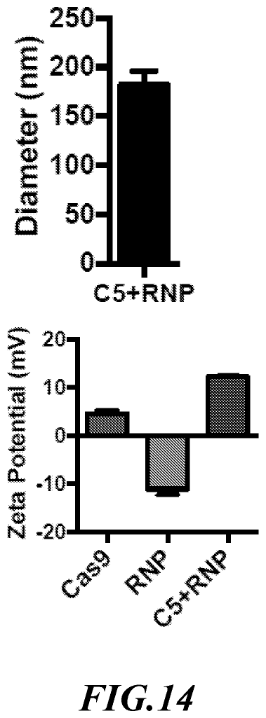

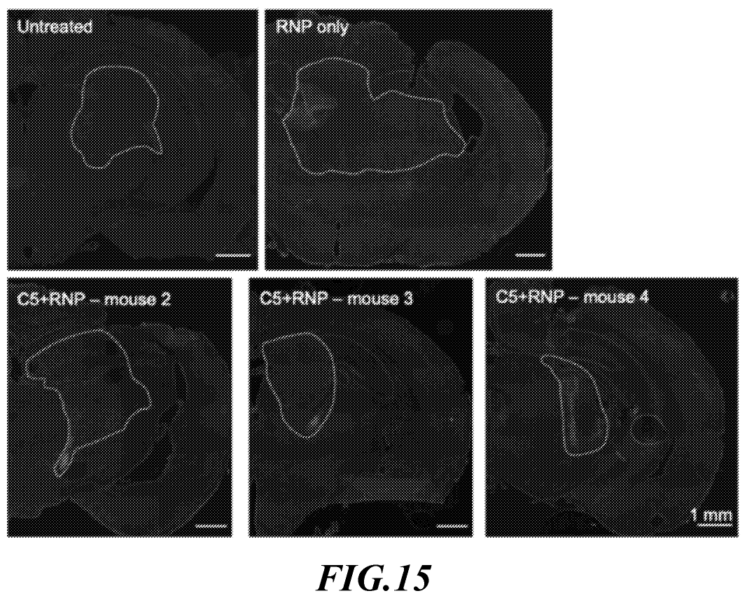

(FIG. 5A) Fluorescence microscopy of HEK-GFPd2 cells treated with RNPs alone or C5+RNPs; C5+RNPs enabled knockout of GFP fluorescence. Scale bar=50 μm. (FIG. 5B) Flow cytometry quantification of GFP knockout in HEK and GL261 cells. Data are mean+SEM; n=4. (FIG. 5C) Surveyor® mutation detector assay of GL261-GFPd2 cells treated with C5+RNP nanoparticles. (FIG. 5D) Experimental design of HDR assay in the CXCR4 gene; knock-in of a 12-bp insert flanked by homology arms (HA) results in the addition of a HindIII restriction enzyme site. (FIG. 5E) Quantification of total editing (via Surveyor® assay) and HDR (via HindIII restriction digest) in HEK cells. Data are mean+SEM; n=3. (FIG. 5F) HindIII restriction enzyme assay (top) and Surveyor® assay (bottom) of HEK cells treated with different C5/RNP/donor DNA combinations; orange arrow indicates HDR. (FIG. 5G) Inference of CRISPR Edits (ICE) analysis of Sanger sequencing data from C5+RNP+donor DNA treated cells provides a breakdown of different edits. Percentages indicate the percentage of the total DNA population with the indicated genotype. The targeted sequence is highlighted in grey and PAM sequence in yellow;

FIG. 6A and FIG. 6B demonstrate that C5/RNP nanoparticles enable CRISPR editing in vivo. (FIG. 6A) Schematic of CRISPR-stop gene construct; deletion of a 630-bp expression stop cassette turns on downstream ReNL expression. (FIG. 6B) Direct intracranial administration of C5/RNP nanoparticles to an orthotopic GL261-stop-ReNL tumor enabled CRISPR editing in vivo. Nanoparticles were formulated at 3.5-pmol RNP with C5 polymer (15 w/w). Tumor boundary is outlined in white;

FIG. 7A, FIG. 7B, FIG. 7C, and FIG. 7D show the synthesis and characterization of carboxylated branched PBAE polymers. (FIG. 7A) Monomer structures. (FIG. 7B) Reaction scheme for branched polymers. (1) Acrylate-terminated branched PBAE is synthesized via Michael addition of B and S monomers; (2) polymer endcapping with monomer E1 results in amine-terminated polymers; (3) further end-capping with carboxylate ligands yields final polymer products. (FIG. 7C) 1H NMR spectrum of polymer C5; distinctive peaks from each monomer are labeled according to chemical structures shown in (FIG. 7A). (FIG. 7D) Molecular weight data of polymer C5 obtained via GPC;

FIG. 8A, FIG. 8B, and FIG. 8C show the synthesis and characterization of carboxylate ligands. (FIG. 8A) Reaction route schematic. (FIG. 8B) Acidification pH for extraction of each ligand. (FIG. 8C) 1H NMR spectrum of each ligand; peaks labeled according to the chemical structure shown in (FIG. 8A);

FIG. 9A and FIG. 9B show cell viability after treatment with carboxylated branched PBAE protein nanoparticles. (FIG. 9A) Cell viability of CT-2A murine glioma cells treated with E1-C10 nanoparticles encapsulating BSA. (FIG. 9B) Cell viability of other cell types treated with C5/BSA nanoparticles. Nanoparticle formulation used in both experiments is 300 ng protein per well at 20 w/w;

FIG. 10A and FIG. 10B are confocal images of cells treated with C5/FITC-BSA nanoparticles. (FIG. 10A) Human adipose-derived mesenchymal stem cells and (FIG. 10B) HEK-293T cells were treated with C5/FITC-BSA nanoparticles (300 ng protein dose, 30 w/w) and imaged at 5 and 24 hours post-transfection. Untreated cells (UT) were also imaged as controls. Scale bar=10 μm. Data presented as mean+SEM; n=4;

FIG. 11A and FIG. 11B show characterization of polymer pH buffering and endosomal disruption capabilities. (FIG. 11A) pH titration curve of several polymers in the series. Sigmoidal curve fit of the titration curves and comparison via sum-of-squares F-test statistically demonstrated that there was no significant difference between buffering in the range of pH 4.5-8 for carboxylate ligands (P=0.062). (FIG. 11B) Representative images from high content imaging of B16-F10/Gal8-GFP cells treated with nanoparticles encapsulating BSA (125 ng BSA per well, 25 w/w; scale bar=50 μm);

FIG. 12A and FIG. 12B demonstrate that C5/RNP nanoparticles enable in vitro gene deletion. (FIG. 12A) C5/RNP deletion of stop cassette in vitro in 2 murine cancer cell lines resulted in turning on of ReNL fluorescence as detected by flow cytometry. Data are mean+SD; n=4. (FIG. 12B) Comparison in gene editing performance with commercially-available CRISPR transfection reagents in B16-F10 cells. C5/RNP nanoparticles were administered at an RNP dose of 35 nM per well; RNP dose for commercial reagents are indicated. For each commercial reagent, the manufacturer recommended RNP dose is indicated by checkerboard pattern. Turning on of ReNL fluorescence was quantified by flow cytometry. Data are mean+SD; n=4. Statistical analysis performed using one-way ANOVA with Dunnett's post-hoc tests compared against the C5/RNP group; **P<0.01, ****P<0.0001;

FIG. 13A and FIG. 13B demonstrate that C5/RNP nanoparticles are stable in serum-containing media and in lyophilized form. (FIG. 13A) % Editing observed in GL261-CRISPR-stop cells after treatment with nanoparticles pre-incubated in serum-containing complete medium at 37° C. for the designated times. Statistical significance determined by one-way ANOVA with Dunnett's post-hoc tests as compared to time=0. Data presented as mean+SD; (n=4). *P<0.05, ****P<0.0001;

FIG. 14A and FIG. 14B show representative RNP nanoparticle characterization (FIG. 14A) is the diameter (nm) of C5+RNP nanoparticles; and (FIG. 14B) is the Zeta potential (mV) of Cas9, RNP, and C5+RNP nanoparticles; and

FIG. 15 demonstrates that C5/RNP nanoparticle-enabled in vivo CRISPR editing is reproducible. Red ReNL fluorescent signal indicating CRISPR editing can be detected in the 3 additional mice treated with C5/RNP nanoparticles while untreated and RNP only groups showed no signal. Nanoparticles were formulated at 3.5 pmol RNP with 15 w/w C5 polymer. Tumors boundary outlined in white.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Polymeric Nanoparticles for Intracellular Protein Delivery

The presently disclosed subject matter provides a composition comprising a cationic core polymer, which preferably is biodegradable, having one or more anionic end groups, preferably having a functional group selected from a carboxylate, a phosphate, and a sulfonate, and more preferably having a carboxylate anionic end group, and their use for delivering one or more biomolecules, such as an amino-acid containing biomolecule, to a cell. In some embodiments, the cationic polymer comprises a naturally-derived cationic polymer. In certain embodiments, the naturally-derived cationic polymer is selected from the group consisting of chitosan, gelatin, dextran, cellulose, cyclodextrin, and a polypeptide, and/or other naturally-derived cationic polymers. In other embodiments, the cationic polymer comprises a synthetic cationic polymer. In certain embodiments, the synthetic cationic polymer is selected from the group consisting of a polyethyleneimine (PEI), poly-L-lysine (PLL), a poly(amidoamine) (PAA), a poly(amino-co-ester) (PAE), poly(2-N,N-dimethylaminoethylmethacrylate, a poly(beta-amino ester) (PBAE), an imidazole-containing polymer, a tertiary-amine containing polymer, poly(2-(dimethylamino)ethyl methacrylate), poly-N-(2-hydroxy-propyl)methacrylamide, polyamidoamine dendrimers, or derivatives thereof.

In some embodiments, the synthetic cationic polymer comprises a poly(beta-amino ester) (PBAE). Exemplary PBAEs suitable for use with the presently disclosed subject matter include those disclosed in: U.S. Pat. No. 9,884,118 for Multicomponent Degradable Cationic Polymers, to Green et al., issued Feb. 6, 2018; U.S. Pat. No. 9,802,984 for Biomimetic Peptide and Biodegradable Delivery Platform for the Treatment of Angiogenesis- and Lymphangiogenesis-Dependent Diseases, to Popel et al., issued Oct. 31, 2017; U.S. Pat. No. 9,717,694 for Peptide/Particle Delivery Systems, to Green et al., issued Aug. 1, 2017; U.S. Pat. No. 8,992,991 for Multicomponent Degradable Cationic Polymers, to Green et al., issued Mar. 31, 2015; U.S. Patent Application Publication No. 20180256745 for Biomimetic Artificial Cells: Anisotropic Supported Lipid Bilayers on Biodegradable Micro and Nanoparticles for Spatially Dynamic Surface Biomolecule Presentation, to Meyer et al., published Sep. 13, 2018; U.S. Patent Application Publication No. 20180112038 for Poly(Beta-Amino Ester)-Co-Polyethylene Glycol (PEG-PBAE-PEG) Polymers for Gene and Drug Delivery, to Green et al., published Apr. 26, 2018; U.S. Patent Application Publication No. 20170216363 for Nanoparticle Modification of Human Adipose-Derived Mesenchymal Stem Cells for Treating Brain Cancer and other Neurological Diseases, to Quinones-Hinojosa and Green, published Aug. 3, 2017; U.S. Patent Application Publication No. 20150273071 for Bioreducible Poly (Beta-Amino Ester)s For siRNA Delivery, to Green et al., published Oct. 1, 2015; U.S. Pat. No. 8,287,849 for Biodegradable Poly(beta-amino esters) and Uses Thereof, to Langer, et al., issued Oct. 16, 2012; each of which is incorporated by reference in their entirety.

In some embodiments, the anionic ligand end group is selected from the group consisting an amide-linked carboxylate, an ester-linked carboxylate, an ester-ethylene glycol-linked carboxylate, an amide-ethylene glycol-linked carboxylate, an ester-linked phosphate, an ester-ethylene glycol-linked phosphate, an amide-ethylene glycol-linked phosphate, an ester-linked sulfonate, and an ester-ethylene glycol-linked sulfonate, an amide-ethylene glycol-linked sulfonate.

In particular embodiments, the anionic ligand end group is selected from one or more of the following:

| Carboxylate | Phosphate | Sulfonate |
|---|---|---|
| 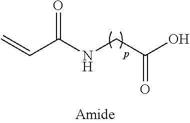 | 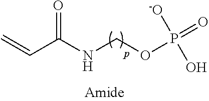 | 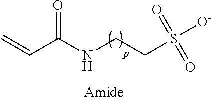 |
| 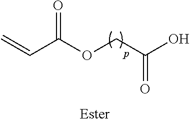 | 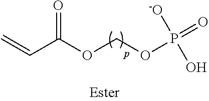 | 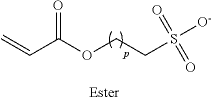 |
| 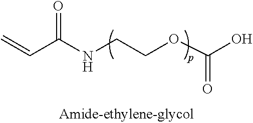 | 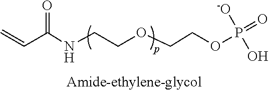 | 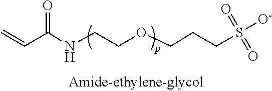 |
| 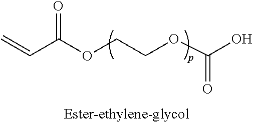 | 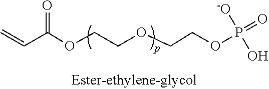 | 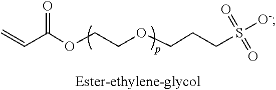 |

wherein p is an integer from 1, 2, 3, 4, 5, 6, 7, 8, 9, and 10.

In yet more particular embodiments, the presently disclosed subject matter provides modified branched PBAEs terminated with amino acid-like carboxylate ligands and their use for intracellular protein delivery. Without wishing to be bound to any one particular theory, it is thought that the carboxylated end-caps of the branched PBAEs could facilitate protein encapsulation through hydrogen bonding and salt bridges, while the PBAE polymer backbone could enable endosomal escape, thereby resulting in a versatile protein delivery platform.

In representative embodiments, carboxylate ligands were synthesized via acrylation of amino acid derivatives to yield a series of acrylated amino acids with varying numbers of carbon atoms between the carboxyl and amide groups (note that ligands are referred to by the number of carbon atoms, e.g., C1, C2, C3, C4, C5, C6, C7, C8, C9, and C10, between the carboxyl and amide groups, with C1 corresponding to glycine). Amine-terminated PBAEs were synthesized via a Michael addition reaction and then end-capped with carboxylate ligands. Protein-encapsulated nanocomplexes were formed by mixing polymer and proteins under conditions to facilitate nanoparticle self-assembly.

The presently disclosed carboxylated branched polymers complex with proteins to form nanoparticles between about 100 nm to about 600 nm in diameter, including about 100 nm, 125 nm, 150 nm, 175, nm 200 nm, 225 nm, 300 nm, 325 nm, 350 nm, 375 nm, 400 nm, 425 nm, 450, nm, 475 nm, 500 nm, 525 nm, 550 nm, 575 nm and 600 nm, with each nanoparticle containing between about 1-1000 protein molecules. Generally, protein uptake exhibited a biphasic response relative to the number of carbons in the carboxylate end-cap, with C5 and C7 ligands achieving the highest levels of uptake. More particularly, characterization of a series of carboxylated branched PBAEs demonstrated that polymers having C5 end-groups provided a high level of cytosolic protein delivery in multiple cell types. For example, cytosolic delivery of saporin induced highly effective cell killing and delivery of Cas9 RNPs enabled CRISPR gene knockout up to about 80%.

Overall, in representative embodiments as provided in more detail herein below, the presently disclosed carboxylated branched PBAEs facilitate protein delivery independent of protein surface charge; provide ribonucleoprotein (RNP) delivery for CRISPR/Cas9 editing; deliver protein to cells in complete 10% serum-containing growth medium; and deliver protein or gene edit a wide-variety of adherent cell types. Accordingly, the presently disclosed carboxylated branched PBAEs are a versatile protein delivery platform and a promising tool for gene editing applications.

### A. Carboxylated Branched Poly(β-Amino Ester) Protein Nanoparticles

Poly(β-amino ester)s (PBAEs) are cationic, biodegradable polymers synthesized via the following Michael addition reaction:

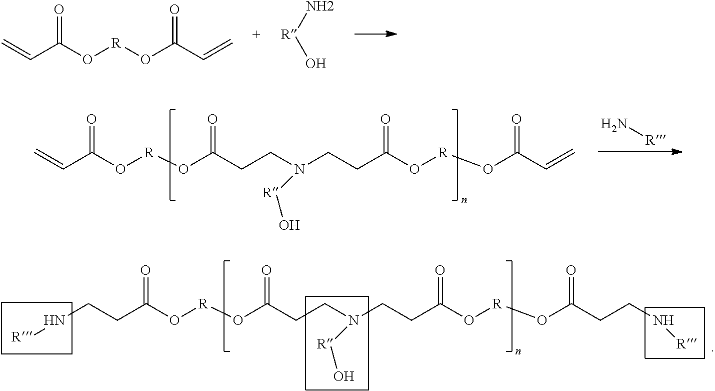

In representative PBAEs, as shown immediately hereinabove, the cationic secondary amines (designated R″′) in the end-capping groups can bind a nucleic acid; the titratable tertiary amines (designated R″) in the side chain facilitate endosomal escape; and the hydrolysable ester bonds in the backbone (designated R) facilitate cargo release and attenuate vector toxicity. Such PBAEs have been shown to successfully delivery plasmid DNA to a wide range of cell types. See Tzeng et al., ACS Nano (2015); Mangraviti et al., Biomaterials (2016).

In contrast to PBAEs known in the art for nucleic acid delivery, the presently disclosed subject matter provides PBAE's exhibiting polymer branching and having carboxylate end-groups for protein delivery. A representative schematic of the preparation of such PBAE's is provided herein below:

wherein S4 is a titratable tertiary amine side chain monomer designated as (R″) hereinabove; B7 is a backbone polymer having a hydrolysable ester bond designated as (R) hereinabove; B8 is a branched polymer having a hydrolysable ester bond; and E1 is a cationic secondary amine end-capping group designated as (R′″) hereinabove.

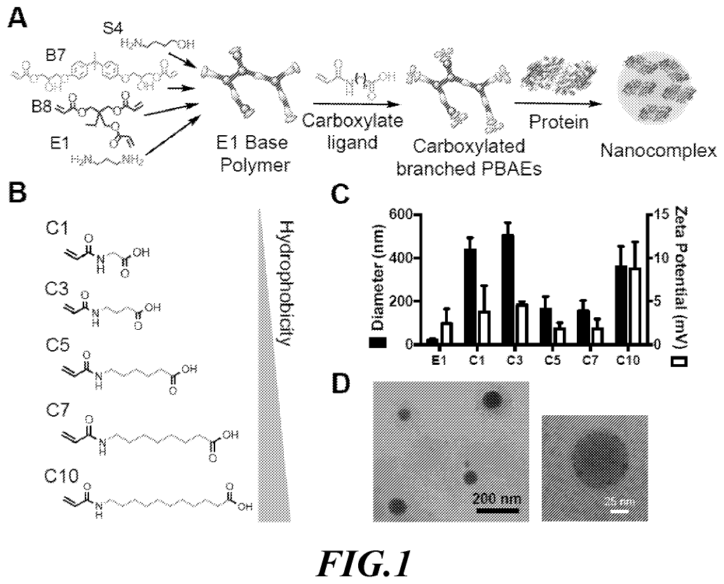

Representative monomers suitable for use in preparing the presently disclosed PBAE branched polymer are provided hereinbelow. Such monomers can be used to prepare a so-called “base polymer” having a cationic secondary amine end-capping group, i.e., an “E” monomer, such as “E1.” The base polymer can be reacted with a carboxylate ligand to form a carboxylated branched PBAE. See also FIG. 1A and FIG. 1B. Representative carboxylate ligands are provided immediately herein below:

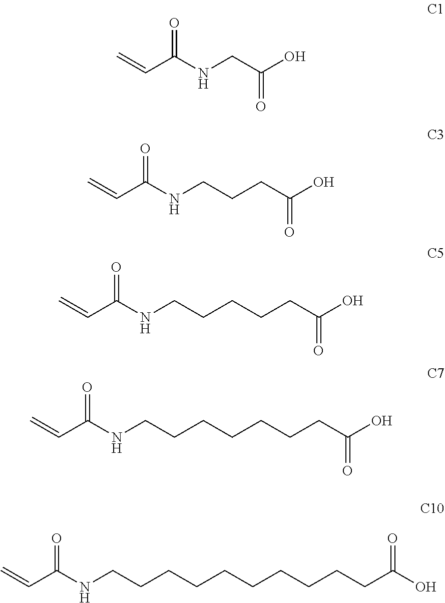

Base branched PBAE polymers suitable for use in preparing the presently disclosed carboxylated branched PBAE protein nanoparticles are described in U.S. provisional patent application No. 62/743,883, which is incorporated herein by reference in its entirety.

Generally, such PBAEs are biodegradable, e.g., they degrade in water or an aqueous solution. In certain embodiments the degradation is pH-dependent. In particular embodiments, the particles comprise branched PBAE polymers having a backbone constructed from diacrylate monomers in combination with triacrylate monomers to provide polymers with variable branching. The polymers can be prepared by condensing side chain monomers comprising secondary amines or primary amines with acrylate ester monomers, e.g., diacrylate and triacrylate monomers. For example, in some embodiments, the PBAE comprises a backbone of a diacrylate, e.g., bisphenol A glycerolate (1 glycerol/phenol) diacrylate (BGDA), and a triacrylate, e.g., trimethylolpropane triacrylate (TMPTA).

In some embodiments, the polymers comprise tertiary amines in their backbone and/or in some embodiments, the polymers comprise side chains and/or end groups comprising primary, secondary, and/or tertiary amines. In some embodiments, the secondary or tertiary amines comprise bivalent amine-containing heterocyclic groups. In some embodiments, the side chain monomers comprise a primary amine, but also may comprise secondary and tertiary amines. In some embodiments, the end group terminates with a primary amine and a hydroxyl, with an internally placed secondary amine.

More particularly, in some embodiments, the presently disclosed subject matter provides compositions, including particles, comprising carboxylated branched PBAEs for delivery proteins to cells. The presently disclosed polymers have the property of biphasic degradation and modifications to the polymer structure can result in a change in the release of therapeutic agents, e.g., a protein. In some embodiments, the presently disclosed polymers include a minority structure, e.g., an endcapping group, which differs from the majority structure comprising most of the polymer backbone. In other embodiments, the bioreducible oligomers form block copolymers with hydrolytically degradable oligomers. In yet other embodiments, the end group/minority structure comprises an amino acid or chain of amino acids, or, in particular embodiments, carboxylate ligands synthesized via acrylation of amino acid derivatives to yield a series of acrylated amino acids with varying numbers of carbon atoms between the carboxyl and amide groups, while the backbone degrades hydrolytically and/or is bioreducible.

Small changes in the monomer ratio used during polymerization, in combination with modifications to the chemical structure of the end-capping groups used post-polymerization, can affect the efficacy of delivery of a protein to a cell. Further, changes in the chemical structure of the polymer, either in the backbone of the polymer or end-capping groups, or both, can change the efficacy of protein delivery to a cell. In some embodiments, small changes to the molecular weight of the polymer or changes to the endcapping groups of the polymer, while leaving the main chain, i.e., backbone, of the polymer the same, can enhance or decrease the overall delivery of the protein to a cell. Further, the “R” groups that comprise the backbone or main chain of the polymer can be selected to degrade via different biodegradation mechanisms within the same polymer molecule. Such mechanisms include, but are not limited to, hydrolytic, bioreducible, enzymatic, and/or other modes of degradation.

The properties of the presently disclosed carboxylated, branched PBAEs can be tuned to impart one or more of the following characteristics to the composition: independent control of cell-specific uptake and/or intracellular delivery of a particle; independent control of endosomal buffering and endosomal escape; independent control of protein release; triggered release of an active agent; modification of a particle surface charge; increased diffusion through a cytoplasm of a cell; increased active transport through a cytoplasm of a cell; increased nuclear import within a cell; and/or increased persistence of an associated therapeutic agent within a cell.

If a hydrophilic peptide/protein is to be encapsulated, a hydrophilic polymer is chosen as the multicomponent material. If a hydrophobic peptide/protein is to be encapsulated than a hydrophobic polymer is chosen. The polymer backbone, side chain, and/or terminal group can be modified to increase the hydrophobic or hydrophilic character of the polymer. The peptide/protein to be encapsulated can be first dissolved in a suitable solvent, such as DMSO or PBS. Then, it is combined with the polymer in, for example, sodium acetate (NaAc). This solution is then diluted with either sodium acetate, OptiMem, DMEM, PBS, or water depending on the particle size desired. The solution in vortexed to mix and then left to incubate for a period of time for particle assembly to take place. The particles can self-assemble with a protein to form nanoparticles that can be in the range of 50 nm to 500 nm in size.

Representative multicomponent degradable cationic polymers are disclosed in the following U.S. patents and U.S. patent application publications, each of which is incorporated herein by reference in its entirety: U.S. Patent Application Publication No. 20180177881 for Multicomponent Degradable Cationic Polymers, to Green et al., published Jun. 28, 2018; U.S. Patent Application Publication No. 20150250881 for Multicomponent Degradable Cationic Polymers, to Green et al., published Sep. 10, 2015; U.S. Patent Application Publication No. 20120128782 for Multicomponent Degradable Cationic Polymers, to Green et al., published May 24, 2012; U.S. Patent Application Publication No. 20180112038 for Poly(beta-amino ester)-co-polyethylene glycol (PEG-PBAE-PEG) Polymers for Gene and Drug Delivery, to Green et al., published Apr. 26, 2018; U.S. Patent Application Publication No. 20180028455 for Peptide/Particle Delivery Systems, to Green et al., published Feb. 1, 2018; U.S. Patent Application Publication No. 20160374949 for Peptide/Particle Delivery Systems, to Green et al., published Dec. 29, 2016; U.S. Patent Application Publication No. 20120114759 for Peptide/Particle Delivery Systems, to Green et al., published Dec. 29, 2016; U.S. Patent Application Publication No. 20160122390 for A Biomimetic Peptide and Biodegradable Delivery Platform for the Treatment of Angiogenesis- and Lymphangiogenesis-Dependent Diseases, to Popel, et al, published May 5, 2016; U.S. Patent Application Publication No. 20150273071 for Bioreducible Poly (Beta-Amino Ester)s for siRNA Delivery, to Green et al., published Oct. 1, 2015; U.S. Pat. No. 9,884,118 for Multicomponent Degradable Cationic Polymers, to Green, et al., issued Feb. 6, 2018; U.S. Pat. No. 9,717,694 for Peptide/particle Delivery Systems, Green, et al., issued Aug. 1, 2017; and U.S. Pat. No. 8,992,991 for Multicomponent Degradable Cationic Polymers, to Green, et al., issued Mar. 31, 2015; U.S. Pat. No. 8,287,849 for Biodegradable Poly(beta-amino esters) and Uses Thereof, to Langer, et al., issued Oct. 16, 2012. Other exemplary PBAE polymers are described in WO/2012/0128782, WO/2012/0114759, WO/2014/066811, WO/2014/066898, and US2016/0122390, each of which is incorporated herein by reference in its entirety. In some embodiments, the presently disclosed particles can comprise a polymer blend of PBAEs, e.g., a mixture of PBAE polymers.

Generally, the presently disclosed PBAEs include a backbone derived from a diacrylate monomer (designated herein below as “B”), an amino-alcohol side chain monomer (designated herein below as “S”), and an amine-containing end-cap monomer (designated herein below as “E”). The end group structures are distinct and separate from the polymer backbone structures and the side chain structures of the intermediate precursor molecule for a given polymeric material. The presently disclosed PBAE compositions can be designated, for example, as B5-S4-E7 or 547, in which R is B5, R″ is S4, and R′″ is E7, and the like, where B is for backbone and S is for the side chain, followed by the number of carbons in their hydrocarbon chain. Endcapping monomers, E, are sequentially numbered according to similarities in their amine structures.

In particular embodiments, the presently disclosed polymers have a backbone constructed from a triacrylate monomer to provide polymers with variable branching.

More particularly, in some embodiments, the presently disclosed subject matter provides a composition comprising a branched poly(beta-amino ester) (PBAE) of formula (I) or formula (II):

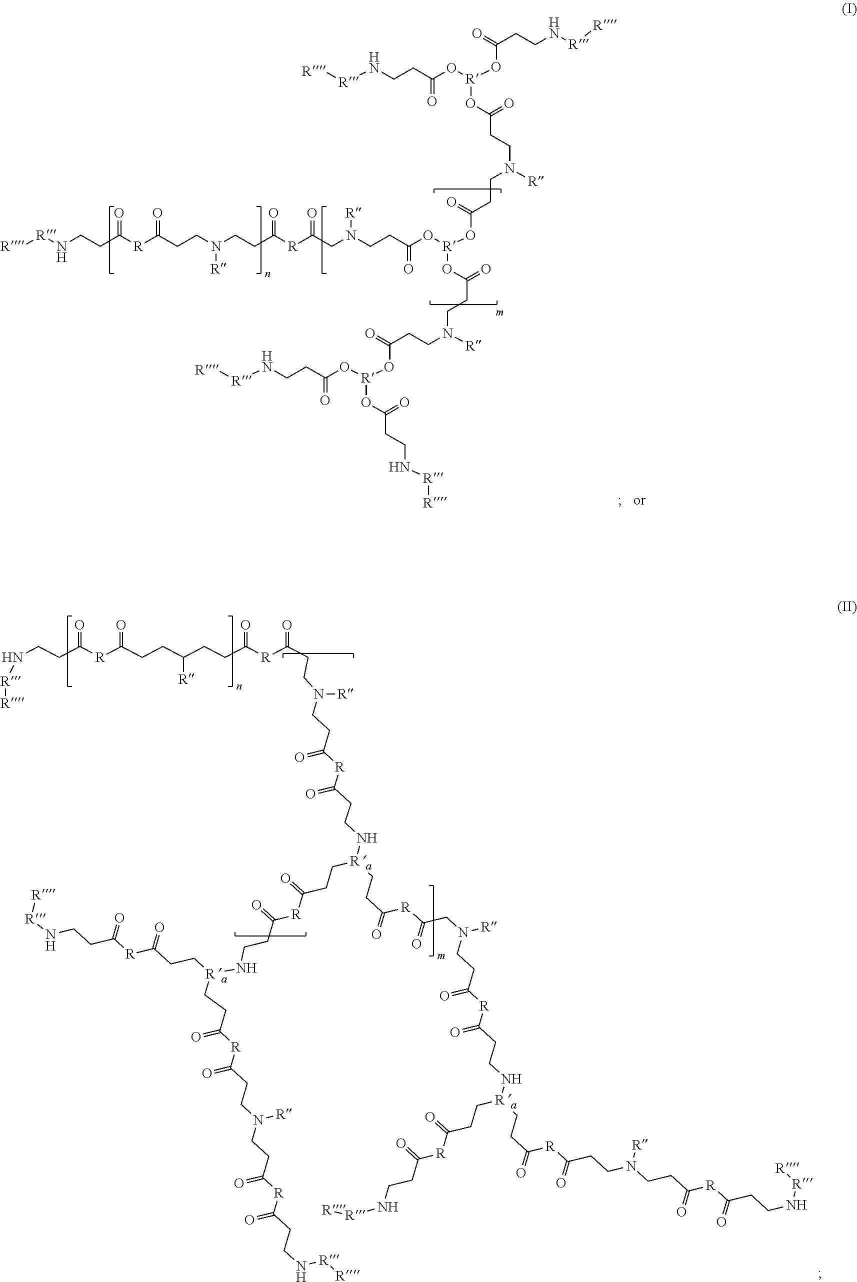

wherein: n and m are each independently an integer from 1 to 10,000; each R is independently a diacrylate monomer of the following structure:

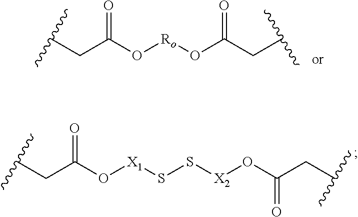

wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain;
each R′ of formula (I) is a trivalent group of a triacrylate monomer having the following structure:

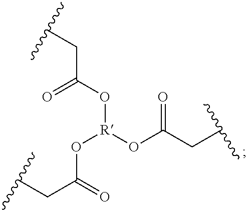

each R′ₐ of a compound of formula (II) is a tri-functional amine (e.g., NH₂—R′ₐ);
each R″ is independently a side chain monomer comprising a primary, secondary, or tertiary amine; each R′″ is independently an end group monomer comprising a primary, secondary, or tertiary amine; each R″″ is independently an anionic end group; and pharmaceutically acceptable salts thereof.

Embodiments comprising the composition of formula (II), which has a tri-functional amine as the linking branching unit, can be prepared as follows:

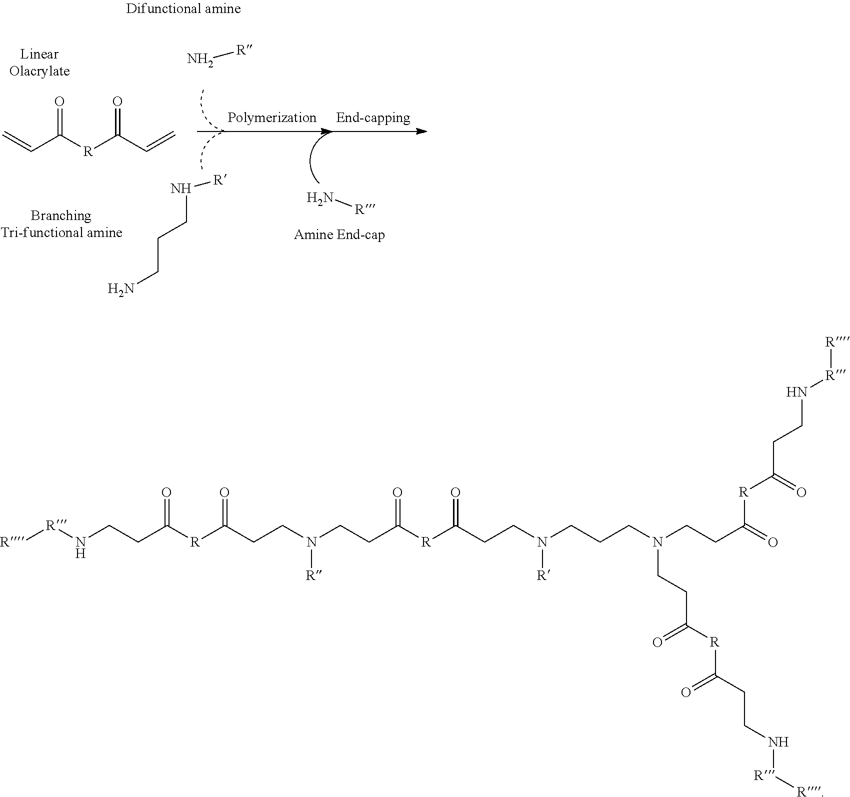

One of ordinary skill in the art would recognize that any amine-containing monomer with either one primary and one secondary or three secondary amines would be suitable for use as the branching tri-functional amine with the presently disclosed compositions of formula (II).

In some embodiments of the composition of formula (I) or formula (II), R is selected from the group consisting of:

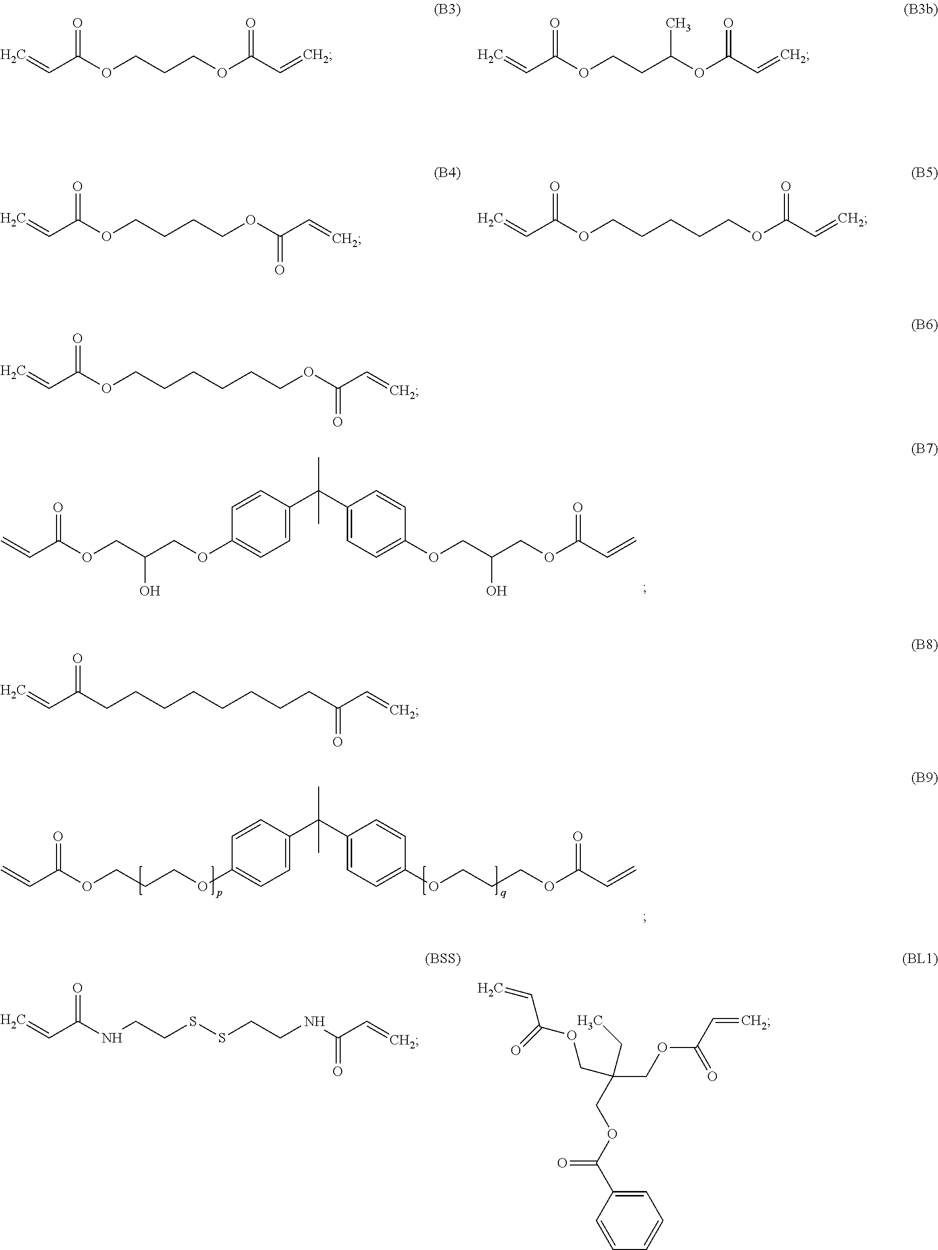

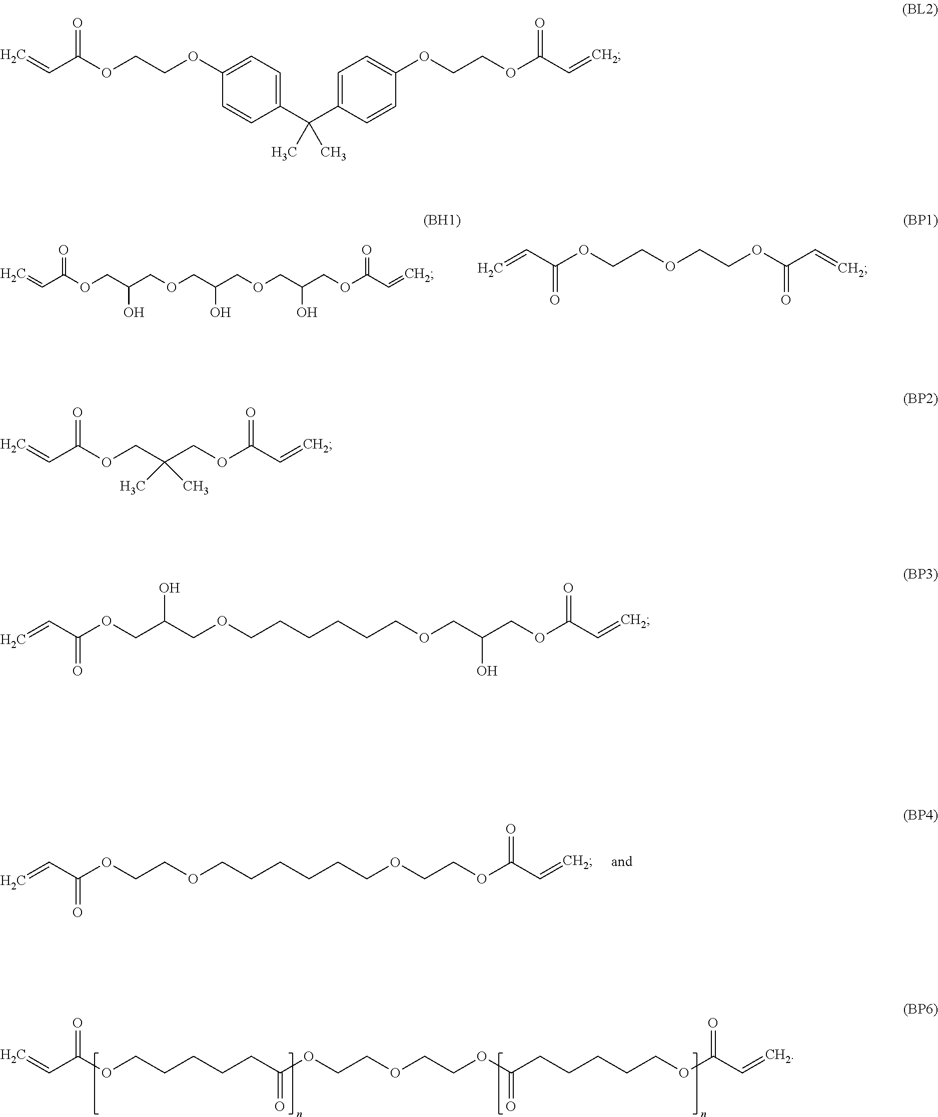

In some embodiments of the composition of formula (I), the trivalent group R′ is —C—CH₂CH₃ and the triacrylate monomer is trimethylolpropane triacrylate (TMPTA):

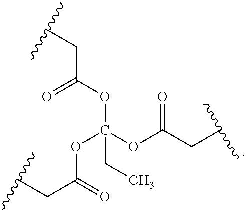 [SMILES: *CC(=O)OC(CC)(OC(=O)C*)OC(=O)C*]

In other embodiments of the composition of formula (II), the tri-functional amine monomer is selected from the group consisting of:

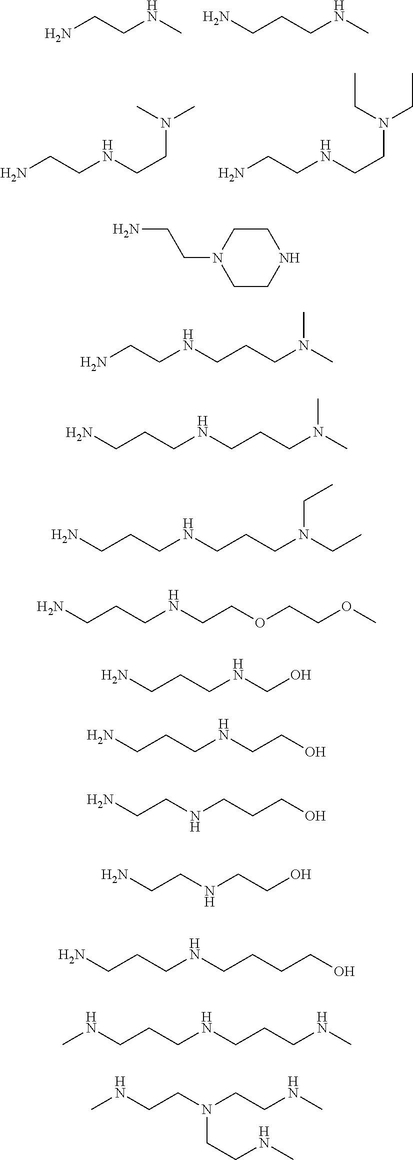

In some embodiments of the composition of formula (I) or formula (II), R″ is selected from the group consisting of:

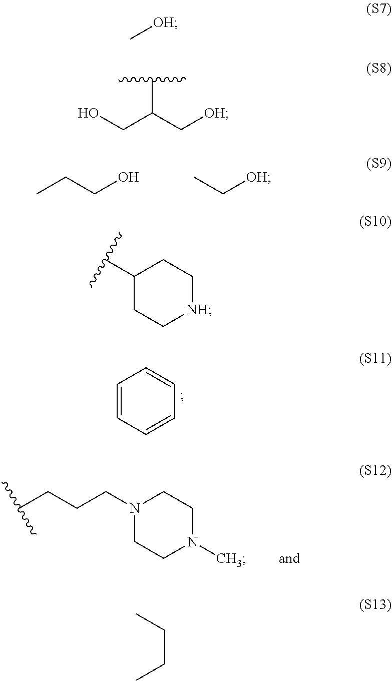

In other embodiments of the composition of formula (I) or formula (II), R″ is selected from the group consisting of:

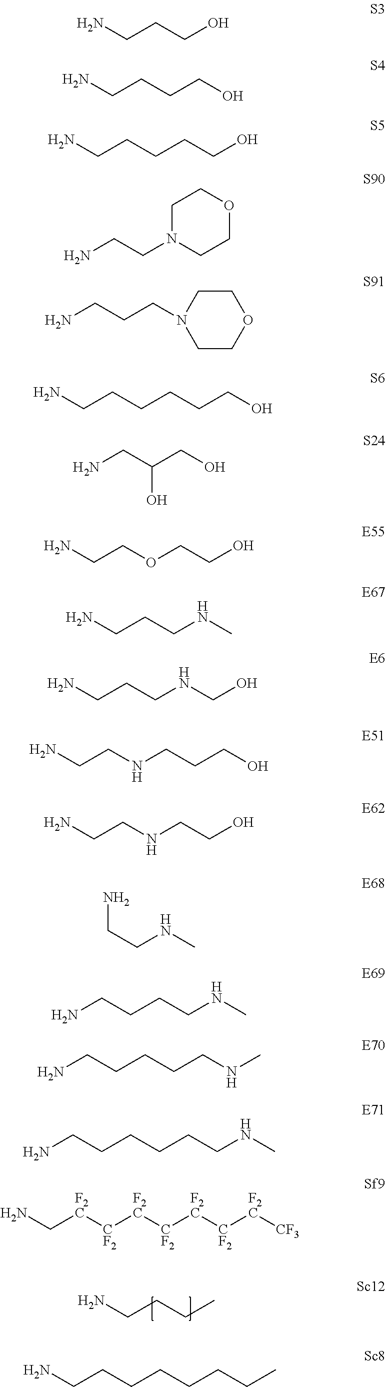

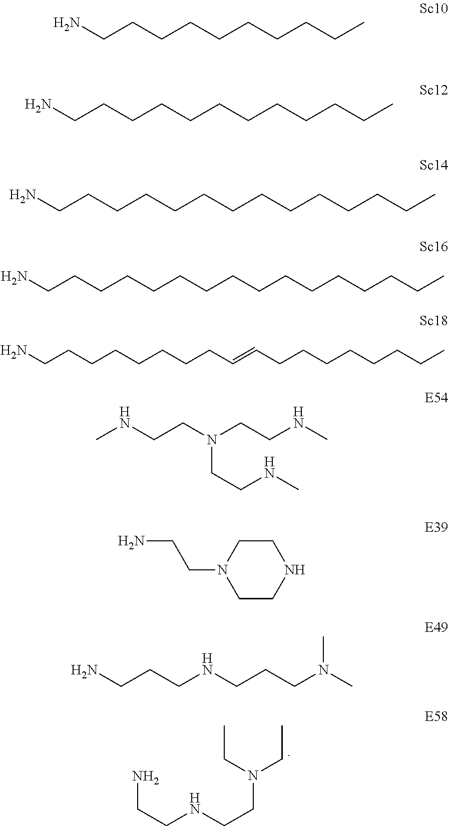

In some embodiments of the composition of formula (I) or formula (II), R′″ is selected from the group consisting of:

Amino Alkanes

| | |
|---|---|
| 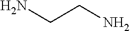 | A1 |
| 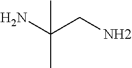 | A2 |
| 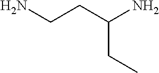 | A3 |
| 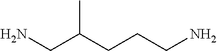 | A4 |
| 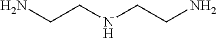 | A5 |
| 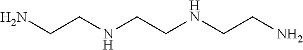 | A6 |
| 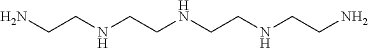 [SMILES: NCCNCCNCCNCCN] | A7 |
| 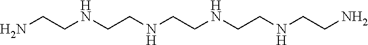 [SMILES: NCCNCCNCCNCCNCCN] | A8 |
| 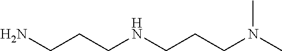 | A9 |
| 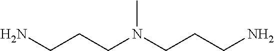 | A10 |
| 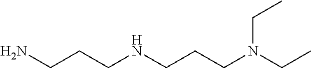 | A11 |
| 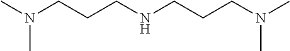 | A12 |
| 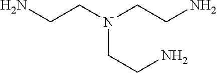 | A13 |
| 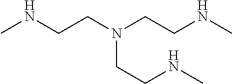 | A14 |

Amino Piperidines

| | |
|---|---|
| 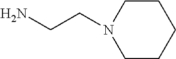 | B1 |
| 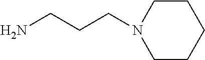 | B2 |
| 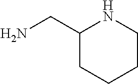 | B3 |
| 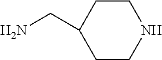 | B4 |

Amino Pyrrolidines

| | |
|---|---|
| 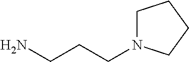 | D1 |
| 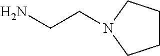 | D2 |

Amino Alcohols

| | |
|---|---|
| 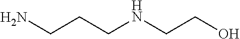 | E1 |
| 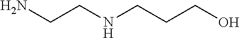 | E2 |
| 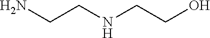 | E3 |
| 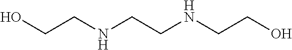 | E4 |
| 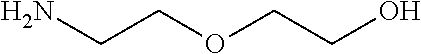 | E5 |
| 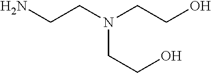 | E6 |

Amino Piperizines

| | |
|---|---|
| 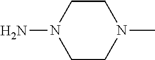 | C1 |
| 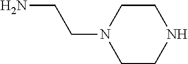 | C2 |
| 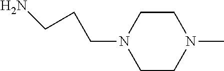 | C3 |
| 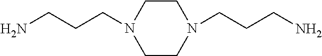 [SMILES: NCCCN1CCN(CCCN)CC1] | C4 |

Diamino ethers

| | |
|---|---|
| 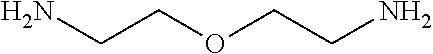 | F1 |
| 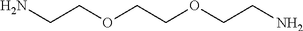 | F2 |
| 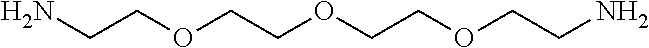 [SMILES: NCCOCCOCCOCCN] | F3 |
| 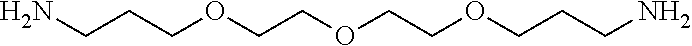 [SMILES: NCCCOCCOCCOCCCN] | F4 |

Amino morpholinos

| | |
|---|---|
| 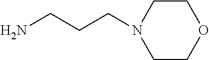 | G1 |
| 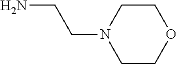 | G2 |

In other embodiments of the composition of formula (I) or formula (II), R′″ is selected from the group consisting of:

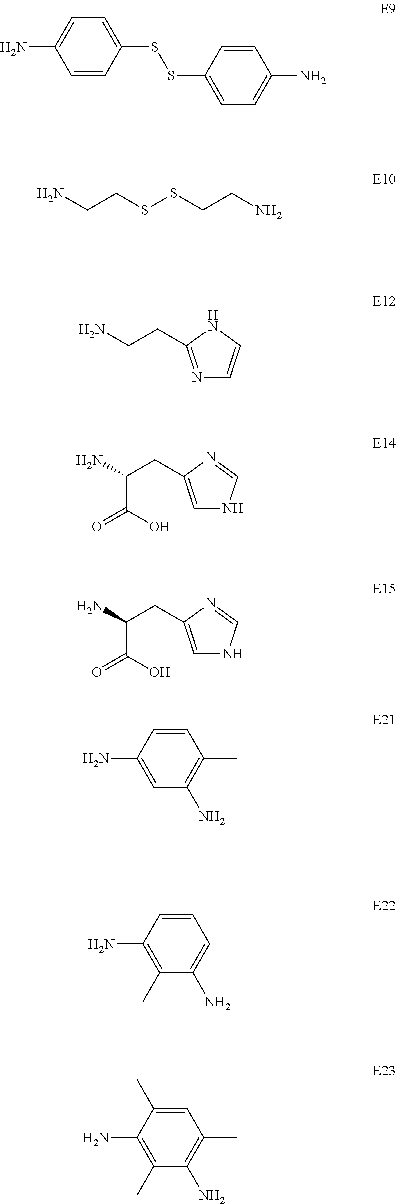

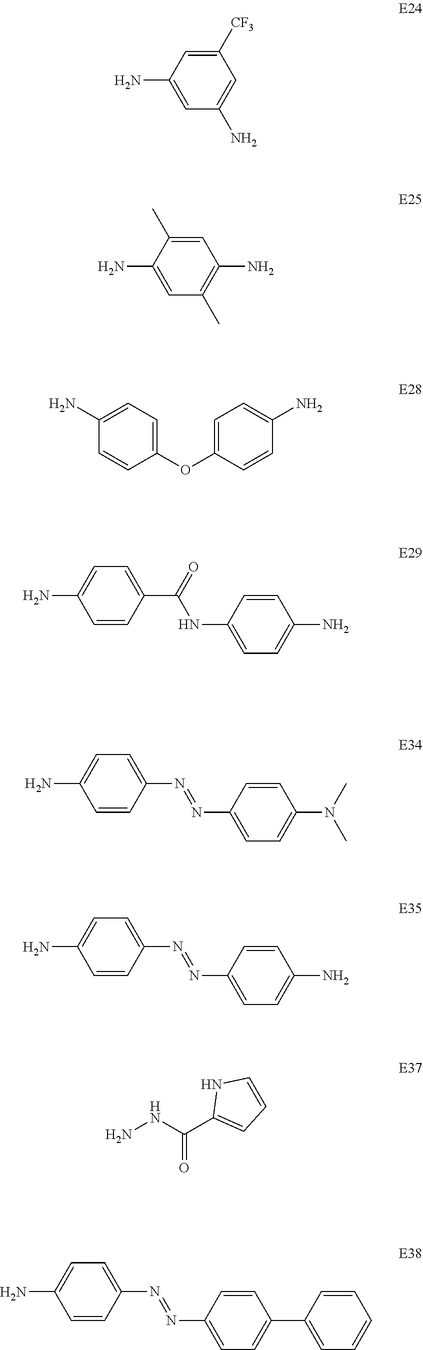

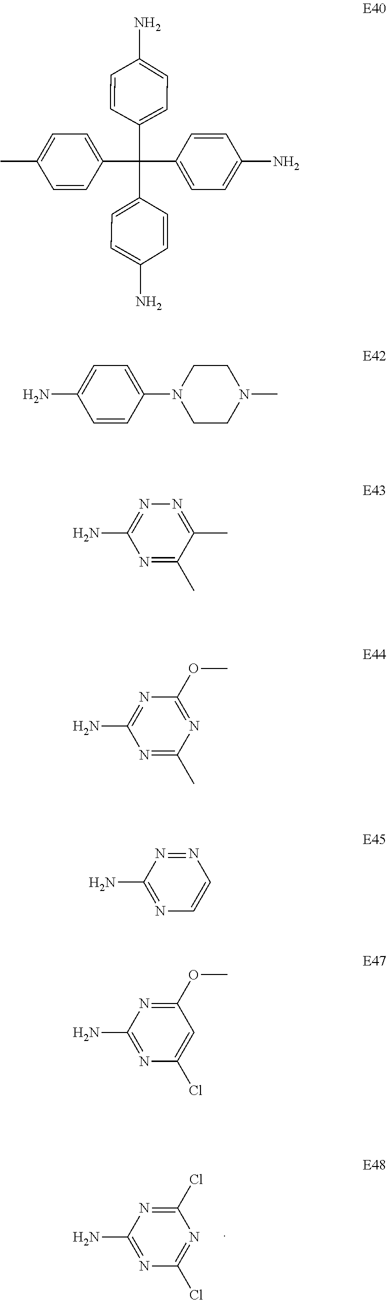

In some embodiments of the composition of formula (I) or formula (II), R″″ is selected from the group consisting of:

| Carboxylate Anionic ligands R′′′′ | Phosphate Anionic ligands R′′′′ | Sulfate Anionic ligands R′′′′ |
|---|---|---|
| 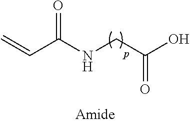 | 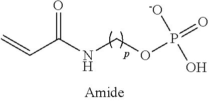 | 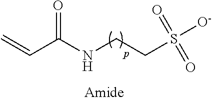 |
| 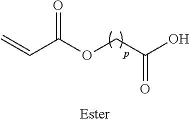 | 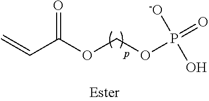 | 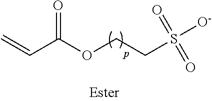 |
| 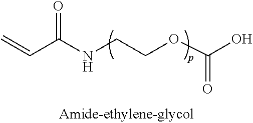 | 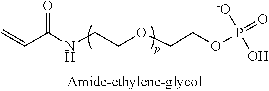 | 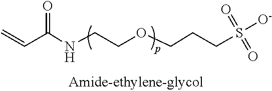 |
| 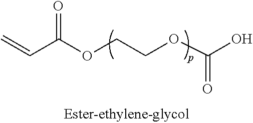 | 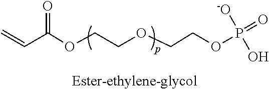 | 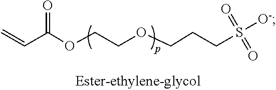 |

wherein p is an integer from 1, 2, 3, 4, 5, 6, 7, 8, 9, and 10.

In particular embodiments of the composition of formula (I) or formula (II), R″″ is:

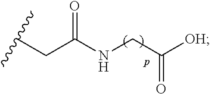

wherein p is an integer from 1, 2, 3, 4, 5, 6, 7, 8, 9, and 10.

In yet more particular embodiments of the composition of formula (I) or formula (II), R″″ is selected from the group consisting of:

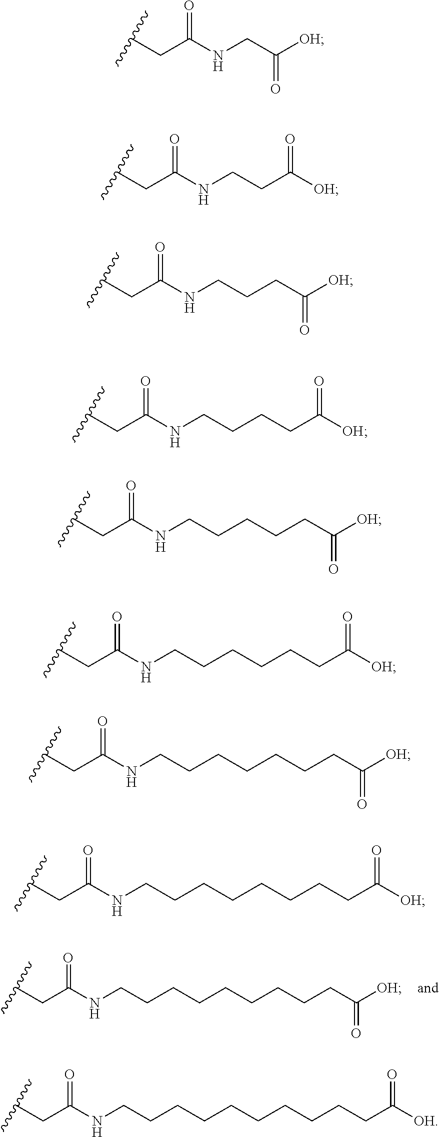

In certain embodiments of a composition of formula (I) or formula (II), n and m are each independently selected from the group consisting of: an integer from 1 to 1,000; an integer from 1 to 100; an integer from 1 to 30; an integer from 5 to 20; an integer from 10 to 15; and an integer from 1 to 10.

In some embodiments, the presently disclosed composition further comprises one or more biomolecules. In certain embodiments, the composition further comprises one or more biomolecules selected from a peptide, a protein, a nucleic acid, a morpholino, and other charged or zwitterionic biomolecules or combinations thereof. In more certain embodiments, the one or more biological molecules is selected from the group consisting of a non-peptide based biological small molecule or a biomacromolecule selected from a sugar, a polysaccharide, a carbohydrate, a morpholino oligomer, and/or a nucleic acid. In more certain embodiments, the one or more biomolecules comprises a protein.

In particular embodiments, the protein is selected from the group consisting of a ribosome inactivating protein (RIP), a gene-editing protein, an immunoglobulin, a nanobody, and an intrabody. In more particular embodiments, the ribosome inactivating protein (RIP) is selected from the group consisting of abrin, beetin, ricin, saporin, Shiga toxin, a Spiroplasma protein, trichosanthin, and viscumin.

In certain embodiments, the protein comprises a gene editing protein. In more certain embodiments, the gene editing protein comprises a Cas9 ribonucleoprotein (RNP). In other embodiments, the composition further comprises a guide RNA (gRNA).

In other embodiments, the protein is labeled with one or more ligands suitable for detecting the protein in a cell. In particular embodiments, the label comprises a fluorescent label. In more particular embodiments, the fluorescent label is selected from the group consisting of fluorescein isothiocyanate (FITC), green fluorescent protein (GFP), AlexaFluor 350, AlexaFluor 430, AlexaFluor405, AlexaFluor488, AlexaFluor546, AlexaFluor555, AlexaFluor594, AlexaFluor660, AlexaFluor633, AlexaFluor647, AlexaFluor680, AlexaFluor700, AlexaFluor750, AlexaFluor790, AMCA, (BODIPY) dye, or derivatives thereof, including, but not limited to, BODIPY 630/650, BODIPY 650/665, BODIPY 581/591, BODIPY-FL, BODIPY-R6G, BODIPY-TR, BODIPY-TMR, BODIPY-TRX, Cascade Blue, Cy3, Cy5, Cy5.5, Cy7, 6-FAM, fluorescein, Fluorescein Isothiocyanate, TRITC, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, Texas Red, carbocyanine, indocarbocyanine, oxacarbocyanine, thuicarbocyanine, merocyanine, polymethine, coumarine, rhodamine, xanthene, a boron-dipyrromethane VivoTag-680, VivoTag-S680, VivoTag-S750, Dy677, Dy676, Dy682, Dy752, Dy780, DyLight547, DyLight647, DyLight 350 (Ex/Em=353 nm/432 nm), DyLight 405 (400/420), DyLight 488 (493/518), DyLight 550 (562/576), DyLight 594 (593/618), DyLight 633 (638/658), DyLight 650 (652/672), DyLight 680 (692/712), DyLight 755 (754/776), DyLight 800 (777/794), and derivatives thereof, including, but not limited to, NHS esters, maleimides, phosphines, and free acids, HiLyte Fluor 647, HiLyte Fluor 680, HiLyte Fluor 750, IR800 (EHmethyl{4-[1,5,5-tris(4-dimethylaminophenyl)-2,4-pentadienylidene]-2,5-cyclohexadien-1-ylidene} ammonium perchlorate), IRDye 800CW, IRDye 800RS, IRDye 700DX, ADS780WS, ADS830WS, ADS832WS, R-Phycoerythrin, Flamma749, Flamma774, and ICG.

In certain embodiments, the branched carboxylated PBAE polymer has a molecular weight of from 5 to 10 kDa, or a molecular weight of from 10 to 15 kDa, or a molecular weight of from 15 to 25 kDa, or a molecular weight of from 25 to 50 kDa.

In certain embodiments, the presently disclosed subject matter provides a pharmaceutical formulation of comprising the PBAE composition of formula (I) or formula (II) in a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

In particular embodiments, the pharmaceutical formulation further comprises one or more therapeutic agents.

In yet other embodiments, the pharmaceutical formulation further comprises a nanoparticle or microparticle of the PBAE of formula (I) or formula (II). The PBAE polymers in some embodiments can self-assemble with a protein, to form nanoparticles which may be in the range of 50 to 500 nm in size, e.g., about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm in size.

In embodiments, the particle has at least one dimension in the range of about 50 nm to about 500 nm, or from about 50 to about 200 nm. Exemplary particles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo.

In some embodiments, the presently disclosed particles may comprise other combinations of cationic polymeric blends or block co-polymers. Additional polymers include polycaprolactone (PCL), polyglycolic acid (PGA), polylactic acid (PLA), poly(acrylic acid) (PAA), poly-3-hydroxybutyrate (P3HB), poly(hydroxybutyrate-co-hydroxyvalerate), and polyethylene glycol (PEG). In embodiments, a particle includes blends of other polymer materials to modulate a particle's surface properties. For example, the blend may include non-degradable polymers that are used in the art, such as polystyrene. Thus, in embodiments, a degradable polymer or polymers from above are blended to create a copolymer system. In yet other embodiments, the presently disclosed particle comprises a polymer blend of PBAE, e.g., a mixture of PBAE polymers.

In embodiments, the particles are spherical in shape. In embodiments, the particles have a non-spherical shape. In embodiments, the particles have an ellipsoidal shape with an aspect ratio of the long axis to the short axis between 2 and 10.

In certain embodiments, nanoparticles formed through the presently disclosed procedures that encapsulate active agents, such as a protein, are themselves encapsulated into a larger nanoparticle, microparticle, or device. In some embodiments, this larger structure is degradable and in other embodiments it is not degradable and instead serves as a reservoir that can be refilled with the nanoparticles. These larger nanoparticles, microparticles, and/or devices can be constructed with any biomaterials and methods that one skilled in the art would be aware. In some embodiments they can be constructed with multi-component degradable cationic polymers as described herein. In other embodiments, they can be constructed with FDA-approved biomaterials, including, but not limited to, poly(lactic-co-glycolic acid) (PLGA). In the case of PLGA and the double emulsion fabrication process as an example, the nanoparticles are part of the aqueous phase in the primary emulsion. In the final PLGA nano- or microparticles, the nanoparticles will remain in the aqueous phase and in the pores/pockets of the PLGA nano- or microparticles. As the microparticles degrade, the nanoparticles will be released, thereby allowing sustained release of the nanoparticles comprising the active agents. In particular embodiments, the nanoparticle or microparticle of the PBAE of formula (I) or formula (II) is encapsulated in a poly(lactic-co-glycolic acid) (PLGA) nanoparticle or microparticle.

In embodiments, a particle of the presently disclosed subject matter comprises a ligand on its surface which specifically targets the particle to a cell of interest. Thus, such a particle delivers its cargo primarily to a target cell, e.g., a cell in need of gene editing.

In embodiments, the ligand is an antibody or fragment or portion thereof. The antibody or fragment or portion thereof having binding specificity for a receptor or other target on the surface of the cell of interest. As used herein, the term “antibody” includes antibodies and antigen-binding portions thereof. In some embodiments, the ligand is an antibody (e.g., a monoclonal or polyclonal antibody) or an antibody mimetic, such as a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, or as described in U.S. Pat. Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In embodiments, the ligand specifically binds to a tumor-associated antigen or epitope thereof. Tumor-associated antigens include unique tumor antigens expressed exclusively by the tumor from which they are derived, shared tumor antigens expressed in many tumors but not in normal adult tissues, and tissue-specific antigens expressed also by the normal tissue from which the tumor arose. Tumor-associated antigens can be, for example, embryonic antigens, antigens with abnormal post-translational modifications, differentiation antigens, products of mutated oncogenes or tumor suppressors, fusion proteins, or oncoviral proteins. Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas). Use of ligand that binds to a tumor-associated antigen or epitope thereof, allows delivery of a nucleic acid expressing a gene-editing protein to a cancer cell; in the cancer cell, the gene-editing protein may delete or inactivate a gene responsible for the cancer cell's proliferation, for example.

Ligands can be chemically conjugated to a particle using any available process. Functional groups for ligand binding include COOH, NH₂, SH, maleimide, pyridyl disulfide and acrylate. See, e.g., Hermanson, BIOCONJUGATE TECHNIQUES, Academic Press, New York, 1996. Activating functional groups include alkyl and acyl halides, amines, sulfhydryls, aldehydes, unsaturated bonds, hydrazides, isocyanates, isothiocyanates, ketones, azide, alkyne-derivatives, anhydrides, epoxides, carbonates, aminoxy, furan-derivatives and other groups known to activate for chemical bonding. In some embodiments, a ligand can be bound to the particle through the use of a small molecule-coupling reagent. Non-limiting examples of coupling reagents include carbodiimides, maleimides, N-hydroxysuccinimide esters, bischloroethylamines, and functional aldehydes such as glutaraldehyde, anhydrides and the like. In other embodiments, a ligand is coupled to a particle through affinity binding such as a biotin-streptavidin linkage or coupling. For example, streptavidin can be bound to a particle by covalent or non-covalent attachment, and a biotinylated ligand can be synthesized using methods that are well known in the art.

In embodiments, ligands are conjugated to a particle through use of cross-linkers containing n-hydro-succinimido (NHS) esters which react with amines on proteins. Alternatively, the cross-linkers are employed that contain active halogens that react with amine-, sulfhydryl-, or histidine-containing proteins, or cross-linkers containing epoxides that react with amines or sulfhydryl groups, or between maleimide groups and sulfhydryl groups. In embodiments, ligands and protein complexes are conjugated, e.g., functionalized, to the particles using EDC/NHS (1-ethyl-3-(3-dimethylaminopropyl)carbodiimide hydrochloride/N hydroxysuccinimide) chemistry, which conjugates carboxyl groups of protein ligands to PLGA. In some embodiments, ligands can be engineered with site-specific functional groups (example, such as a free cysteine), to provide consistent, site-directed, attachment to particles. Site directed attachment can be to functional groups of the selected polymers, including amines. In these embodiments, functional domains of ligands can be directed toward the environment and away from the particle surface. These embodiments further provide a controlled orientation more suitable for off-the-shelf pharmaceutical products.

The resulting nanoparticles are non-cytotoxic and are biodegradable with a half-life between 1 and 7 h in aqueous conditions. Moreover, freeze-dried nanoparticles are stable for up to two years when stored at room temperature, 4° C., or −20° C.

In some embodiments, the presently disclosed subject matter also includes a method of using and storing the polymers and particles described herein whereby a cryoprotectant (including, but not limited to, a sugar) is added to the polymer and/or particle solution and it is lyophilized and stored as a powder. Such a powder is designed to remain stable and be reconstituted easily with aqueous buffer as one skilled in the art could utilize.

### B. Pharmaceutical Formulations

In some embodiments, the presently disclosed subject matter provides a pharmaceutical formulation comprising the carboxylated, branched poly(beta-amino ester) (PBAE) of formula (I) or formula (II) and at least one protein. In some embodiments, the pharmaceutical formulation further comprises a nanoparticle or microparticle of the PBAE of formula (I) or formula (II). In certain embodiments, the nanoparticle or microparticle of the PBAE of formula (I) or formula (II) is encapsulated in a poly(lactic-co-glycolic acid) (PLGA) nanoparticle or microparticle.

In some embodiments, the particles are complexed with a gene-editing protein. In such embodiments, the presently disclosed subject matter provides biodegradable nanoparticles to direct efficient site-target disruption, mutation, deletion, or repair of a nucleic acid (e.g., a DNA and/or an RNA). Thus, the presently disclosed subject matter provides an efficient gene therapy platform, involving either ex vivo or in vivo gene and/or transcript editing.

### C. Kits

In some embodiments, the presently disclosed subject matter provides a kit comprising the composition comprising a carboxylated, branched poly(beta-amino ester) (PBAE) of formula (I) or formula (II) and at least one protein in a pharmaceutically acceptable carrier. In certain embodiments, the kit further comprises one of more of multiple dosage units of the composition, a pharmaceutically acceptable carrier, a device for administration of the composition, instructions for use, and combinations thereof.

### D. Methods for Delivering One or More Proteins to a Cell

In some embodiments, the presently disclosed subject matter provides a method for delivering a protein to a cell, the method contacting a cell with the composition of formula (I) or formula (II) or a formulation thereof, wherein the composition comprises at least one protein.

In particular embodiments, the protein is delivered to a cytosol of the cell. In certain embodiments, the method mediates endosomal disruption but may alternatively be used to initiate endosomal internalization of proteins to an endosomal or lysosomal space.

### E. Methods for Gene Editing

In some embodiments, the presently disclosed subject matter provides a method for editing a gene comprising contacting a cell with the composition comprising a carboxylated, branched poly(beta-amino ester) (PBAE) of formula (I) or formula (II), and at least one gene-editing protein. In particular embodiments, the gene-editing protein directs site-specific target DNA disruption, mutation, deletion, or repair. In some embodiments, the composition and cell are contacted in vivo. In other embodiments, the composition and cell are contacted ex vivo.

Accordingly, the presently disclosed particles provide for efficient transfection of cells with a gen-editing protein.

The target DNA may be the cause of a disease or disorder, e.g., due to a genetic mutation (including, but not limited to, a single nucleotide polymorphism or SNP).

The cell can be a eukaryotic cell, such as an animal cell or plant cell, including a mammalian cell, such as a human cell. In some embodiments, including for ex vivo protein delivery, the cell is a stem cell or progenitor cell. The cell may be multipotent or pluripotent. In some embodiments, the cell is a stem cell, such as an embryonic stem cell or adult stem cell. In some embodiments, the cell is a hematopoietic stem cell. In some embodiments, including for in vivo protein delivery, the cell (e.g., target cell) is a cancer cell, malignant cell, or diseased cell.

In some embodiments, the particles are delivered directly to an organism, such as mammalian subject, to thereby direct gene editing in vivo. For gene editing in vivo, particles can be formulated for a variety of modes of administration, including systemic and topical or localized administration.

Thus, the pharmaceutical compositions can be formulated for administered to patients by any appropriate routes, including intravenous administration, intra-arterial administration, subcutaneous administration, intradermal administration, intralymphatic administration, and intra-tumoral administration.

In some embodiments, the composition is lyophilized, and reconstituted prior to administration.

In various embodiments, the nanoparticles carry a gene-editing protein. In some embodiments, the nanoparticles comprise a ribonucleoprotein. That is, in some embodiments, nanoparticles comprise a CRISPR protein (e.g., a Cas9 or Cas9-like protein) and a guide RNA (gRNA).

A gene-editing protein creates a nick or a double-strand break in a target DNA molecule, which inactivates a gene or results in expression (from the gene) of an inactive, reduced-activity, or dominant-negative form of a protein. In some embodiments, the gene-editing protein repairs one or more mutations in a gene or deletes a gene segment, which can be guided by a gRNA with the CRISPR/Cas9 system.

In embodiments, the gene-editing protein relates to CRISPR. CRISPR is described, at least in U.S. Pat. Nos. 8,697,359 and 9,637,739, each of which is hereby incorporated by reference in its entirety. In embodiments of the presently disclosed subject matter, a particle as provided herein comprises a CRISPR-associated protein. While various CRISPR/Cas systems have been used extensively for genome editing in cells of various types and species, recombinant and engineered nucleic acid-binding proteins, such as Cas9 and Cas9-like proteins, find use (e.g., in vitro) in the presently disclosed subject matter. The Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and digests foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference).

In some embodiments, different CRISPR proteins (e.g., Cas9 proteins (e.g., Cas9 proteins from various species and modified versions thereof)) may be advantageous to use in the various provided methods in order to capitalize on various characteristics of the different CRISPR proteins (e.g., for different PAM sequence preferences; for no PAM sequence requirement; for increased or decreased binding activity; for an increased or decreased level of cellular toxicity; for increase or decrease efficiency of in vitro RNP formation; for increase or decrease ability for introduction into cells (e.g., living cells, e.g., living primary cells), etc.). CRISPR proteins from various species may require different PAM sequences in the target DNA. Thus, for a particular CRISPR protein of choice, the PAM sequence requirement may be different than the 5′-XGG-3′ sequence described above. In some embodiments, the protein is an xCas protein having an expanded PAM compatibility (e.g., a Cas9 variant that recognizes a broad range of PAM sequences including NG, GAA and GAT), e.g., as described in Hu et al. (2018) “Evolved Cas9 variants with broad PAM compatibility and high DNA specificity” Nature 556: 57-63, incorporated herein by reference in its entirety.

In some embodiments, the presently disclosed subject matter comprises use of other RNA-guided gene-editing nucleases (e.g., Cpf1 and modified versions thereof, Cas13 and modified versions thereof). For example, in some embodiments, use of other RNA-guided nucleases (e.g., Cpf1 and modified versions thereof) provides advantages—e.g., in some embodiments, the characteristics of the different nucleases are appropriate for methods as described herein (e.g., other RNA-guided nucleases have preferences for different PAM sequence preferences; other RNA-guided nucleases operate using single crRNAs other than cr/tracrRNA complexes; other RNA-guided nucleases operate with shorter guide RNAs, etc.) In some embodiments, the presently disclosed subject matter comprises use of a Cpf1 protein, e.g., as described in U.S. Pat. No. 9,790,490, which is incorporated herein by reference in its entirety.

Many Cas9 orthologs from a wide variety of species have been identified and the proteins share only a few identical amino acids. All identified Cas9 orthologs have the same or similar domain architecture comprising a central HNH endonuclease domain and a split RuvC/RNaseH domain. Cas9 proteins share 4 motifs with a conserved architecture. Motifs 1, 2, and 4 are RuvC like motifs and motif 3 is an HNH motif. In some embodiments, a suitable polypeptide (e.g., a Cas9) comprises an amino acid sequence having 4 motifs, each of motifs 1-4 having at least about 75%, at least about 80%, at least about 85%, at least about 90%, at least about 95%, at least about 99% or 100% amino acid sequence identity to the motifs 1-4 of a known Cas9 and/or Csn1 amino acid sequence.

A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013, incorporated herein by reference), and a large number of Cas9 proteins are listed in supplementary FIG. 1 and supplementary table 1 thereof, which are incorporated by reference herein. Additional Cas9 proteins are described in Esvelt et al., Nat Methods. 2013 November; 10(11):1116-21 and Fonfara et al., “Phylogeny of Cas9 determines functional exchangeability of dual-RNA and Cas9 among orthologous type II CRISPR-Cas systems.” Nucleic Acids Res. 42: 2577-90 (2014), each of which is incorporated herein by reference.

Cas9 proteins, and thus modified Cas9 proteins, from a variety of species find use in the presently disclosed subject matter described herein. While the S. pyogenes and S. thermophilus Cas9 molecules are widely used, Cas9 molecules of, derived from, or based on the Cas9 proteins of other species listed herein find use in embodiments of the presently disclosed subject matter. Accordingly, the presently disclosed subject matter provides for the replacement of S. pyogenes and S. thermophilus Cas9 and modified CRISPR (e.g., Cas9) protein molecules with Cas9 and modified CRISPR protein molecules from the other species, e.g.:

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum |
| IMCC1322 | |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp-B510 |
| 92109262 | Nitrobacter hamburgensis XI4 |
| 148255343 | Bradyrhizobium sp-BTAil |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis LI-82 |
| 218767588 | Neisseria meningitidis Z2491 |
| 15602992 | Pasteurella multocida subsp. multocida |
| 319941583 | Sutterella wads worthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

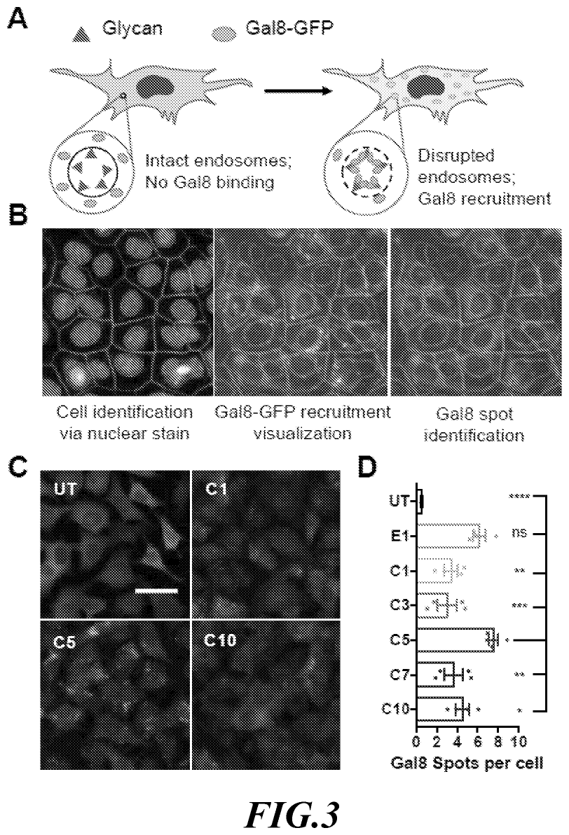

See also U.S. Pat. App. Pub. No. 20170051312 at FIGS. 3, 4, 5, which are incorporated herein by reference.

In some embodiments, the presently disclosed subject matter described herein encompasses the use of a CRISPR protein and/or a CRISPR protein derived from any Cas9 protein (e.g., as listed above) and their corresponding guide RNAs or other guide RNAs that are compatible. The Cas9 from the Streptococcus thermophilus LMD-9 CRISPR1 system has been shown to function in human cells (see, e.g., Cong et al. (2013) Science 339: 819, incorporated herein by reference). Additionally, Jinek showed in vitro that Cas9 orthologs from S. thermophilus and L. innocua, can be guided by a dual S. pyogenes gRNA to cleave target plasmid DNA.

In some embodiments, the presently disclosed subject matter comprises the Cas9 protein from S. pyogenes, e.g., as encoded in a bacterium or codon-optimized for expression in microbial or mammalian cells. For example, in some embodiments, the Cas9 used herein is at least approximately 50% identical to the sequence of S. pyogenes Cas9, e.g., at least 50% identical to the following sequence provided by GenBank Accession Number WP 010922251, incorporated herein by reference (SEQ ID NO: 2):

>Type II CRISPR RNA-guided endonuclease Cas9
[Streptococcus pyogenes]
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKRYTSTKEVLDATLIHQSITGLYETRIDLSQLGGD

In some embodiments, the presently disclosed subject matter comprises use of a nucleotide sequence that is approximately 50%, 55%, 60%, 65%, 70%, 75%, 80%, 85%, 90%, 95%, 99% or 100% identical to a nucleotide sequence that encodes a protein described by SEQ ID NO: 2.

In some embodiments, the Cas9 portion of the CRISPR protein used herein is at least about 50% identical to the sequence of the S. pyogenes Cas9, e.g., at least 50%, 55%, 60%, 65%, 70%, 75%, 80%, 85%, 90%, 95%, 99%, or 100% identical to SEQ ID NO: 2.

In some embodiments, the polypeptide (e.g., the gene-editing nuclease) is a Cas protein, CRISPR protein, or Cas-like protein. “Cas protein” and “CRISPR protein” and “Cas-like protein”, as used herein, includes polypeptides, enzymatic activities, and polypeptides having activities similar to proteins known in the art as, or encoded by genes known in the art as, e.g., Cas1, Cas1B, Cas2, Cas3, Cas4, Cas5, Cas6, Cas7, Cas8, Cas9 (also known as Csn1 and Csx12), Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, homologs thereof, or modified versions thereof, e.g., including any of these Cas proteins, CRISPR proteins, and/or Cas-like proteins known in the art.

In embodiments, the presently disclosed subject matter comprises use of a polypeptide (e.g., a Type V/Type VI protein) such as Cpf1 or C2c1 or C2c2 and homologs and orthologs of a Type V/Type VI protein such as Cpf1 or C2c1 or C2c2 to provide a CRISPR protein. Embodiments encompass Cpf1, modified Cpf1 (e.g., a modified Cpf1), and CRISPR systems related to Cpf1, modified Cpf1, and chimeric Cpf1. In some embodiments, the polypeptide (e.g., a Type V/Type VI protein) such as Cpf1 or C2c1 or C2c2 is from a genus that is, e.g., Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter; Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, or Acidaminococcus. In some embodiments, the polypeptide (e.g., a Type V/Type VI protein) such as Cpf1 or C2c1 or C2c2 is from an organism that is, e.g., S. mutans, S. agalactiae, S. equisimilis, S. sanguinis, S. pneumonia; C. jejuni, C. coli; N salsuginis, N. tergarcus; S. auricularis, S. carnosus; N meningitides, N gonorrhoeae; L. monocytogenes, L. ivanovii; C. botulinum, C. difficile, C. tetani, or C. sordellii. See, e.g., U.S. Pat. No. 9,790,490, incorporated herein by reference in its entirety. In some embodiments, a Cpf1 protein finds use as described in U.S. Pat. App. Pub. No. 20180155716, which is incorporated herein by reference.

In some embodiments, differences from SEQ ID NO: 2 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11):1116-21 and Fonfara et al., Nucl. Acids Res. (2014) 42 (4): 2577-2590, each of which is incorporated herein by reference.

Thus, in some embodiments, the Cas9 polypeptide is a naturally-occurring polypeptide. In some embodiments, the Cas9 polypeptide is not a naturally-occurring polypeptide (e.g., a chimeric polypeptide, a naturally-occurring polypeptide that is modified, e.g., by one or more amino acid substitutions produced by an engineered nucleic acid comprising one or more nucleotide substitutions, deletions, insertions).

In some embodiments, the presently disclosed subject matter relates to a protein that is a CRISPR protein derivative. In some embodiments, the protein is a Type II Cas9 protein. In some embodiments, the Cas9 has been engineered to partially remove the nuclease domain (e.g., a “dead Cas9” or a “Cas9 nickase”; see, e.g., Nature Methods 11: 399-402 (2014), incorporated herein by reference). In some embodiments, the RNP protein is a protein from a CRISPR system other than the S. pyogenes system, e.g., a Type V Cpf1, C2c1, C2c2, C2c3 protein and derivatives thereof

In some embodiments, the polypeptide is a chimeric or fusion polypeptide, e.g., a polypeptide that comprises two or more functional domains. For example, in some embodiments, a chimeric polypeptide interacts with (e.g., binds to) an RNA to form an RNP (described above). The RNA guides the polypeptide to a target sequence within target nucleic acid. Thus, in some embodiments a chimeric polypeptide binds target nucleic acid.

In some embodiments, the presently disclosed subject matter comprises use of an RNA-targeting protein (e.g., Cas13 and/or a modified Cas13), which works according to a similar mechanism as Cas9. In addition to targeting genomic DNA, Cas9 and other CRISPR related proteins (e.g., Cas13) also target RNAs directed by gRNAs (see, e.g., Abudayyeh et al. (2017) “RNA targeting with CRISPR-Cas13” Nature 550: 280, incorporated herein by reference). Thus, in some embodiments, gRNAs complex with Cas9 or other RNA-guided nucleases (e.g., a class 2 type VI RNA-guided RNA-targeting CRISPR-Cas effector (e.g., Cas13), a Cpf1, etc.) to modify (e.g., edit) an RNA (e.g., RNA transcripts and non-coding RNAs). Accordingly, in some embodiments, the presently disclosed subject matter relates to modifying (e.g., editing) a target RNA using guide RNAs in complex with a CRISPR protein (e.g., an RNA-targeting affinity-tagged Cas13).

In some embodiments, the polypeptide comprises a segment comprising an amino acid sequence that is at least approximately 75% amino acid identical to amino acids 7-166 or 731-1003 of any of the amino acid sequences set forth as SEQ ID NOs: 1-256 and 795-1346 of U.S. Pat. App. Pub. No. 20170051312, incorporated herein by reference.

When Cas9 is associated with its gRNA (or components thereof), e.g., to form a ribonucleoprotein (RNP), it is able to modify a specific region of a nucleic acid (e.g., a DNA and/or an RNA) by single-strand nicking, double-strand break, and/or DNA binding.

Accordingly, in some embodiments, the presently disclosed subject matter comprises use of a ribonucleoprotein (RNP) comprising a CRISPR protein. In some embodiments, the presently disclosed subject matter comprises use of a RNP complex comprising a Cas9 or Cas9-like protein and one or more RNA molecules (e.g., a gRNA (e.g., a nucleic acid-targeting RNA, an activator-RNA and a targeter-RNA, a crRNA and a tracrRNA; a dgRNA; a sgRNA)). In some embodiments, the presently disclosed subject matter comprises use of a ribonucleoprotein (RNP) complex comprising a Cas9 or Cas9-like protein as described herein and one or more RNA molecules (e.g., a gRNA (e.g., a nucleic acid-targeting RNA, an activator-RNA and a targeter-RNA, a crRNA and a tracrRNA; a dgRNA; a sgRNA)).

In some embodiments, the presently disclosed subject matter comprises use of a plurality of RNPs, e.g., to produce multiple double-stranded breaks in a nucleic acid. For instance, in some embodiments the presently disclosed subject matter comprises use of a first RNP comprising a CRISPR protein (e.g., Cas9 or Cas9-like protein) and a first RNA molecule or first set of RNA molecules (e.g., a gRNA (e.g., a nucleic acid-targeting RNA, an activator-RNA and a targeter-RNA, a crRNA and a tracrRNA; a dgRNA; a sgRNA)) and a second RNP comprising a CRISPR protein (e.g., a Cas9 or Cas9-like protein) and a second RNA molecule or second set of RNA molecules (e.g., a gRNA (e.g., a nucleic acid-targeting RNA, an activator-RNA and a targeter-RNA, a crRNA and a tracrRNA; a dgRNA; a sgRNA)).

The RNA provides target specificity to the RNP complex by comprising a nucleotide sequence that is complementary to a target sequence of a target nucleic acid. The polypeptide of the complex (e.g., a CRISPR protein) provides binding and nuclease activity. In other words, the polypeptide is guided to a nucleic acid sequence (e.g., a DNA sequence (e.g., a chromosomal sequence, an extrachromosomal sequence (e.g., an episomal sequence, a minicircle sequence, a mitochondrial sequence, a chloroplast sequence, etc.), a cDNA sequence) or an RNA sequence (e.g., a transcript sequence, a functional RNA sequence)) by virtue of its association with at least the protein-binding segment of the nucleic acid-targeting RNA.

In embodiments a gene-editing protein comprises a nuclear-localization sequence or a mitochondrial-localization sequence.

In some embodiments non-peptide biological small molecules or biomacromolecules are encapsulated by the polymeric nanoparticles including sugars, polysaccharides, carbohydrates, morpholinos, and/or nucleic acids.

### Example 1 Carboxylated Branched Poly(Beta-Amino Ester) Nanoparticles Enable Robust Cytosolic Protein Delivery and Crispr/Cas9 Gene Editing

### 1.1 Overview

Efficient cytosolic protein delivery is necessary to fully realize the potential of protein therapeutics. Current methods of protein delivery often suffer from low serum tolerance and limited in vivo efficacy. The presently disclosed subject matter provides the synthesis and validation of a previously unreported class of carboxylated branched poly(beta-amino ester)s (PBAEs), which can self-assemble into nanoparticles for efficient intracellular delivery of a variety of different proteins. In vitro, nanoparticles enabled rapid cellular uptake, efficient endosomal escape, and functional cytosolic protein release into cells in media containing 10% serum. Moreover, nanoparticles encapsulating CRISPR/Cas9 ribonucleoproteins (RNPs) induced robust levels of gene knock-in (4%) and gene knock-out (>75%) in several cell types. A single intracranial administration of nanoparticles delivering a low RNP dose (3.5 pmol) induced robust gene editing in mice bearing engineered orthotopic murine glioma tumors. The presently disclosed self-assembled polymeric nanocarrier system enables a versatile protein delivery and gene editing platform for biological research and therapeutic applications.

### 1.2 Background

Since the introduction of the first recombinant protein drug— human insulin—in 1982, Clark et al., 1982, the number of therapeutic proteins and the frequency of their use have markedly increased. These diverse and dynamic macromolecules have been used to treat diseases ranging from metabolic disorders to cancer, Leader et al., 2008, and are important in applications such as genome editing, Paschon et al., 2019, and synthetic biology. Lienert et al., 2014. Their high molecular weight and overall hydrophilicity, however, render most proteins essentially membrane impermeable, Postupalenko et al., 2015, limiting most current protein therapeutics to extracellular targets. As proteins have the potential to target intracellular pathways with high specificity and fewer side effects, Southwell et al., 2008, it is imperative to develop novel strategies for efficient, functional, and cytosolic protein delivery.

Cytosolic protein delivery vehicles must overcome several barriers, such as cargo encapsulation, cellular internalization, escape from endo/lysosomes, and cytosolic cargo release. Rui et al., 2019a. One well-characterized approach is the covalent modification of the protein of interest with protein transduction domains (PTDs), such as the TAT protein from human immunodeficiency virus. Schwarze et al., 1999. This strategy has been shown to enable rapid cellular internalization of a wide variety of proteins but requires chemical modifications that could alter the bioactivity of the native protein. More recently, several studies have reported the use of self-assembled protein delivery vehicles based on lipid-like, Wang et al., 2016, polymeric, Zhang et al., 2018; Chang et al., 2017, or hybrid materials. Cheng et al., 2018; Chen et al., 2018; and Alsaiari et al., 2017. These methods still face limitations, such as the need for purification steps, low protein loading efficiency, and limited applicability to certain cargo types, prompting the need for improved self-assembled protein delivery systems.

Hyperbranched cationic poly(β-amino ester)s (PBAEs) have recently generated interest as an efficient gene delivery material for highly negatively-charged nucleic acids. Gao et al., 2016; Wilson et al., 2019; Rui et al, 2019b. These amphiphilic, pH-sensitive polymers are synthesized via facile Michael addition reactions and have been shown to have robust transfection capabilities under challenging conditions, as well as efficient endosomal escape properties. Cationic polymers, such as PBAEs, however, form self-assembled nucleic acid nanoparticles mainly through electrostatic interactions, which are generally insufficient to encapsulate proteins of diverse surface charge.

### 1.3 Scope of Work

The presently disclosed subject matter provides the synthesis and validation of a new class of hyperbranched PBAE biomaterials containing both cationic and anionic charges. This characteristic was accomplished through polymer end-capping with carboxylate ligands derived from amino acid-like precursors. Polymers were assembled into nanoparticles with proteins by simple mixing in aqueous buffer. Without wishing to be bound to any one particular theory, it is thought that the carboxylate ligands can enhance polymer-protein interactions for nanoparticle assembly via increased hydrogen bonding and hydrophobic effects in addition to electrostatic interactions. Furthermore, it was found that differential polymer end-group hydrophobicity affected protein complexation capabilities, as well as nanoparticle internalization and endosomal escape. The presently disclosed delivery platform enabled functional cytosolic delivery of proteins ranging from 27 kD to 160 kD in molecular weight with varying surface charges. Encapsulation of Cas9 ribonucleoproteins (RNPs) enabled efficient gene editing in vitro and in vivo, further highlighting the robustness and therapeutic utility of the presently disclosed nanocarriers.

### 1.4 Results

### 1.4.1 Polymer Synthesis and Screening

Hyperbranched PBAEs were synthesized via a step-wise copolymerization reaction between acrylate-containing monomers bisphenol A glycerolate (1 glycerol/phenol) diacrylate (B7) and trimethylolpropane triacrylate (B8), and amino alcohol monomer 4-amino-1-butanol (S4). B monomers were added in molar excess to yield acrylate-terminated polymers, which were end-capped with the diamine-containing small molecule 1,3-diaminopropane (E1) to yield E1 base polymers (FIG. 1A). These polymers underwent a second round of end-capping reactions with carboxylate ligands (FIG. 7), which were synthesized via reaction of a series of amino acid-like precursors with acryloyl chloride. Ligands were named according to the number of carbon atoms between the amide and carboxylic acid groups, with C1 deriving from the amino acid glycine (FIG. 8). Five carboxylate ligands ranging from C1 to C10 were synthesized to investigate the effect of end-cap hydrophobicity on the protein encapsulation and delivery capabilities of the polymers. Without wishing to be bound to any one particular theory, that the combination of the biodegradable and hyperbranched polymer backbone, which has been shown to be amphiphilic and pH sensitive, Wilson et al., 2019, and carboxylate end-capping ligands, capable of forming hydrogen bonds and salt bridges with proteins, would result in a versatile protein delivery polymer platform.

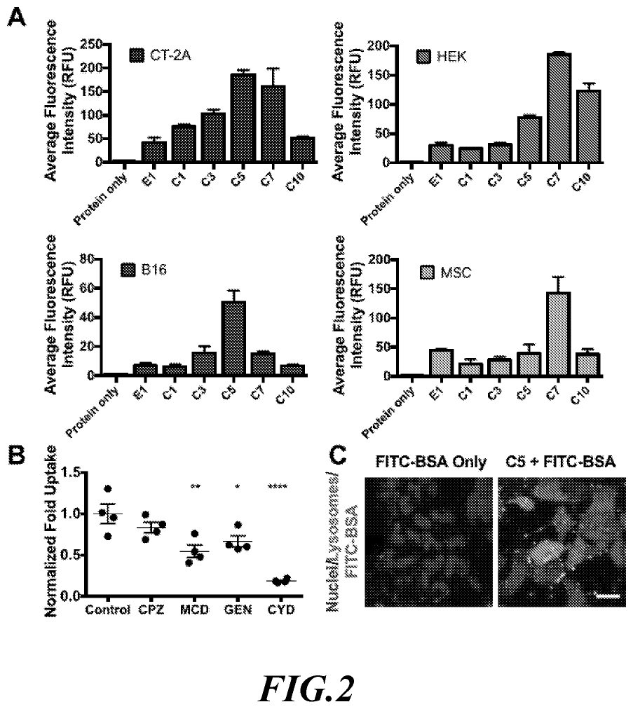

To investigate the protein encapsulation capabilities of the polymers, self-assembled polymeric nanoparticles were formulated with bovine serum albumin (BSA). At a polymer-protein weight ratio (wt/wt) of 30, all carboxylate terminated polymers in the series formed nanoparticles ranging about 200 nm to about 500 nm in hydrodynamic diameter with surface charges close to neutral (FIG. 1C), whereas the E1-terminated polymer, useful for self-assembly with nucleic acids, Shmueli et al., 2012, failed to effectively form nanoparticles with BSA. The diameter of the nanoparticles formulated from carboxylated polymers had a biphasic response dependent on the number of carbon atoms between the amide and carboxylic acid groups. Polymers end-capped with ligands C5 and C7 formed the smallest nanoparticles, and polymers with lower or higher end-cap carbon length formed much larger nanoparticles. Moreover, the same biphasic response was observed functionally when these polymers were used to deliver fluorescein isothiocyanate (FITC)-labeled BSA intracellularly (FIG. 2A). In all four cell lines evaluated (CT-2A murine glioma, human embryonic kidney (HEK)-293T, B16-F10 murine melanoma, and MSC-083 primary human adipose-derived mesenchymal stem cells), polymers surface engineered with C5 or C7 moieties enabled the highest levels of intracellular nanoparticle-mediated protein uptake. These data indicate that end-cap hydrophobicity and the spacing length between charges play a major role in the interactions between polymer and protein during nanoparticle self-assembly, which, in turn, affects interactions between nanoparticles and cells during cellular uptake. The high levels of protein nanoparticle uptake did not result in notable levels of cytotoxicity and the viability of cells treated with nanoparticles was >70% for all polymers and cell lines tested when polymers were used at standard conditions <0.15 mg/mL (FIG. 9).

When nanoparticle internalization pathways were probed by selectively inhibiting endocytosis pathways using small-molecule drugs, it was found that pre-treatment with cytochalasin-D decreased nanoparticle uptake by over 80%, suggesting that nanoparticles were internalized primarily by macropinocytosis (FIG. 2B). Methyl-β-cyclodextrin and genistein also significantly decreased cellular uptake while chlorpromazine had negligible effects, indicating that nanoparticles also were taken up through lipid raft- and caveolin-mediated endocytosis, but not through clathrin-mediated endocytosis. Finally, confocal laser scanning microscopy images of cells after 4 h incubation with C5/FITC-BSA nanoparticles revealed diffuse FITC-BSA signal throughout the cytosol, indicating that nanoparticles successfully escaped degradative endo-lysosomes to enable cytosolic protein delivery (FIG. 2C and FIG. 10).

### 1.4.2 Endosomal Disruption Characterization Via Gal8-GFP Recruitment Assay

The endosomal escape capabilities of carboxylated branched PBAE nanoparticles was further characterized using an assay based on the recruitment of galectin 8 (Gal8) to disrupt endosomal membranes similar to the method recently innovated by Kilchrist et al., 2019. Gal8 is a cytosolic protein that binds to glycosylation moieties located selectively on the inner leaflets of endosomal membranes. Using a PiggyBac transposon, a cell line stably expressing a Gal8-green fluorescent protein (GFP) fusion protein was created. Endosomal rupture exposes Gal8 binding sites to cytosolic Gal8-GFP, and Gal8-GFP recruitment results in punctate fluorescent spots at disrupted endosomes (FIG. 3A). After staining with Hoechst 33342 nuclear dye to allow for cell identification, automated high content imaging analysis can then be used to identify punctate Gal8-GFP spots and calculate the number of Gal8-GFP spots per cell as an indicator of the level of nanoparticle-mediated endosomal disruption (FIG. 3B).

The presently disclosed results revealed that among the carboxylate end-capped polymers, polymer C5 enabled the highest level of endosomal disruption (FIG. 3D). This observation was not due to the buffering capabilities of these polymers, as pH titration experiments showed that there was no significant difference in buffering capacity among the different carboxylated polymers (FIG. 11A). It is also important to note that there was no significant difference between the Gal8-GFP recruitment levels of nanoparticles formed with the E1 base polymer and those formed with polymer C5. Polymer end-capping with carboxylate ligands of shorter chain lengths (e.g., C1 and C3) resulted in a decrease in endosomal disruption levels. This may be explained by the fact that the E1 monomer itself interacts with endosomal membranes in a way that causes disruption, as was demonstrated in previous reports using this molecule as an end cap to efficiently deliver plasmid DNA. Shmueli et al., 2012. Further end-capping with carboxylate ligands masked this effect, and endosomal disruption became dependent on hydrophobic chain length. Certain PBAEs have also been shown to form polymer-only, micellar nanoparticles in the absence of nucleic acid or protein cargo due to their amphiphilic structure. Wilson et al., 2017. Thus, the paradoxical low FITC-BSA uptake and high Gal8-GFP recruitment observed in E1 nanoparticles could be explained by E1 base polymers not adequately forming protein-encapsulated nanoparticles and mainly forming polymer-only nanoparticles, which caused endosomal disruption after endocytosis. Together, our data indicate that polymer C5 outperformed all other polymers in the series and was chosen for use in all subsequent experiments.

### 1.4.3 Robustness of C5-PBAE Nanoparticles

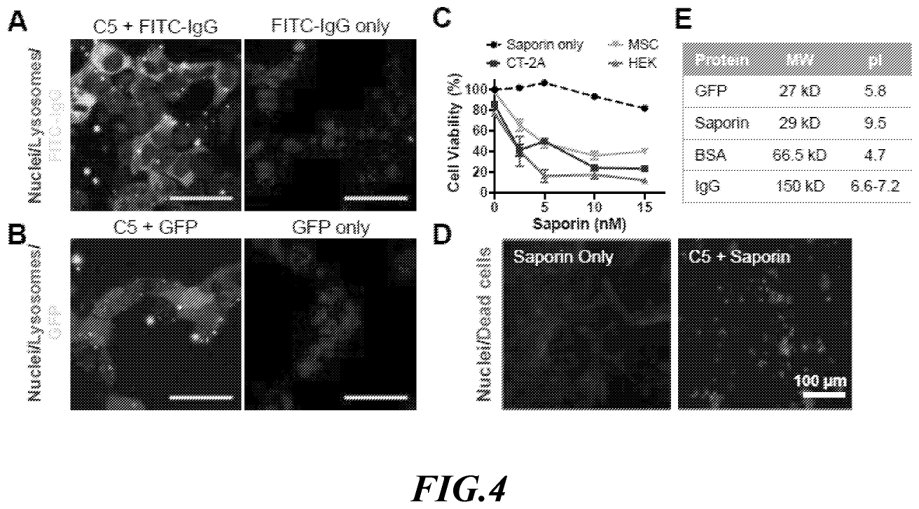

The robustness of C5 end-capped polymers were further examined by utilizing them for cytosolic delivery of a variety of proteins. C5 polymers successfully encapsulated FITC-labeled human IgG (FITC-IgG) and GFP, respectively, and enabled diffuse cytosolic delivery of both (FIG. 4). To investigate the capability for functional protein delivery, C5 polymers were further utilized to encapsulate the ribosome-inactivating protein saporin, a potent toxin lacking cellular internalization domains, Lombardi et al., 2010, (FIG. 4C). In all three cell lines tested, C5/saporin nanoparticles induced high levels of cell death even at very low saporin doses (EC₅₀<5 nM). This observation indicates that C5 nanoparticles enabled functionally intact saporin proteins to reach the ribosome, their intracellular sites of action, with high efficiency. In contrast, unencapsulated saporin could not be internalized on its own and resulted in negligible cytotoxicity even at high concentrations. The presently disclosed data demonstrate that C5 end-capped branched PBAEs are a versatile and robust protein delivery platform, enabling cytosolic, functional protein delivery to a variety of cell lines. More importantly, the polymers are largely agnostic to the size and surface charge of the protein cargo that they carry (FIG. 4E & Table 1), unlike traditional PBAEs that depend on electrostatic interactions and can only encapsulate strongly negatively charged cargos, such as nucleic acids.

TABLE 1
Characteristics of proteins and encapsulated C5 nanoparticles
and optimal nanoparticle formulations used in this study.

| | | | Nanoparticle Characteristics | | | | | |
|---|---|---|---|---|---|---|---|---|
| | | | | | | | Optimal | |
| | Protein | | | | Optimal | | Polymer | |
| | Characteristics | | Size | Zeta | Protein | | Dose | Equivalent |
| Protein | MW | pl | (nm) | (mV) | Dose | | (mg/mL) | w/w |
| GFP | 27 kD | 5.8 | 150 ± 50 | 9.9 ± 0.7 | 300 | ng | 0.075 | 30 |
| Saporin | 29 kD | 9.5 | 120 ± 30 | 8.7 ± 0.4 | 2.5-15 | nM | 0.075 | 2600-175 |
| BSA | 66.5 kD | 4.7 | 160 ± 60 | 5 ± 1 | 300 | ng | 0.075 | 30 |
| IgG | 150 kD | 6.6-7.2 | 120 ± 20 | −1 ± 1 | 300 | ng | 0.075 | 30 |
| Cas9 | 163 kD | 9 | 180 ± 10 | 12.3 ± 0.2 | 690 | ng | 0.1 | 22 |

### 1.4.4 CRISPR Gene Editing Through RNP Delivery In Vitro

C5 polymers also were used to encapsulate and deliver Cas9 ribonucleoproteins (RNPs) to enable CRISPR gene editing in vitro. In these experiments, Cas9 protein and gene-targeting short guide RNA (sgRNA) were first incubated together at room temperature for 10 minutes to allow RNP self-assembly, then simply mixed with polymers to form nanoparticles. Delivery of RNPs targeting the GFP gene in cells constitutively expressing the GFP reporter resulted in 77% GFP knockout in HEK cells and 47% GFP knockout in GL261 murine glioma cells, as quantified by flow cytometry (FIG. 5B). Surveyor mutation detection assay also was performed to verify that loss in GFP fluorescence was due to perturbation in genomic DNA (FIG. 5C). RNPs were membrane impermeable on their own and treatment with RNP alone yielded negligible levels of gene editing.

Next, the capability of C5/RNP nanoparticles to edit an endogenous gene through homology directed repair (HDR) was investigated. Cas9 protein was first self-assembled into RNPs with sgRNA targeting the human CXCR4 gene, and RNPs were further mixed with a single-stranded DNA (ssDNA) repair template before mixing with C5 polymer. The ssDNA repair template included approximately 80 nucleotide (nt) homology arms flanking a 12 nt insert containing a Hind III restriction enzyme site (FIG. 5D). Successful HDR was quantified by Hind III restriction digest of PCR amplicons of the genomic CXCR4 site while total amount of editing (nonhomologous end joining (NHEJ) and HDR) was quantified using the Surveyor® mutation detection assay. The presently disclosed results indicate that C5 nanoparticles successfully delivered the combination of RNP+ssDNA into HEK-293T cells. Gel electrophoresis analysis of cleavage products indicate that 4% HDR was achieved while over 50% total editing was achieved (FIG. 5E and FIG. 5F). Inclusion of a ssDNA template into the nanoparticle self-assembly process did not change the total level of editing achieved. Inference of CRISPR Edits (ICE), Hsiau et al., 2018, analysis of Sanger sequencing results confirmed the presence of a 12 bp insert in 2.5% of DNA sequences (FIG. 5G).

### 1.4.5 Validation of CRISPR-Stop Reporter System to Assess Gene Deletion

A CRISPR-stop reporter construct was engineered wherein a 630-bp stop-of-transcription cassette is placed upstream of a red-enhanced nanolantern (ReNL) fluorescent reporter (FIG. 6A). This CRISPR-stop construct was integrated into the genomic DNA of GL261 and B16-F10 cells via a PiggyBac transposon, and targeting CRISPR RNPs to regions flanking the stop cassette resulted in deletion of the stop cassette and turning on of ReNL fluorescence. This system was chosen to evaluate in vivo gene editing as gain-of-function ReNL expression via dual-cut gene deletion could be easily and clearly detected.

In vitro assessment of this CRISPR-stop system using C5/RNP nanoparticles indicated that 16% and 43% editing were achieved in GL261 and B16 cells, respectively (FIG. 12A). Compared to commercially available CRISPR delivery agents, C5/RNP nanoparticles enabled significantly higher editing levels than Lipofectamine CRISPRMax at all RNP doses tested and significantly higher editing levels than jetCRISPR at equimolar RNP doses tested (FIG. 12B). jetCRISPR enabled significantly higher levels of editing than C5/RNP nanoparticles only when twice the RNP dose was used, further demonstrating the utility of the C5/RNP nanoparticle system in delivering CRISPR RNPs.

This reporter system also allowed the stability of the presently disclosed nanoparticles to be easily assessed under physiological conditions. C5/RNP nanoparticles were preincubated in serum-containing complete cell culture media at 37° C. for up to 4 hours before adding to cells and assessed their ability to induce gain-of-function CRISPR-stop edits (FIG. 13A). Flow cytometry data revealed that no significant loss of nanoparticle efficacy was observed until preincubation time reached 4 hours, at which time delivery efficacy dropped by 25%. This observation is likely due to PBAE hydrolysis and is consistent with previous reports of PBAE half-life in aqueous conditions of 4 to 6 hours, a benefit to facilitate fast biodegradation and minimized toxicity in vivo. Sunshine et al., 2012. To achieve greater long-term stability of nanoparticles, as would be required for storage and supply chain management, we demonstrated that C5/RNP nanoparticles retain their efficacy following lyophilization with sucrose as a cryoprotectant, which may be the first documented case of a functional lyophilized RNP formulation (FIG. 13B). Guerrero-Cázares et al., 2014.

### 1.4.6 CRISPR Editing in Murine Glioma Tumors In Vivo

Lastly, the ability of C5/RNP nanoparticles to enable CRISPR gene editing in vivo was investigated. In vivo assessment of gene editing was performed following intracranial implantation of GL261 cells constitutively expressing the CRISPR-stop construct into C57BL/6J mice. C5/RNP nanoparticles were infused intracranially through convection-enhanced delivery (CED) 10 days after tumor inoculation, and mice were euthanized and brains were extracted 6 days after nanoparticle CED. Histological analysis of mouse brains treated with C5/RNP nanoparticles (3.5-pmol RNP dose with 15 w/w polymer) revealed bright ReNL fluorescence within the tumor bulk, which was not observed in mice that received naked RNP infusion only (FIG. 6B and FIG. 15). Although the brightest ReNL signal was localized in closest proximity to the injection site, ReNL expression could be detected several millimeters away from the primary injection site. These results demonstrate proof of principle that C5/RNP nanoparticles can enable localized CRISPR gene editing in vivo.

### 1.5 Discussion

Functional cytosolic protein delivery holds great value in biological research, as well as therapeutic applications by enabling the perturbation of intracellular pathways previously undruggable by small-molecule drugs. To overcome the intrinsic cell membrane impermeability of many proteins, a series of carboxylated, branched PBAEs for the encapsulation of a variety of different protein types into self-assembled nanoparticles was synthesized and validated. Carboxylate ligand chain length and hydrophobicity played an important role in polymer-protein interactions, as well as the ability for protein-encapsulated nanoparticles to interact with cellular and endosomal membranes. Polymers terminated with C5, a carboxylate ligand of moderate hydrophobicity, outperformed other ligands in the series both in the level of cellular internalization, as well as endosomal disruption. The superior performance of C₅ over end-caps of lesser hydrophobicity could be explained by the fact that increased hydrophobicity facilitates nanoparticle stabilization through hydrophobic effects. Furthermore, the hydrocarbon chains in the polymer end-group could also interact with membranes, facilitating cellular internalization as well as endosomal escape through transient membrane perturbations. A similar phenomenon has been extensively reported with lipid-like materials and also might be applicable here. Rehman et al., 2013. On the other hand, polymer end-groups, such as C10, may be too hydrophobic, or else allow too long of a linker length, to efficiently interact with proteins. For example, a potential collapse of the hydrocarbon tail in aqueous buffer could obstruct interactions between the carboxylic acid functional group with proteins and cell membranes. This biphasic response is consistent with that reported by Ayala et al when similar amino acid analogs were utilized for hydrogel synthesis. Ayala et al., 2011.

The robustness of the presently disclosed nanoparticle system was further demonstrated by cytosolically delivering a variety of proteins of different size and surface charge. The ability to functionally deliver the ribosome-inactivating protein saporin, which has an isoelectric point of 9.5, Lombardi et al., 2010, and is thus strongly cationic at the pH of nanoparticle formation, validates the hypothesis that carboxylated PBAEs can rely on interactions beyond purely electrostatic forces to complex protein cargo into nanoparticles. This result is a significant innovation upon traditional gene delivery PBAEs engineered to complex only highly anionic nucleic acid cargos through charge interactions. Rui et al., 2017.

Finally, it was demonstrated that C5 polymers were capable of functional delivery of Cas9 RNPs to enable CRISPR gene editing. In vitro delivery of RNPs targeting a GFP reporter gene resulted in nearly 80% GFP knockout following NHEJ. This level of gene knockout is comparable to that achieved by the DNA nanoclew system developed by Sun et al., 2015, and significantly higher than that reported by Alsaiari et al., 2017, using ZIF-8 metal-organic framework nanoparticles for CRISPR RNP delivery. Compared to commercially available CRISPR delivery reagents Lipofectamine CRISPRMax and jetCRISPR, C5/RNP nanoparticles enabled significantly higher levels of gene editing at manufacturer-recommended RNP doses. Further, co-delivery of RNPs targeting the human CXCR4 gene and a ssDNA repair template in the same self-assembled nanoparticle enabled 4% HDR in HEK-293T cells, which is significantly higher than that achieved by the CRISPR-Gold system developed by Lee et al., 2017, in the same cell line when scaled by the RNP dose delivered. For translation considerations, the C5/RNP+ssDNA encapsulated nanoparticles can be formulated by simple mixing with polymers while the aforementioned CRISPR-Gold requires a multistep synthesis scheme including covalent conjugation of DNA sequences.

C5/RNP nanoparticles induced CRISPR gene editing in vivo, as well using a challenging reporter model requiring a 631 bp deletion for gain-of-function fluorescence. In a proof-of-principle study, it was demonstrated that deletion of an expression stop cassette resulted in gain of function ReNL reporter fluorescence upon intracranial injection of C5/RNP nanoparticles in a mouse glioma model. The highest levels of editing occurred near the primary nanoparticle infusion site, covering a region in the brain approximately 0.4 mm² in area, which is comparable to that reported by Wang et al., 2016, who used bio-reducible lipids to deliver 5 pmol supercharged GFP-Cre to mouse brains containing a CRISPR-stop reporter system in which the stop cassette is flanked by LoxP sites. In another study, Staahl et al., 2017, utilized a protein engineering approach to enable cellular internalization by adding 4×nuclear localization signal (NLS) residues to the N-terminus of the Cas9 protein but required an order of magnitude higher RNP dose to achieve wide-spread editing. Intracranial injection of 4 pmol modified RNPs enabled gain-of-function tdTomato fluorescence in mouse brain regions similar in area to that observed by Wang et al., 2016. In comparing in vivo editing efficiency, it is important to note that gene editing occurred in primary mouse neurons in the two abovementioned studies while the presently disclosed study investigated gene editing in orthotopic mouse brain tumors. The bright ReNL signal induced by the C5/RNP nanoparticles, however, highlight their robust intracellular delivery capabilities.

A putative advantage of the presently disclosed polymeric nanoparticle-based protein delivery system is its potential ability to evade immune responses. It has been demonstrated that PBAE nanoparticles optimized for nucleic acid delivery could be administered repeatedly to immunocompetent animals without a reduction in transfection efficacy, Patel et al., 2019, indicating that neutralizing antibodies were not formed against the nanoparticles. Without wishing to be bound to any one particular theory, it is thought that the presently disclosed nonviral protein delivery system could have similarly low levels of vector-mediated immune responses, which may be a significant advantage over traditional viral delivery vectors for which immunogenicity is a serious concern. Immunogenicity to Cas9 protein cargo may be a concern for direct in vivo CRISPR editing in human patients as Charlesworth et al., 2019, recently reported that preexisting immunity against spCas9 is likely to limit the editing efficacy of CRISPR RNPs delivered to human patients. Polymeric nanoparticle encapsulation may attenuate immune responses against the protein cargo itself by protecting against circulating neutralizing antibodies, enabling CRISPR gene editing in patients with preexisting immunity. This effect was not evaluated in the current study but would be an interesting direction for future investigation.

### 1.6 Summary

In summary, provided herein is a polymeric nanoparticle system that can encapsulate and enable robust cytosolic delivery of a variety of different protein types, including potent cytotoxic agents, as well as CRISPR/Cas9 RNPs. RNP delivery in vitro and in vivo induced high levels of gene editing at relatively low RNP doses. Biodegradable nanoparticles were formulated via a facile, highly scalable self-assembly process that also is amenable to lyophilization and storage. This versatile protein delivery platform provides a powerful tool for biological research, as well as potential therapeutic applications for neurological disorders and beyond.

### 1.7 Materials and Methods

### 1.7.1 Materials

Acryloyl chloride (CAS 814686), glycine (CAS 56-40-6), 4-aminobutanoic acid (CAS 56-12-2), 6-aminocaproic acid (CAS 60-32-2), 8-aminooctanoic acid (CAS 1002-57-9), 11-aminoundecanoic acid (CAS 2432-99-7), bisphenol A glycerolate (1 glycerol/phenol) diacrylate (B7; CAS 4687949), trimethylolpropane triacrylate (B8; CAS 15625895), 1,3-diaminopropane (E1; CAS 109-76-2), FITC-BSA, saporin from S. officinalis seeds, FITC-IgG from human serum, and Cas9-NLS were purchased from Sigma-Aldrich (St. Louis, Mo.). 4-Amino-1-butanol (S4; CAS 133251005) was purchased from Alfa Aesar (Tewksbury, MA).

### 1.7.2 Carboxylate Ligand Synthesis

Carboxylate ligands were synthesized according to the method by Ayala et al., 2011. Briefly, 0.1 mol carboxylate precursor molecule (listed in FIG. 7B) was added at a 1:1.1 molar ratio with NaOH and dissolved in 80 mL DI water with vigorous stirring in an ice bath. 0.11 mol acryloyl chloride in 15 mL THF was added drop-wise, and the pH of the reaction was maintained at 7.5-7.8 with 1M NaOH solution. The reaction was allowed to proceed overnight before being acidified to the pH listed in FIG. 7B with 1M HCl solution and extracted 3 times with ethyl acetate. The organic layer was collected, dried with sodium sulfate, and the solvent was removed with rotary evaporation to yield a white powder.

### 1.7.3 Polymer Synthesis

Monomers B7 and B8 were dissolved in anhydrous DMSO at 0.8:0.2 molar ratio, and monomer S4 was added at an overall vinyl: amine ratio of 2.2:1 to a final monomer concentration of 150 mg/mL. The reaction was allowed to proceed at 90° C. with stirring overnight, at which point polymers were end-capped by reacting with monomer E1 (0.2 M final concentration in DMSO) at room temperature for 2 hr. The resulting E1 polymers were purified by 2 diethyl ether washes, after which polymers were dissolved at 200 mg/mL in DMSO and end-capped with carboxylate ligands (0.2 M final concentration in DMSO) at room temperature for 2 hr. The resulting carboxylated polymers were further purified by ether precipitation and remaining solvent was removed in a vacuum chamber. Polymers were dissolved in DMSO at 100 mg/mL and stored in single-use aliquots at −20° C. with desiccant.

### 1.7.4 Polymer Characterization: NMR, GPC, and pH Titration

Polymer structure was characterized by nuclear magnetic resonance spectroscopy (NMR) via ¹H NMR in CDCl₃ (Bruker 500 MHz) and analyzed using TopSpin 3.5 software. Polymer molecular weight was characterized by gel permeation chromatography (GPC); polymers were dissolved in BHT-stabilized THF with 5% DMSO and 1% piperidine, filtered through a 0.2-μm PTFE filter, and characterized using GPC against linear polystyrene standards (Waters, Milford, Mass.). pH titrations were performed using a SevenEasy pH meter (Mettler Toledo) with 10 mg of polymer dissolved in 10 mL of 100 mM NaCl acidified with HCl as previously described. Wilson et al., 2019. Polymer was titrated from pH 3.0 to pH 11.0 using 100 mM NaOH added stepwise, and pH was recorded after each addition.

### 1.7.5 Nanoparticle Characterization

Nanoparticles were prepared by dissolving polymer and protein separately in 25 mM sodium acetate (NaAc, pH 5), mixing the two solutions at a 1:1 volume ratio, and allowing for nanoparticle self-assembly at room temperature for 10 minutes. To prepare nanoparticles encapsulating CRISPR ribonucleoproteins (RNPs), sgRNA and Cas9 protein were first mixed together at a 2:1 molar ratio to allow RNP assembly at room temperature for 10 minutes; RNPs were then mixed with polymers at a 1:1 volume ratio. Nanoparticles were diluted 1:5 in 150 mM PBS to determine particle size and zeta potential in neutral, isotonic buffer. Hydrodynamic diameter was measured via dynamic light scattering (DLS) on a Malvern Zetasizer Pro (Malvern Panalytical); zeta potential was measured via electrophoretic light scattering on the same instrument. Transmission electron microscopy (TEM) images were acquired with a Philips CM120 (Philips Research). Nanoparticles encapsulating BSA (30 w/w) were prepared at a polymer concentration of 1.8 mg/mL in 25 mM NaAc. 30 μL nanoparticles were added to 400-square mesh carbon coated TEM grids and allowed to adhere for 20 minutes. Grids were then rinsed with ultrapure water and allowed to fully dry before imaging.

1.7.6 sgRNA In Vitro Transcription

In vitro transcription was performed using a MEGAshortscript T7 Transcription kit (Invitrogen) and purified using a MEGAclear Transcription Clean-up kit (Invitrogen) following manufacturer's instructions. The DNA templates used for in vitro transcription were synthesized as gBlocks from IDT (sequences listed in Table 2).

TABLE 2
Representative DNA Sequences

| | | | SEQ ID | |
|---|---|---|---|---|
| | | Sequences | NO: | Notes |
| Target | GFP | GGAGCGCACCATCTTCTTCAAGG | 1 | PAM; Postive |
| Sequences | | | | Strand |
| | CRISPER-stop | GTATAGCATACATTATACGAGG | 3 | PAM; |
| | | | | Negative |
| | | | | Strand |
| | CXCR4 | GAAGCGTGATGACAAAGAGGAGG | 4 | PAM; |
| | | | | Negative |
| | | | | Strand |
| sgRNA IVT | GFP | GTTTTTAATACGACTCACTATAGGAGC | 5 | T7 Promoter |
| Template | | GCACCATCTTCTTCAGTTTTAGAGCTAG | | Target |
| | | AAATAGCAAGTTAAAATAAGGCTAGTC | | sequence |
| | | CGTTATCAACTTGAAAAAGTGGCACCG | | gRNAscffold |
| | | AGTCGGTGCTTTTTT | | |
| | CRISPER-stop | GTTTTTAATACGACTCACTATAGGTATA | 6 | |
| | | GCATACATTATACGGTTTTAGAGCTAG | | |
| | | AAATAGCAAGTTAAAATAAGGCTAGTC | | |
| | | CGTTATCAACTTGAAAAAGTGGCACCG | | |
| | | AGTCGGTGCTTTTTT | | |
| | CXCR4 | GTTTTTAATACGACTCACTATAGAAGC | 7 | |
| | | GTGATGACAAAGAGGGTTTTAGAGCT | | |
| | | AGAAATAGCAAGTTAAAATAAGGCTA | | |
| | | GTCCGTTATCAACTTGAAAAAGTGGCA | | |
| | | CCGAGTCGGTGCTTTTTT | | |
| Primers for | GFP_FWD | CTGGTCGAGCTGGACGGCGACG | 8 | Amplicon |
| SURVEYOR | GFP_REV | CACGAACTCCAGCAGGACCATG | 9 | size: 630 bp |
| Assay | CXCR4_FWD | TTAATTCTCTTGTGCCCTTAGCCCACTA | 10 | Amplicon |
| | | CTTCAG | | size: 770 bp |
| | CXCR4_REV | GGACGGATGACAATACCAGGCAGGAT | 11 | |
| | | AAGGCC | | |
| HDR Donor | CXCR4 | CCTGGTCATGGGTTACCAGAAGAAACT | 12 | Inserted |
| Template | | GAGAAGCATGACGGACAAGTACAGGC | | Region |
| | | TGCACCTGTCAGTGGCCGACCTCCTAA | | Homology |
| | | GCTTGGATCCCTTTGTCATCACGCTTCC | | arms |
| | | CTTCTGGGCAGTTGATGCCGTGGCAAA | | HindIII |
| | | CTGGTACTTTGGGAACTTCCTATGCAA | | restriction |
| | | GGCAGTCCATGTCATCTA | | site |

### 1.7.7 Cell Culture and Cell Line Preparation

HEK-293T human embryonic kidney cells, GL261 murine glioma cells, CT-2A murine glioma cells, B16-F10 murine melanoma cells, and MSC-083 human primary adipose-derived mesenchymal stem cells (hAMSCs) were cultured in Dulbecco's Modified Eagle Medium (DMEM; ThermoFisher) supplemented with 10% FBS and 1% penicillin/streptomycin. To generate reporter cell lines for CRISPR editing experiments, HEK-293T and GL261 cells were induced to constitutively express a destabilized form of GFP (GFPd2) via a PiggyBac transposon/transposase system as detailed previously. Rui et al., 2019b. Similarly, GL261 and B16-F10 cells were induced to constitutively express a construct where transcription of a red-enhanced nanolantern (ReNL) reporter gene is prevented by a dual-SV40 transcription stop cassette (CRISPR-stop). Rui et al., 2019c. The PiggyBac transposon plasmids used to generate GFPd2+ and CRISPR-stop+ cell lines are available on Addgene as plasmids #115665 and #113965, respectively.

### 1.7.8 Transfection

Cells were plated at a density of 15,000 cells per well in 96-well tissue culture plates and allowed to adhere overnight. Protein-encapsulated nanoparticles were prepared as described above, and optimal nanoparticle formulations for each protein are listed in Table 1. 20 μL nanoparticles were added per well into serum-containing complete cell culture media and incubated with cells for 4 hours. For FITC-BSA uptake experiments, the nanoparticle/media mixture was removed at 4 hours, and cells were washed 3 times with PBS and uptake was assessed via flow cytometry using a BD Accuri C6 flow cytometer (BD Biosciences). Nanoparticle uptake was quantified by normalizing the geometric mean fluorescence of treated wells to that of untransfected controls.

For all other transfection experiments, fresh complete medium was replenished after 4 hours incubation with nanoparticles. For saporin transfection experiments, cell killing was assessed 2 days post-transfection. Cells were stained with Hoechst 33342 nuclear stain (1:5000 dilution) and propidium iodide (1:500 dilution) for 20 minutes and imaged and analyzed using Cellomics Arrayscan VTI with live cell imaging module (ThermoFisher). Cell killing was calculated by normalizing live cell numbers in wells treated with C₅/saporin nanoparticles to those in wells treated with matching nanoparticle formulations delivering non-toxic BSA. For CRISPR RNP transfection experiments, gene editing was assessed 3 days post-transfection. GFPd2 knockout and turning on of ReNL were assessed via flow cytometry. GFPd2 knockout was quantified by normalizing the GFP geometric mean fluorescence of C5/RNP treated wells to that of untransfected control wells; gain of ReNL fluorescence was quantified as the percentage of cells positively expressing ReNL when gated against untreated control.

For CRISPR HDR experiments, Cas9 and sgRNA targeting the CXCR4 gene were first mixed at a 1:2 molar ratio and allowed to self-assemble into RNPs. The ssDNA repair template was then added at a 1:1 volume ratio to the RNPs, and the combined solution was mixed with C5 polymer to allow for nanoparticle self-assembly. Each well received a final dose of 300 ng sgRNA, 690 ng Cas9 protein, and 400 ng ssDNA repair template.

### 1.7.9 Commercial Reagent RNP Delivery

For CRISPR RNP delivery experiments using commercial reagents, B16-F10 cells expressing the PiggyBac CRISPR-stop⁺ cassette were plated in 96-well plates 24 hours prior. Lipofectamine CRISPRMax and jetCRISPR commercial reagents designed for RNP delivery were formulated with SpCas9 RNP nanoparticles according to the manufacturer's instructions and added to cells at the specified doses. Specifically, RNPs were formulated as recommended at 1:1 molar ratio of SpCas9 to sgRNA using the single-guide CRISPR-stop sgRNA and complexed with commercial reagents before adding to cells and incubating 48 hours. Editing efficacy was assessed 2 days following RNP delivery using flow cytometry to assess percentage of cells expressing ReNL from the 630-bp deletion of the CRISPR-stop⁺ cassette.

### 1.7.10 Endocytosis Pathway Inhibition

HEK-293T cells were plated for transfection as described above and incubated for 1 hr with endocytosis inhibitors, dos Santos et al., 2011, in complete cell culture media immediately prior to transfection. Chlorpromazine (CPZ; 3 μg/mL) was used to inhibit clathrin-mediated endocytosis; methyl-β-cyclodextrin (MCD; 7.5 mg/mL) was used to inhibit lipid raft-mediated endocytosis; genistein (GEN; 10 μg/mL) was used to inhibit caveolin-mediated endocytosis; cytochalasin-D (CYD; 0.5 μg/mL) was used to inhibit actin polymerization and macropinocytosis. C₅/FITC-BSA nanoparticles were formulated at 300 ng protein per well and 30 w/w. Nanoparticles were incubated with cells for 2 hr, at which time they were washed with PBS and analyzed via flow cytometry to assess nanoparticle uptake. Endocytosis inhibition was quantified by normalizing the geometric mean fluorescence of wells treated with inhibitor to that of untransfected control wells.

### 1.7.11 Gal8-GFP Recruitment Assay

The Gal8-GFP recruitment assay to assess endosomal disruption was based on methods previously reported by Kilchrist et al., 2019. Briefly, B16-F10 cells were made to constitutively express a Gal8-GFP fusion protein using a PiggyBac transposon plasmid (Addgene 127191). Nanoparticles encapsulating BSA (125 ng BSA per well, 25 w/w) were incubated with cells for 4 hours, at which point cells were replenished with complete media and stained with Hoechst 33342 nuclear stain (1:5000 dilution). Gal8-GFP recruitment was imaged and analyzed with Cellomics Arrayscan VTI with live cell imaging module; cell count was generated using an algorithm to extrapolate area surrounding Hoechst-stained cell nuclei and endosomal disruption was reported as the average number of punctate Gal8-GFP spots per cell.

### 1.7.12 Cellular Viability

Cell viability was assessed 24 hours post-transfection using MTS CellTiter 96 Aqueous One cell proliferation assay (Promega) following manufacturer's instructions. Cell viability of treated cells were normalized to that of untreated controls; N=4+/−SEM.

### 1.7.13 Confocal Microscopy

HEK-293T or MSC-083 cells were plated on Nunc Lab-Tek 8 chambered borosilicate coverglass well plates (155411; ThermoFisher) at 30,000 cells/well in 250 μl phenol red free DMEM supplemented with 10% FBS and 1% penicillin/streptomycin one day prior to transfection. C₅ nanoparticles were prepared at 30 w/w with the indicated proteins and 504 nanoparticles were administered per well for a total dose of 300 ng protein. Nanoparticles were incubated with cells for 4 hours, at which time cells were replenished with fresh complete medium and stained with Hoechst 33342 nuclear stain at a 1:5000 dilution and Cell Navigator Lysosome Staining dye (AAT Bioquest). Excess stain was washed away and cells were imaged in live cell imaging solution at 37° C. in 5% CO₂.

Images were acquired at Nyquist limit resolution using a Zeiss LSM 780 microscope with Zen Blue software and 63× oil immersion lens. Specific laser channels used were 405-nm diode, 488-nm argon, 561-nm solid-state, and 639-nm diode lasers. Laser intensity and detector gain settings were maintained across all image acquisition for each experiment.

### 1.7.14 Indel Quantification Via Surveyor® Assay

Genomic DNA from cells treated with C5/RNP nanoparticles and untransfected controls were isolated using a GeneJET genomic DNA purification kit (ThermoFisher). A 660 bp region flanking the predicted cut site was PCR amplified (primers listed in Table 2), and PCR products were purified using a QIAquick PCR purification kit (Qiagen). 400 ng of PCR amplicons were hybridized in the presence of 50-mM KCl, and the Surveyor® mutation detection assay (IDT) was performed following manufacturer's instructions. The DNA products were then run on a 2% agarose gel stained with ethidium bromide in TBE buffer (80 V for 50 minutes) and imaged under UV light. DNA band intensity was quantified using ImageJ image analysis software and indel rate was calculated based on the method by Schumann et al., 2015.

### 1.7.15 HDR Quantification Via Restriction Enzyme Digest

A restriction enzyme-based assay to quantify HDR rates was adapted from methods reported by Lee et al., 2017. Briefly, a HDR repair template was designed to insert a 12-bp region that includes the HindIII restriction site into the CXCR4 gene, with 78 base homology arm upstream and 90 base homology arm downstream of the insert site. The repair template was synthesized as a single-stranded DNA oligo from IDT (sequence listed in Table 2). Genomic DNA of cells treated with C5/RNP+ssDNA nanoparticles or control nanoparticles was harvested 5 days post-transfection. A 770 bp region surrounding the edit site was PCR amplified, and the PCR amplicon was digested with HindIII (0.01 enzyme units/ng DNA) for 1 hr at 37° C. prior to standard gel electrophoresis as described above. Percent HDR was calculated by dividing the band intensity of the digested fragment (approximately 400 bp) by the band intensity of all bands in the lane.

### 1.7.16 GL261-CRISPR-Stop Tumor Implantation

All animal work was done in strict adherence of the policies and guidelines of the Johns Hopkins University Animal Care and Use Committee. For intracranial tumor implantations, 6-8 week old female C57BL/6J mice (Jackson Laboratory) were anesthetized using a 10 mg/kg ketamine (100 mg/kg)/Xylazine (10 mg/kg) cocktail and mounted on a stereotaxic frame. A rostro-caudal incision was made with a scalpel, the surface of the skull was exposed and cleaned with 100% ethanol, and a small burr hole was made in the skull 4 mm posterior to the coronal suture and 2 mm lateral to the sagittal suture using an electric drill. 130,000 cells (in 2 μL) were implanted into mouse brain parenchyma through the burr hole using a 10 μL Hamilton syringe (Hamilton Company).

### 1.7.17 Intratumoral C5/RNP Nanoparticle Injection

Tumors were allowed to form for 10 days, at which time C5/RNP nanoparticle administration began. Nanoparticles were formed in PBS buffer at a final polymer concentration of 0.86 mg/mL and 3.5 pmol RNPs (15 w/w) immediately prior to injection. Mice were anesthetized with a 10 mg/kg ketamine cocktail as described earlier, and the original incision was opened. Convection-enhanced delivery (CED) was performed using a 26 gauge Hamilton needle stereotaxically placed at a depth of 3 mm and an UltraMicroPump (UMP3) with SYS-Micro4 Controller (World Precision Instruments, Sarasota, Fla.) was used to infuse nanoparticles at a rate of 0.5 μL/min. Serwer et al., 2010. 10 μL of nanoparticles were injected per animal, after which the needle was left at the injection site for 5 min to mitigate backflow. Following needle removal, the incision was closed with 4-0 silk sutures and the animal was allowed to awaken and recover.

### 1.7.18 In Vivo Visualization of ReNL Reporter

For ReNL reporter analysis, 6-d post-injection mice were anesthetized and perfused with 4% paraformaldehyde. Brains were extracted, post-fixed overnight, and soaked in 30% sucrose for 24 h. Brains were frozen on dry ice and mounted onto a cryostat sample holder using Optimal Cutting Temperature compound (OCT), cryosectioned (coronal plane sections) using a Leica CM 3050 S cryostat (Leica Biosystem), and the prepared 40 μm sections were mounted onto glass slides with Hoechst nuclear stain (1:4000 dilution) and SlowFade® Gold Antifade Reagent (ThermoFisher). Mounted sections were stored at −80° C. and protected from light until use. Sections were imaged by fluorescence microscopy using a Zeiss Apotome.2 microscope with Zen Blue software. Microscope settings were maintained across all image acquisition.

### 1.7.19 Nanoparticle Stability

To characterize nanoparticle stability over time in physiological conditions, C5/RNP nanoparticles were incubated in serum-containing complete cell culture medium at 37° C. and added to GL261-CRISPR-stop cells at designated time points up to 4 h. C5/RNP nanoparticles also were lyophilized with 30 mg/mL sucrose as cryoprotectant following previously-reported protocols, Lopez-Bertoni et al., 2018, and stored at −20° C. for 4 days before adding to cells. Cells were incubated with nanoparticles for 3 h and the level of gene editing was analyzed via flow cytometry 3-days post-transfection.

### 1.7.20 Statistics

Prism 6 (Graphpad, La Jolla, Calif.) was used for all statistical analyses and curve plotting. Statistical tests were performed with a global alpha value of 0.05. Unless otherwise stated, absence of statistical significance markings where a test was stated to have been performed signified no statistical significance. The statistical test used and the number of experimental replicates were listed in the captions for each figure. Statistical significance was denoted as follows: *P<0.05, **P<0.01, ***P<0.001, ****P<0.0001.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Clark, A. L.; Knight, G.; Wiles, P.; Keen, H.; Ward, J.; Cauldwell, J.; Adeniyi-Jones, R.; Leiper, J.; Jones, R.; Maccuish, A., Biosynthetic human insulin in the treatment of diabetes: a double-blind crossover trial in established diabetic patients. The Lancet 1982, 320 (8294), 354-357.
Leader, B.; Baca, Q. J.; Golan, D. E., Protein therapeutics: a summary and pharmacological classification. Nature Reviews Drug Discovery 2008, 7, 21.
Paschon, D. E.; Lussier, S.; Wangzor, T.; Xia, D. F.; Li, P. W.; Hinkley, S. J.; Scarlott, N. A.; Lam, S. C.; Waite, A. J.; Truong, L. N.; Gandhi, N.; Kadam, B. N.; Patil, D. P.; Shivak, D. A.; Lee, G. K.; Holmes, M. C.; Zhang, L.; Miller, J. C.; Rebar, E. J., Diversifying the structure of zinc finger nucleases for high-precision genome editing. Nature Communications 2019, 10 (1), 1133.
Lienert, F.; Lohmueller, J. J.; Garg, A.; Silver, P. A., Synthetic biology in mammalian cells: next generation research tools and therapeutics. Nature reviews Molecular cell biology 2014, 15 (2), 95.
Postupalenko, V.; Desplancq, D.; Orlov, I.; Arntz, Y.; Spehner, D.; Mely, Y.; Klaholz, B. P.; Schultz, P.; Weiss, E.; Zuber, G., Protein Delivery System Containing a Nickel-Immobilized Polymer for Multimerization of Affinity-Purified His-Tagged Proteins Enhances Cytosolic Transfer. Angewandte Chemie International Edition 2015, 54 (36), 10583-10586.
Southwell, A. L.; Khoshnan, A.; Dunn, D. E.; Bugg, C. W.; Lo, D. C.; Patterson, P. H., Intrabodies binding the proline-rich domains of mutant huntingtin increase its turnover and reduce neurotoxicity. The Journal of neuroscience: the official journal of the Society for Neuroscience 2008, 28 (36), 9013-9020.
Rui, Y.; Wilson, D. R.; Green, J. J., Non-Viral Delivery To Enable Genome Editing. Trends in Biotechnology 2019a, 37 (3), 281-293.
Schwarze, S. R.; Ho, A.; Vocero-Akbani, A.; Dowdy, S. F., In Vivo Protein Transduction: Delivery of a Biologically Active Protein into the Mouse. Science 1999, 285 (5433), 1569.
Wang, M.; Zuris, J. A.; Meng, F.; Rees, H.; Sun, S.; Deng, P.; Han, Y.; Gao, X.; Pouli, D.; Wu, Q.; Georgakoudi, I.; Liu, D. R.; Xu, Q., Efficient delivery of genome-editing proteins using bioreducible lipid nanoparticles. Proceedings of the National Academy of Sciences 2016, 113 (11), 2868.
Zhang, Z.; Shen, W.; Ling, J.; Yan, Y.; Hu, J.; Cheng, Y., The fluorination effect of fluoroamphiphiles in cytosolic protein delivery. Nature Communications 2018, 9 (1), 1377.
Chang, H.; Lv, J.; Gao, X.; Wang, X.; Wang, H.; Chen, H.; He, X.; Li, L.; Cheng, Y., Rational design of a polymer with robust efficacy for intracellular protein and peptide delivery. Nano letters 2017, 17 (3), 1678-1684.
Cheng, G.; Li, W.; Ha, L.; Han, X.; Hao, S.; Wan, Y.; Wang, Z.; Dong, F.; Zou, X.; Mao, Y., Self-Assembly of Extracellular Vesicle-like Metal—Organic Framework Nanoparticles for Protection and Intracellular Delivery of Biofunctional Proteins. Journal of the American Chemical Society 2018.
Chen, T.-T.; Yi, J.-T.; Zhao, Y.-Y.; Chu, X., Biomineralized Metal-Organic Framework Nanoparticles Enable Intracellular Delivery and Endo-Lysosomal Release of Native Active Proteins. Journal of the American Chemical Society 2018.
Alsaiari, S. K.; Patil, S.; Alyami, M.; Alamoudi, K. O.; Aleisa, F. A.; Merzaban, J. S.; Li, M.; Khashab, N. M., Endosomal Escape and Delivery of CRISPR/Cas9 Genome Editing Machinery Enabled by Nanoscale Zeolitic Imidazolate Framework. Journal of the American Chemical Society 2017, 140 (1), 143-146.
Gao, Y.; Huang, J.-Y.; O'Keeffe Ahern, J.; Cutlar, L.; Zhou, D.; Lin, F.-H.; Wang, W., Highly Branched Poly(β-amino esters) for Non-Viral Gene Delivery: High Transfection Efficiency and Low Toxicity Achieved by Increasing Molecular Weight. Biomacromolecules 2016, 17 (11), 3640-3647.
Wilson, D. R.; Rui, Y.; Siddiq, K.; Routkevitch, D.; Green, J. J., Differentially Branched Ester Amine Quadpolymers with Amphiphilic and pH-Sensitive Properties for Efficient Plasmid DNA Delivery. Mol Pharm 2019, 16 (2), 655-668.
Rui, Y.; Wilson, D. R.; Sanders, K.; Green, J. J., Reducible Branched Ester-Amine Quadpolymers (rBEAQs) Codelivering Plasmid DNA and RNA Oligonucleotides Enable CRISPR/Cas9 Genome Editing. ACS Applied Materials & Interfaces 2019b, 11 (11), 10472-10480.
Shmueli, R. B.; Sunshine, J. C.; Xu, Z.; Duh, E. J.; Green, J. J., Gene delivery nanoparticles specific for human microvasculature and macrovasculature. Nanomedicine: Nanotechnology, Biology and Medicine 2012, 8 (7), 1200-1207.
Kilchrist, K. V.; Dimobi, S. C.; Jackson, M. A.; Evans, B. C.; Werfel, T. A.; Dailing, E. A.; Bedingfield, S. K.; Kelly, I. B.; Duvall, C. L., Gal8 Visualization of Endosome Disruption Predicts Carrier-Mediated Biologic Drug Intracellular Bioavailability. ACS Nano 2019, 13 (2), 1136-1152.
Wilson, D. R.; Mosenia, A; Suprenant, M. P.; Upadhya, R; Routkevitch, D; Meyer, R. A.; Quinones-Hinojosa, A; Green, J. J., Continuous microfluidic assembly of biodegradable poly(beta-amino ester)/DNA nanoparticles for enhanced gene delivery. J. Biomed. Mater. Res. A., 2017, 105, 1813-1835.
Lombardi, A.; Marshall, R. S.; Savino, C.; Fabbrini, M. S.; Ceriotti, A., Type I Ribosome-Inactivating Proteins from Saponaria officinalis. In Toxic Plant Proteins, Lord, J. M.; Hartley, M. R., Eds. Springer Berlin Heidelberg: Berlin, Heidelberg, 2010; pp 55-78.
Hsiau, T.; Maures, T.; Waite, K.; Yang, J.; Kelso, R.; Holden, K.; Stoner, R., Inference of crispr edits from sanger trace data. BioRxiv 2018, 251082.
Sunshine, J. C.; Peng, D. Y.; Green, J. J., Uptake and transfection with polymeric nanoparticles are dependent on polymer end-group structure, but largely independent of nanoparticle physical and chemical properties. Mol Pharm 2012, 9 (11), 3375-3383.
Guerrero-Cázares, H.; Tzeng, S. Y.; Young, N. P.; Abutaleb, A. O.; Quiñones-Hinojosa, A.; Green, J. J., Biodegradable polymeric nanoparticles show high efficacy and specificity at DNA delivery to human glioblastoma in vitro and in vivo. ACS Nano 2014, 8 (5), 5141-5153.
Rehman, Z. u.; Hoekstra, D.; Zuhorn, I. S., Mechanism of Polyplex- and Lipoplex-Mediated Delivery of Nucleic Acids: Real-Time Visualization of Transient Membrane Destabilization without Endosomal Lysis. ACS Nano 2013, 7 (5), 3767-3777.
Ayala, R.; Zhang, C.; Yang, D.; Hwang, Y.; Aung, A.; Shroff, S. S.; Arce, F. T.; Lal, R.; Arya, G.; Varghese, S., Engineering the cell—material interface for controlling stem cell adhesion, migration, and differentiation. Biomaterials 2011, 32 (15), 3700-3711.
Rui, Y.; Quiñones, G.; Green, J. J., Biodegradable and bioreducible poly(beta-amino ester) nanoparticles for intracellular delivery to treat brain cancer. AIChE Journal 2017, 63 (5), 1470-1482.
Sun, W.; Ji, W.; Hall, J. M.; Hu, Q.; Wang, C.; Beisel, C. L.; Gu, Z., Self-Assembled DNA Nanoclews for the Efficient Delivery of CRISPR—Cas9 for Genome Editing. Angewandte Chemie International Edition 2015, 54 (41), 12029-12033.
Lee, K.; Conboy, M.; Park, H. M.; Jiang, F.; Kim, H. J.; Dewitt, M. A.; Mackley, V. A.; Chang, K.; Rao, A.; Skinner, C.; Shobha, T.; Mehdipour, M.; Liu, H.; Huang, W.-C.; Lan, F.; Bray, N. L.; Li, S.; Corn, J. E.; Kataoka, K.; Doudna, J. A.; Conboy, I.; Murthy, N., Nanoparticle delivery of Cas9 ribonucleoprotein and donor DNA in vivo induces homology-directed DNA repair. Nature biomedical engineering 2017, 1, 889-901.
Staahl, B. T.; Benekareddy, M.; Coulon-Bainier, C.; Banfal, A. A.; Floor, S. N.; Sabo, J. K.; Umes, C.; Munares, G. A.; Ghosh, A.; Doudna, J. A., Efficient genome editing in the mouse brain by local delivery of engineered Cas9 ribonucleoprotein complexes. Nature Biotechnology 2017, 35, 431.
Rui, Y. V., Mahita; Mendes, Shanelle; Wilson, David R.; Green, Jordan J., Poly(beta-amino ester) nanoparticles enable non-viral delivery of CRISPR/Cas9 plasmids for gene knockout and gene deletion. Molecular Therapy Nucleic Acids 2019c, In Review.
Patel, A. K.; Kaczmarek, J. C.; Bose, S.; Kauffman, K. J.; Mir, F.; Heartlein, M. W.; DeRosa, F.; Langer, R.; Anderson, D. G., Inhaled nanoformulated mRNA polyplexes for protein production in lung epithelium. Adv. Mater. 2019, 31, 1805116.
Charlesworth, C. T.; Deshpande, P. S.; Dever, D. P.; Camarena, J.; Lemgart, V. T.; Cromer, M. K.; Vakulskas, C. A.; Collingwood, M. A.; Zhang, L.; Bode, N. M.; Behlke, M. A.; Dejene, B.; Cieniewicz, B.; Romano, R.; Lesch, B. J.; Gomez-Ospina, N.; Mantri, S.; Pavel-Dinu, M.; Weinberg, K. I.; Porteus, M. H., Identification of preexisting adaptive immunity to Cas9 proteins in humans. Nat. Med. 2019, 25, 249-254.
dos Santos, T.; Varela, J.; Lynch, I.; Salvati, A.; Dawson, K. A., Effects of Transport Inhibitors on the Cellular Uptake of Carboxylated Polystyrene Nanoparticles in Different Cell Lines. PLOS ONE 2011, 6 (9), e24438.
Schumann, K.; Lin, S.; Boyer, E.; Simeonov, D. R.; Subramaniam, M.; Gate, R. E.; Haliburton, G. E.; Ye, C. J.; Bluestone, J. A.; Doudna, J. A.; Marson, A., Generation of knock-in primary human T cells using Cas9 ribonucleoproteins. Proceedings of the National Academy of Sciences 2015, 112 (33), 10437.
Serwer, L.; Hashizume, R.; Ozawa, T.; James, C. D., Systemic and local drug delivery for treating diseases of the central nedefrvous system in rodent models. Journal of visualized experiments: JoVE 2010, (42), 1992.
Lopez-Bertoni, H.; Kozielski, K. L.; Rui, Y.; Lal, B.; Vaughan, H.; Wilson, D. R.; Mihelson, N.; Eberhart, C. G.; Laterra, J.; Green, J. J., Bioreducible Polymeric Nanoparticles Containing Multiplexed Cancer Stem Cell Regulating miRNAs Inhibit Glioblastoma Growth and Prolong Survival. Nano Lett 2018, 18 (7), 4086-4094.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a cationic core polymer, which preferably is biodegradable, having one or more anionic end groups having a functional group selected from a carboxylate, a phosphate, and a sulfonate, and a biomolecule, preferably an amino-acid containing biomolecule.

2. The composition of claim 1, wherein the cationic polymer comprises a naturally-derived cationic polymer.

3. The composition of claim 2, wherein the naturally-derived cationic polymer is selected from the group consisting of chitosan, gelatin, dextran, cellulose, cyclodextrin, and a polypeptide, and/or other naturally-derived cationic polymers.

4. The composition of claim 1, wherein the cationic polymer comprises a synthetic cationic polymer.

5. The composition of claim 4, wherein the synthetic cationic polymer is selected from the group consisting of a polyethyleneimine (PEI), poly-L-lysine (PLL), a poly(amidoamine) (PAA), a poly(amino-co-ester) (PAE), poly(2-N,N-dimethylaminoethylmethacrylate, a poly(beta-amino ester) (PBAE), an imidazole-containing polymer, a tertiary-amine containing polymer, poly(2-(dimethylamino)ethyl methacrylate), poly-N-(2-hydroxy-propyl)methacrylamide, polyamidoamine dendrimers, or derivatives thereof.

6. The composition of claim 1, wherein the one or more anionic end groups is selected from the group consisting of an amide-linked carboxylate, an ester-linked carboxylate, an ester-ethylene glycol-linked carboxylate, an amide-ethylene glycol-linked carboxylate, an ester-linked phosphate, an ester-ethylene glycol-linked phosphate, an amide-ethylene glycol-linked phosphate, an ester-linked sulfonate, and an ester-ethylene glycol-linked sulfonate, an amide-ethylene glycol-linked sulfonate.

7. The composition of claim 1, wherein the one or more anionic end groups is selected from the group consisting of: 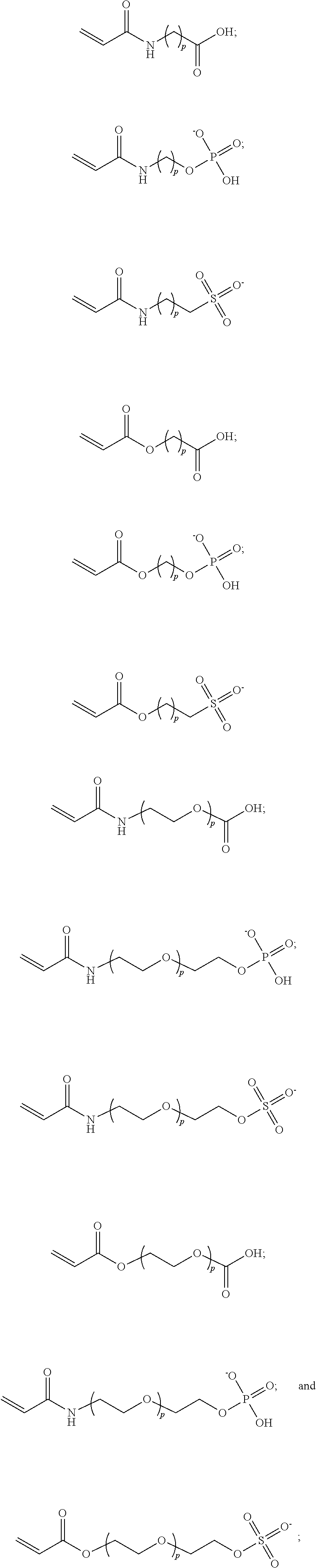
wherein p is an integer from 1, 2, 3, 4, 5, 6, 7, 8, 9, and 10.

8. The composition of claim 1, wherein the composition comprises a branched poly(beta-amino ester) (PBAE) of formula (I) or formula (II): 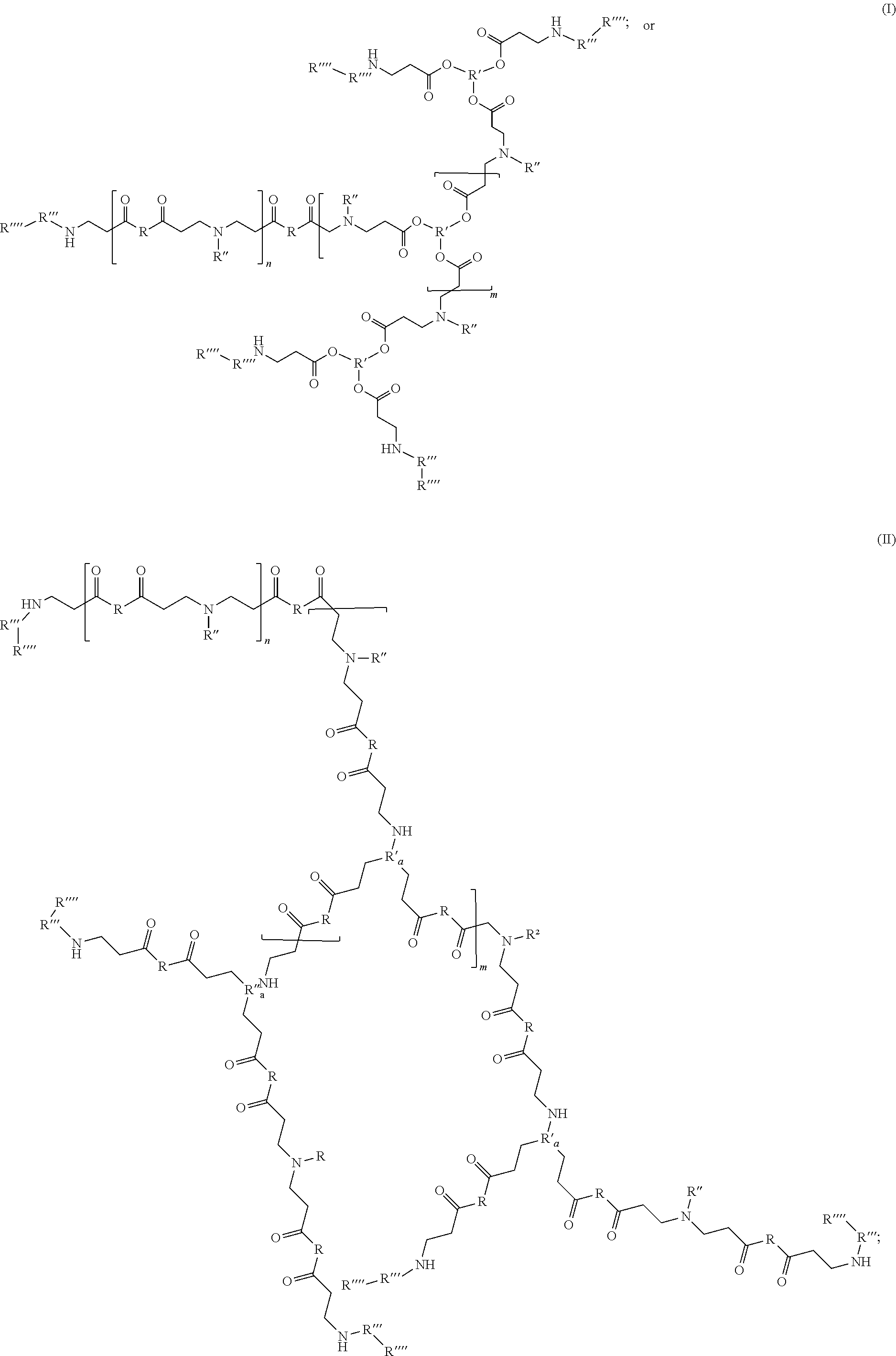
wherein:
n and m are each independently an integer from 1 to 10,000;
each R is independently a diacrylate monomer of the following structure: 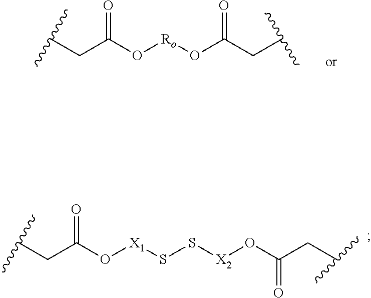
wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain;
each R′ of a compound of formula (I) is a trivalent group of a triacrylate monomer having the following structure: 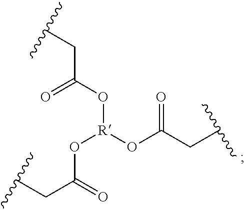
each R′ₐ of a compound of formula (II) is a tri-functional amine;
each R″ is independently a side chain monomer comprising a primary, secondary, or tertiary amine;
each R′″ is independently an end group monomer comprising a primary, secondary, or tertiary amine;
each R″″ is independently an anionic end group; and
pharmaceutically acceptable salts thereof.

9. The composition of claim 8, wherein R is selected from the group consisting of:

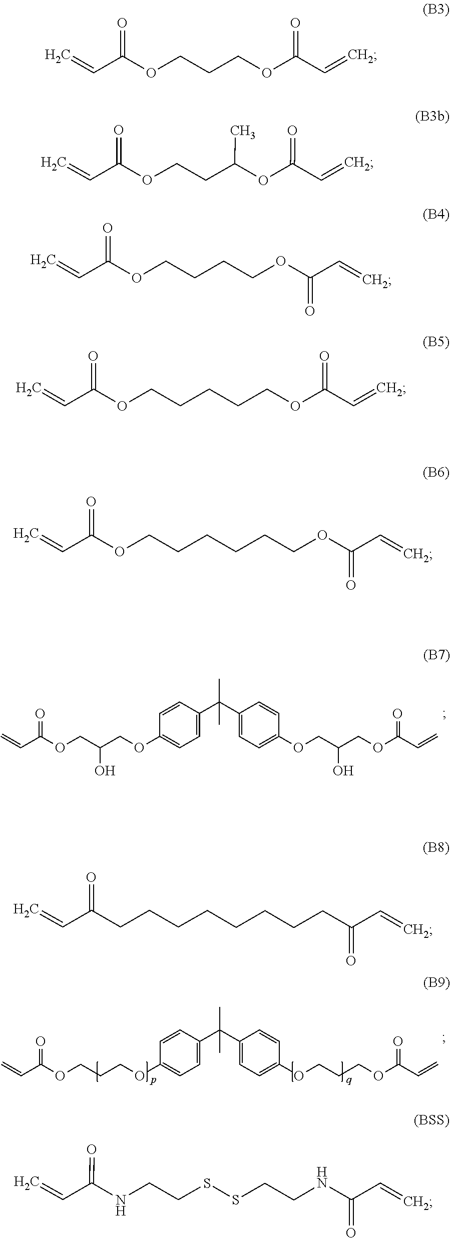

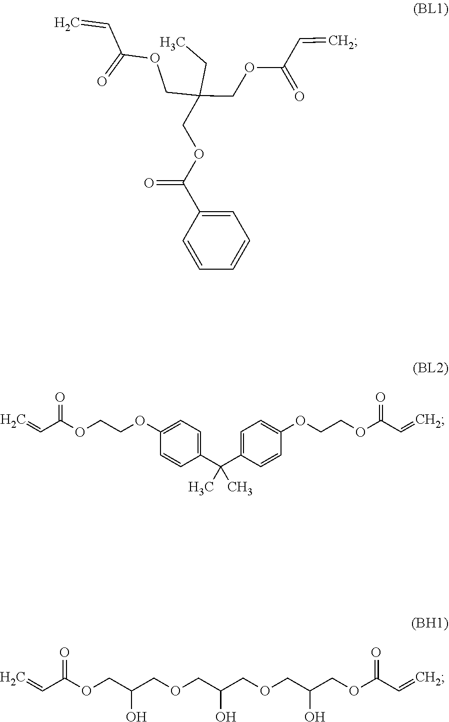 [SMILES: C=CC(=O)OCC(CC)(COC(=O)C=C)COC(=O)c1ccccc1 ; C=CC(=O)OCCOc1ccc(C(C)(C)c2ccc(OCCOC(=O)C=C)cc2)cc1 ; C=CC(=O)OCC(O)COCC(O)COCC(O)COC(=O)C=C]

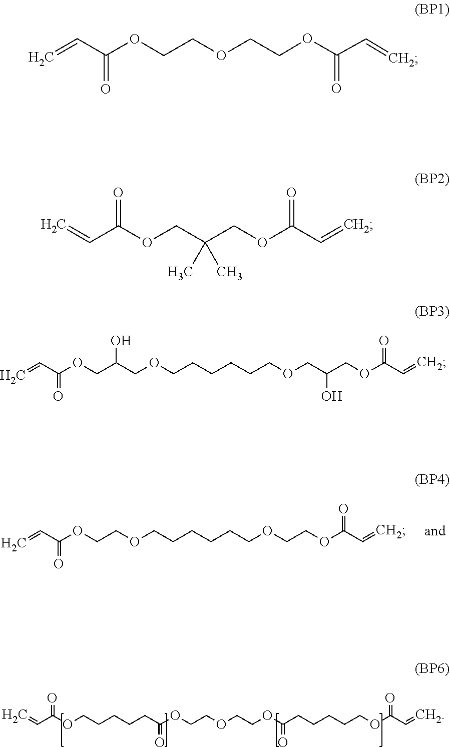

10. The composition of claim 8, wherein the trivalent group R′ is —C—CH₂CH₃ and the triacrylate monomer is trimethylolpropane triacrylate (TMPTA):

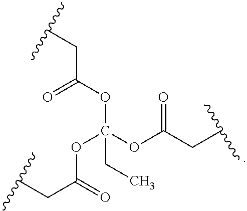 [SMILES: *CC(=O)OC(CC)(OC(=O)C*)OC(=O)C*]

11. The composition of claim 8, wherein the tri-functional amine monomer of formula (II) is selected from the group consisting of:

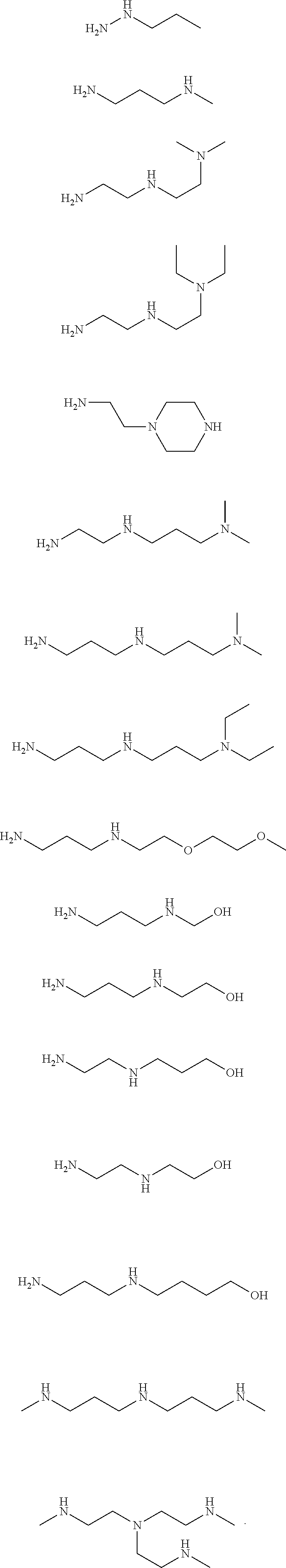

12. The composition of claim 8, wherein R″ is selected from the group consisting of:

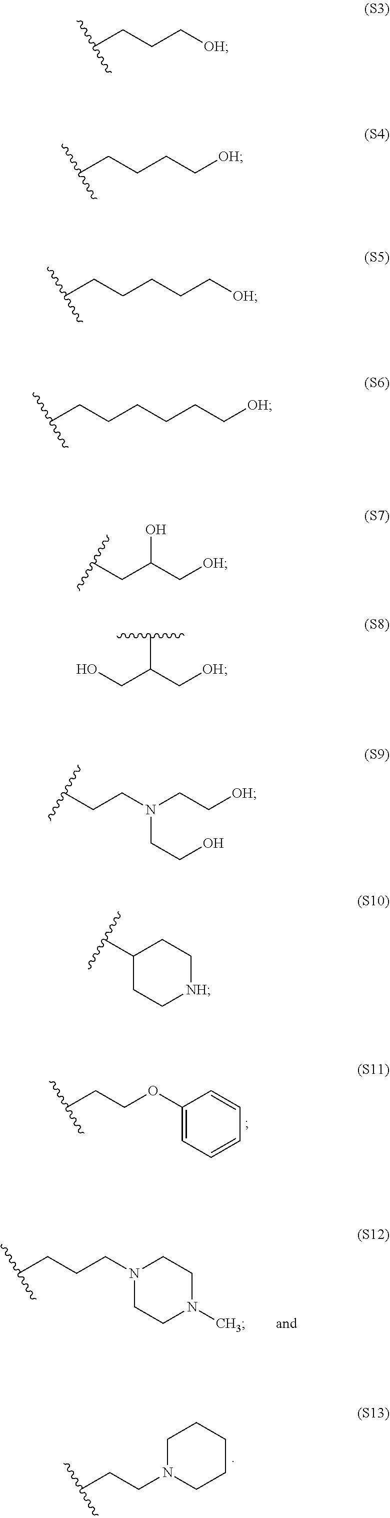

13. The composition of claim 8, wherein R″ is selected from the group consisting of:

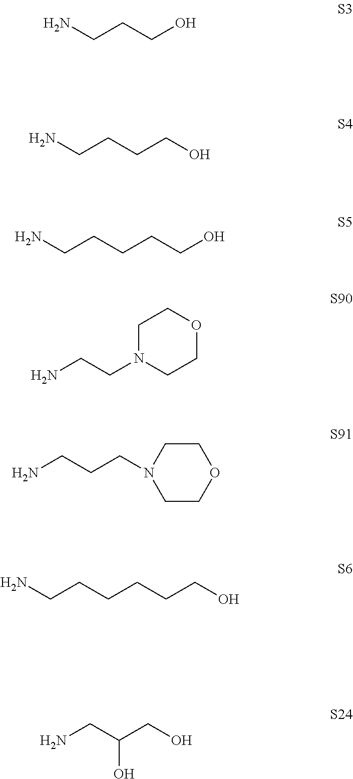

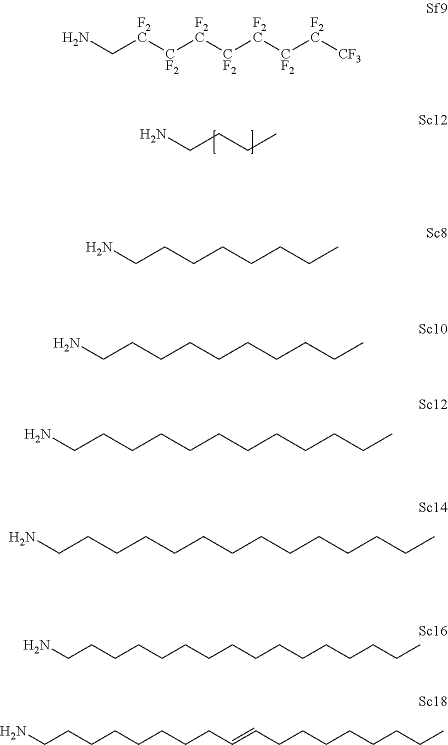

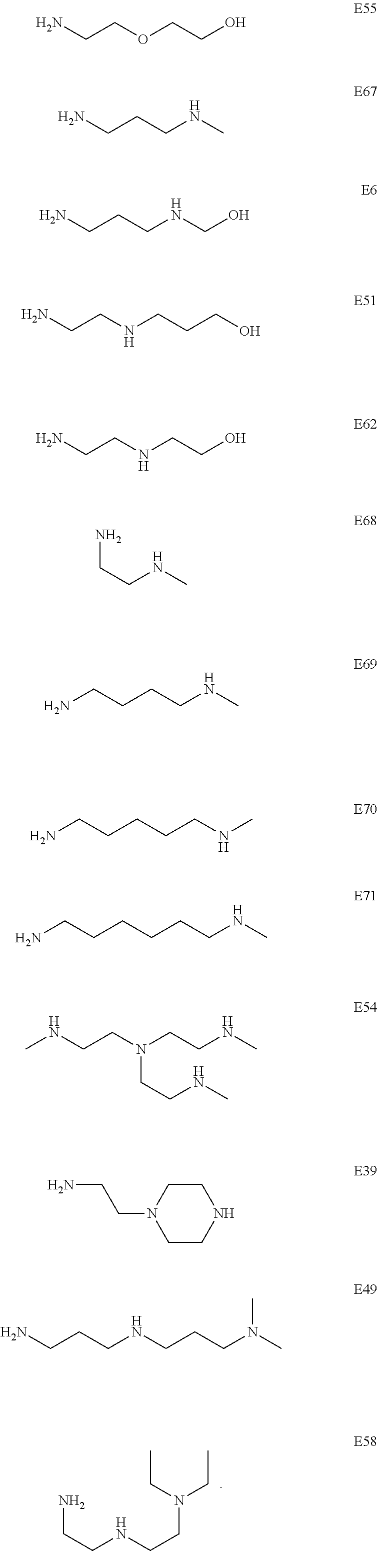

14. The composition of claim 8, wherein R′″ is selected from the group consisting of:
Amino Alkanes

| | |
|---|---|
| 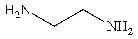 | A1 |
| 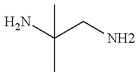 | A2 |
| 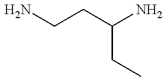 | A3 |
| 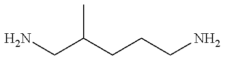 | A4 |
| 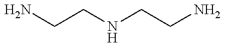 | A5 |
| 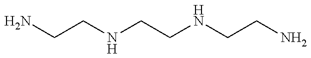 | A6 |
| 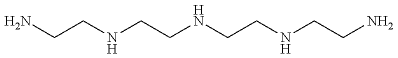 [SMILES: NCCNCCNCCNCCN] | A7 |
| 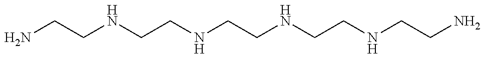 [SMILES: NCCNCCNCCNCCNCCN] | A8 |
| 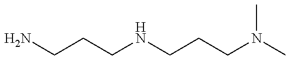 | A9 |
| 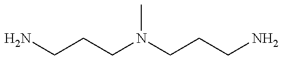 | A10 |
| 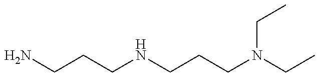 | A11 |
| 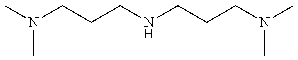 | A12 |
| 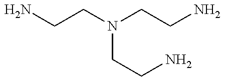 | A13 |
| 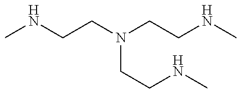 | A14 |

Amino Piperidines

| | |
|---|---|
| 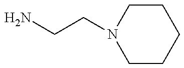 | B1 |
| 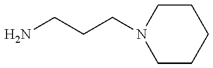 | B2 |
| 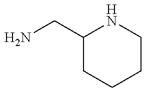 | B3 |
| 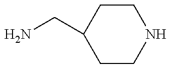 | B4 |

Amino Pyrrolidines

| | |
|---|---|
| 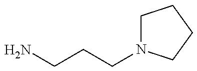 | D1 |
| 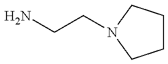 | D2 |

Amino Alcohols

| | |
|---|---|
| 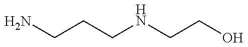 | E1 |
| 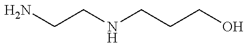 | E2 |
| 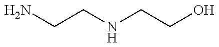 | E3 |
| 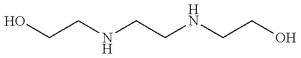 | E4 |
| 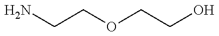 | E5 |
| 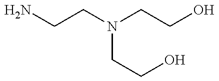 | E6 |

Amino Piperizines

| | |
|---|---|
| 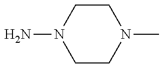 | C1 |
| 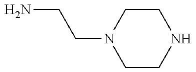 | C2 |
| 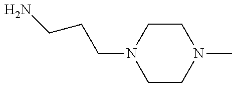 | C3 |
| 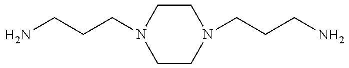 [SMILES: NCCCN1CCN(CCCN)CC1] | C4 |

Diamino ethers

| | |
|---|---|
| 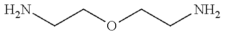 | F1 |
| 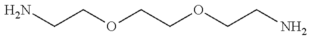 | F2 |
| 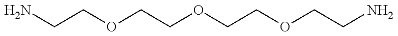 [SMILES: NCCOCCOCCOCCN] | F3 |
| 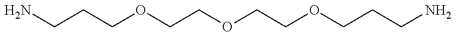 [SMILES: NCCCOCCOCCOCCCN] | F4 |

Amino morpholinos

| | |
|---|---|
| 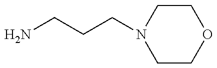 | G1 |
| 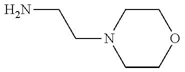 | G2 |

15. The composition of claim 8, wherein R′″ is selected from the group consisting of:

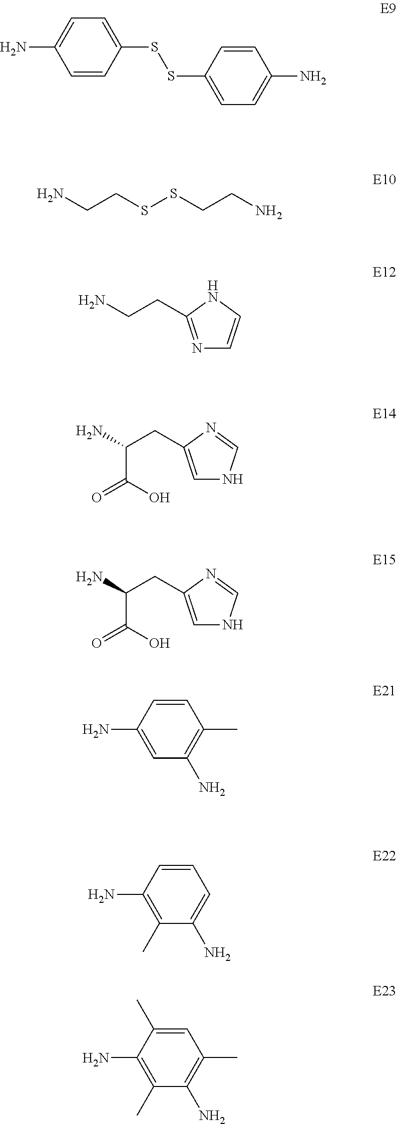

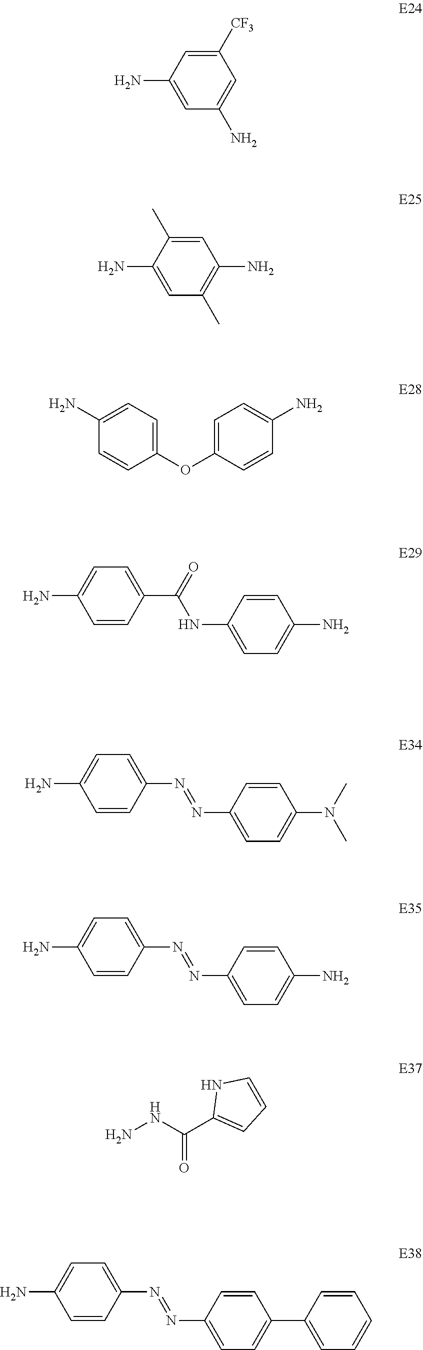

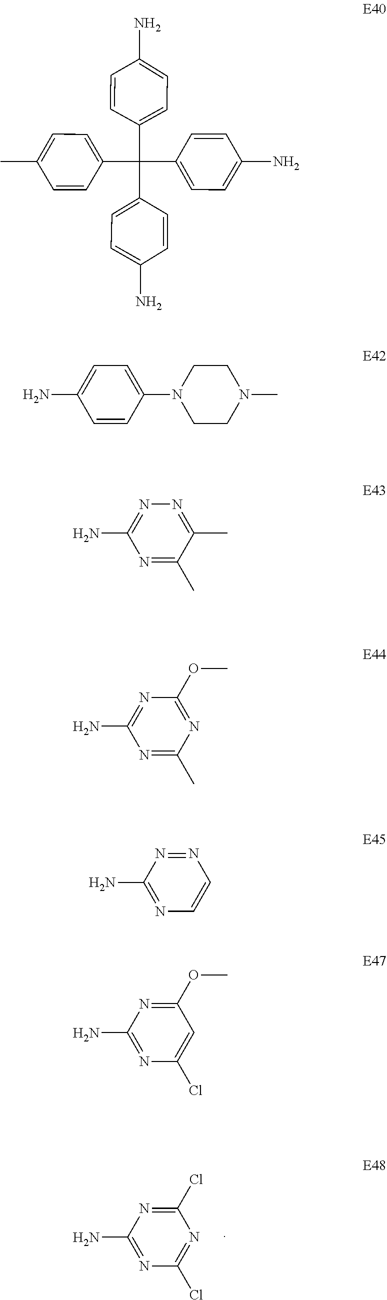

16. The composition of claim 8, wherein R″″ is selected from the group consisting of: 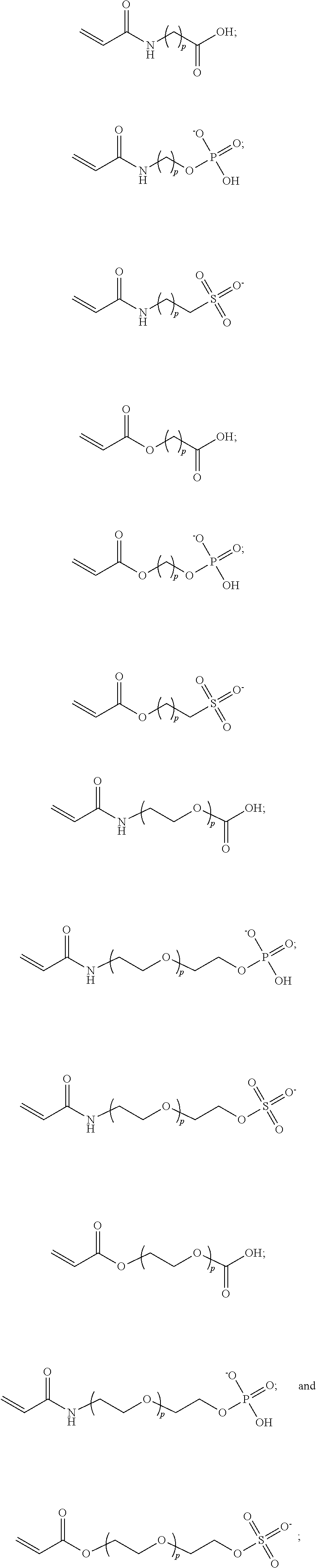
wherein p is an integer from 1, 2, 3, 4, 5, 6, 7, 8, 9, and 10.

17. The composition of claim 16, wherein R″″ is: 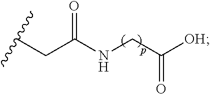
wherein p is an integer from 1, 2, 3, 4, 5, 6, 7, 8, 9, and 10.

18. The composition of claim 17, wherein R″″ is selected from the group consisting of:

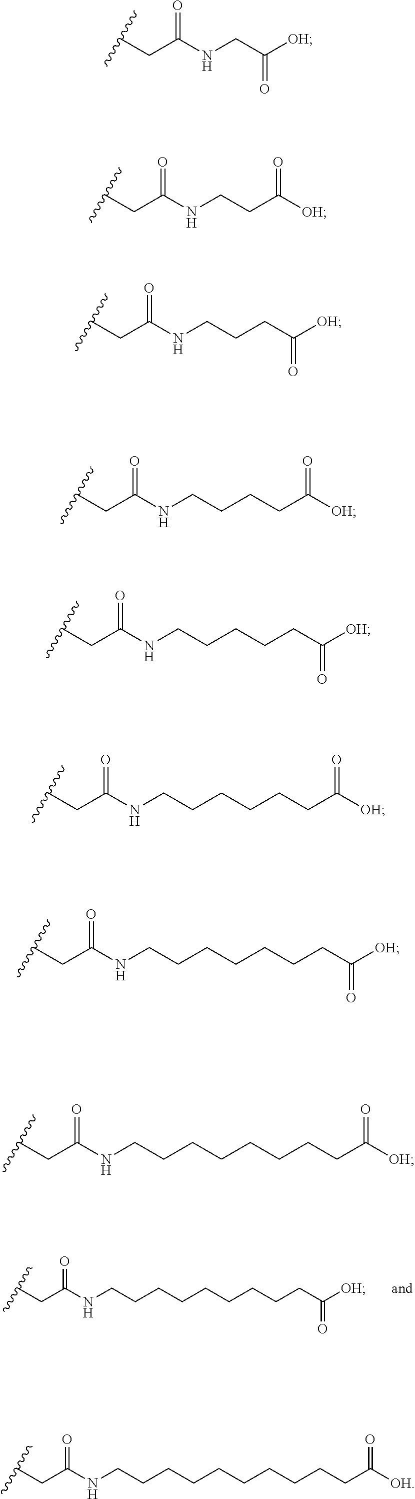

19. The composition of claim 8, wherein n and m are each independently selected from the group consisting of: an integer from 1 to 1,000; an integer from 1 to 100; an integer from 1 to 30; an integer from 5 to 20; an integer from 10 to 15; and an integer from 1 to 10.

20. The composition of claim 1, further comprising one or more biomolecules selected from a peptide, a protein, a nucleic acid, a morpholino, and other charged or zwitterionic biomolecules or combinations thereof.

21. The composition of claim 20, wherein the one or more biological molecules is selected from the group consisting of a non-peptide based biological small molecule or a biomacromolecule selected from a sugar, a polysaccharide, a carbohydrate, a morpholino oligomer, and/or a nucleic acid.

22. The composition of claim 21, wherein the one or more biomolecules comprises a protein.

23. The composition of claim 22, wherein the protein is selected from the group consisting of a ribosome inactivating protein (RIP), a gene-editing protein, an immunoglobulin, a nanobody, and an intrabody.

24. The composition of claim 23, wherein the ribosome inactivating protein (RIP) is selected from the group consisting of abrin, beetin, ricin, saporin, Shiga toxin, a Spiroplasma protein, trichosanthin, and viscumin.

25. The composition of claim 22, wherein the protein comprises a gene editing protein.

26. The composition of claim 25, wherein the gene editing protein comprises a Cas9 ribonucleoprotein (RNP).

27. The composition of claim 26, further comprising a guide RNA (gRNA).

28. The composition of claim 22, wherein the protein is labeled with one or more ligands suitable for detecting the protein in a cell.

29. The composition of claim 28, wherein the label comprises a fluorescent label.

30. The composition of claim 29, wherein the fluorescent label is selected from the group consisting of fluorescein isothiocyanate (FITC), green fluorescent protein (GFP), AlexaFluor 350, AlexaFluor 430, AlexaFluor405, AlexaFluor488, AlexaFluor546, AlexaFluor555, AlexaFluor594, AlexaFluor660, AlexaFluor633, AlexaFluor647, AlexaFluor680, AlexaFluor700, AlexaFluor750, AlexaFluor790, AMCA, (BODIPY) dye, or derivatives thereof, including, but not limited to, BODIPY 630/650, BODIPY 650/665, BODIPY 581/591, BODIPY-FL, BODIPY-R6G, BODIPY-TR, BODIPY-TMR, BODIPY-TRX, Cascade Blue, Cy3, Cy5, Cy5.5, Cy7, 6-FAM, fluorescein, Fluorescein Isothiocyanate, TRITC, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, Texas Red, carbocyanine, indocarbocyanine, oxacarbocyanine, thuicarbocyanine, merocyanine, polymethine, coumarine, rhodamine, xanthene, a boron-dipyrromethane VivoTag-680, VivoTag-S680, VivoTag-S750, Dy677, Dy676, Dy682, Dy752, Dy780, DyLight547, DyLight647, DyLight 350 (Ex/Em=353 nm/432 nm), DyLight 405 (400/420), DyLight 488 (493/518), DyLight 550 (562/576), DyLight 594 (593/618), DyLight 633 (638/658), DyLight 650 (652/672), DyLight 680 (692/712), DyLight 755 (754/776), DyLight 800 (777/794), and derivatives thereof, including, but not limited to, NHS esters, maleimides, phosphines, and free acids, HiLyte Fluor 647, HiLyte Fluor 680, HiLyte Fluor 750, IR800 (EHmethyl{4-[1,5,5-tris(4-dimethylaminophenyl)-2,4-pentadienylidene]-2,5-cyclohexadien-1-ylidene} ammonium perchlorate), IRDye 800CW, IRDye 800RS, IRDye 700DX, ADS780WS, ADS830WS, ADS832WS, R-Phycoerythrin, Flamma749, Flamma774, and ICG.

31. A pharmaceutical formulation comprising the composition of any of claims 1-30 in a pharmaceutically acceptable carrier.

32. The pharmaceutical formulation of claim 31, further comprising a nanoparticle or microparticle of the cationic polymer having one or more anionic end groups.

33. The pharmaceutical formulation of claim 32, wherein the nanoparticle or microparticle is encapsulated in a poly(lactic-co-glycolic acid) (PLGA) nanoparticle or microparticle.

34. The pharmaceutical formulation of claim 32, wherein the nanoparticle is lyophilized.

35. The pharmaceutical formulation of claim 34, wherein the nanoparticle is lyophilized with a cryoprotectant.

36. The pharmaceutical formulation of claim 35, wherein the cryoprotectant comprises a sugar.

37. A kit comprising the composition of any of claims 1-30 or the pharmaceutical formulation of any of claims 31 to 36.

38. The kit of claim 37, further comprising one of more of multiple dosage units of the composition, a pharmaceutically acceptable carrier, a device for administration of the composition, instructions for use, and combinations thereof.

39. A method for delivering a protein to a cell, the method contacting a cell with the composition of any one of claims 1 to 30 or the formulation of any of claims 31 to 36, wherein the composition comprises at least one protein.

40. The method of claim 39, wherein the protein is delivered to a cytosol of the cell.

41. The method of claim 39, wherein the method mediates endosomal disruption.

42. A method for editing a gene, comprising contacting a cell with the composition of any one of claims 1 to 39 or the formulation of any of claims 31 to 36, wherein the composition or formulation comprises at least one gene-editing protein.

43. The method of claim 42, wherein the gene-editing protein comprises a Cas9 ribonucleoprotein (RNP).

44. The method of claim 43, wherein the Cas9 ribonucleoprotein (RNP) directs site-specific target DNA disruption, mutation, deletion, or repair.

45. The method of claim 42, wherein the composition and cell are contacted in vivo.

46. The method of claim 42, wherein the composition and cell are contacted ex vivo.

47. The method of claim 42, wherein the cell is a eukaryotic cell.

48. The method of claim 47, wherein the cell is an animal cell or plant cell.

49. The method of claim 48, wherein the animal cell is a mammalian cell.

50. The method of claim 42, wherein the cell is a human cell.

51. The method of claim 42, wherein the cell is a stem cell or progenitor cell.

52. The method of claim 51, wherein the cell is multipotent or pluripotent.
